(19) United States
(12) Patent Application Publication
(10) Pub. No.: US 2005/0266242 A1
(43) Pub. Date: Dec. 1, 2005

(54) Electrical conductors and devices from prion-like proteins

(76) Inventors: Susan Lindquist, Chestnut Hill, MA (US); Thomas Scheibel, Muenchen (DE)

Correspondence Address: MARSHALL, GERSTEIN & BORUN LLP, 233 S. WACKER DRIVE, SUITE 6300, SEARS TOWER, CHICAGO, IL, 60606, US

(21) Appl. No.: 11/089,551

(22) Filed: Mar. 24, 2005

Related U.S. Application Data
(60) Provisional application No. 60/559,286, filed on Mar. 31, 2004.

(51) Int. Cl. D02G 3/00

(52) U.S. Cl. 428/375; 427/375

(57) ABSTRACT

The present invention provides novel polypeptides comprising a prion-aggregation domain and a second domain; novel polynucleotides encoding such polypeptides; host cells transformed or transfected with such polynucleotides; novel fibrils with specific functionalities and unusually high chemical and thermal stability; and methods of making and using the foregoing.

This application claims priority benefit of U.S. Provisional Patent Application No. 60/559,286, filed Mar. 31, 2004. All priority applications are hereby incorporated by reference in their entirety.

### ACKNOWLEDGMENT OF U.S. GOVERNMENT SUPPORT

This invention was made with U.S. Government support under Research Grant GM-25874 and GM-57840 awarded by the National Institutes of Health. The U.S. Government has certain rights in this invention.

### FIELD OF THE INVENTION

The present invention relates generally to the fields of genetics and cellular and molecular biology, electronics, and nanotechnology. More particularly, the invention relates to amyloid or fibril-forming proteins and the genes that encode them, and especially to prion-like proteins and protein domains and the genes that encode them. The invention further relates to fibril-forming proteins that have been genetically or chemically modified to create fibrils that as electrical conductors, fuses, and electronic circuits.

### DESCRIPTION OF RELATED ART

Nanometer-scale structures are of great interest as potential building blocks for future electronic devices. One significant challenge is the construction of nanowires to enable the electrical connection of such structures. Biomolecules may provide a solution to the difficulty of manufacturing wires at this scale because they naturally exist in the nanometer size range. Biomolecules that self-assemble have the potential to individually pattern into structures to aid the mass production of nanostructures.

The intrinsic properties of biomolecules are generally unsuitable for conducting electrical currents; therefore they are usually combined with an inorganic compound that acts as a conductor. This conductivity is achieved through a hierarchical assembly process where the first step is to form a regular scaffold by using biological molecules followed by a second step where the inorganic components are guided to aggregate selectively along the scaffold.

The first biomolecular templates used for microstructures were phospholipid tubules (Schnur, J. M., et al., Thin Solid Films, 152: 181-206 (1987)), and since then other self-assembling rod-like structures have been assessed for their strengths and weaknesses as nanostructural templates, including DNA, bacteriophages, and microtubules. These materials have many positive characteristics as nanostructure materials. DNA has good recognition capabilities, mechanical rigidity, and amenability to high-precision processing. Recent studies using DNA as a template for gold plating produced wires with ohmic conductivity [resistance, R=86 Ω and a linear current-voltage (I-V) curve] (Harnack, O., et al., Nanosci. Lett., 2: 919-923 (2002)); however, DNA is unstable under conditions (pH 10-12 and temperatures >60° C.) necessary for industrial metallization. Bacteriophages are expected to have similar chemical and thermal constraints, and they do not readily polymerize to form continuous fibers.

Proteins are an attractive alternative material for the construction of nanostructures. Their physical size is appropriate and they are capable of many types of highly specific interactions; indeed, as many as 93,000 different protein-protein interactions have been predicted in yeast (Begley, T. J., et al., Mol. Cancer Res., 1: 103-112 (2002); Uetz, P., et al. Nature, 403: 623-627 (2000); Marcotte, E., et al., Nature, 402: 83-86 (1999)). Moreover, proteins provide an extraordinary array of functionalities that could potentially be coupled to electronic circuitry in the building of nanoscale devices. Protein tubules have the advantage of a high degree of stiffness and greater stability than DNA. In addition they exhibit good adsorption to technical substrates like glass, silicon oxide, or gold. Various protein tubules such as microtubules and rhapidosomes (Fritzsche, W., et al., Appl. Phys. Lett., 75: 2854-2856 (1999); Kirsch, R., et al., Thin Solid Films, 305: 248-253 (1997); Pazirandeh, M. & Campbell, J. R., J. Gen. Microbiol., 139: 859-864 (1993)) have been assessed, but all have important limitations such as relatively high resistance once metallized (of the order of 200 kΩ) (Fritzsche, W., et al., supra), morphology that cannot withstand metallization under industrial conditions, or undesired aggregation once metallized (Kirsch, R., et al., supra). Therefore, there is a need to explore alternative biomaterials.

Prions (protein infectious particles) have been implicated in both human and animal spongiform encephalopathies, including Creutzfeldt-Jakob Disease, kuru, Gerstmann-Strassler-Scheinker Disease, and fatal familial insomnia in humans; the recently-publicized “mad cow disease” in bovines; “scrapie,” which afflicts sheep and goats; transmissible mink encephalopathy; chronic wasting disease of mule, deer, and elk; and feline spongiform encephalopathy. See generally S. Prusiner et al., Cell, 93: 337-348 (1998); S. Prusiner, Science, 278:245-251 (1997); and A. Horwich and J. Weissman, Cell, 89: 499-510 (1997). A currently-accepted theory is that a prion protein (PrP) can exist in at least two conformational states: a normal, soluble cellular form (PrP^{C}) containing little β-sheet structure; and a “scrapie” form (PrP^{Sc}) characterized by significant β-sheet structure, insolubility, and resistance to proteases. Prion particles comprise multimers of the PrP^{Sc} form. Prion formation has been compared and contrasted to amyloid fibril formation that has been observed in other disease states, such as Alzheimer's disease. See J. Harper & P. Lansbury, Annu. Rev. Biochem, 66: 385-407 (1997). More generally, the prion protein has been loosely classified (despite “some significant differences”) as one of at least sixteen known human amyloidogenic proteins that, in an altered conformation, assemble into a fibril-like structure. See J. W. Kelly, Curr. Opin. Struct. Biol., 6: 11-17 (1996), incorporated herein by reference.

There is growing patent and journal literature relating to scientists efforts to develop diagnostic, therapeutic, and prophylactic advances in the area of prion disease. For example, Fishleigh et al., U.S. Pat. No. 5,773,572 describes synthetic peptides that have at least one antigenic site of a prion protein, and suggest using such peptides to raise antibodies and to create vaccines. Prusiner et al., U.S. Pat. No. 5,750,361 describes prion protein peptides having at least one α-helical domain and forming a random coil conformation in aqueous medium, and suggests using such a peptide to assay for the scrapie form of prion protein (PrP^{Sc}).

Weiss et al., J. Virology, 69(8): 4776-83 (1995) state that isolation of PrP^{C} from organisms has been a time-consuming and labor-intensive process. The authors purport to describe the synthesis of Syrian golden hamster prion protein as a fusion with glutathione S-transferase (GST) to enhance solubility and stability of PrP^{C}, and the release of PrP^{C} from the fusion protein via thrombin cleavage. The authors report that only the cellular isoform PrP^{C}, and not the infectious PrP^{Sc} isoform, was produced. [See also Volkel et al., Eur. J. Biochem, 251:462-471 (1998); Meeker et al., Proteins: Structure, Function, and Genetics, 30: 381-387 (1998) (Describing system to overexpress a fusion between the small, minimally soluble serum amyloid A protein and the bacterial enzyme Staphylococcal nuclease; and Zahn et al., FEBS Lett., 417(3): 400-404 (1997) (reporting expression of human PrP proteins fused to a histidine tail to facilitate refolding).]

Prusiner et al., U.S. Pat. Nos. 5,792,901, 5,789,655, and 5,763,740 describe a transgenic mouse comprising a prion protein gene that includes codons from a PrP gene that is native to a different host organism, such as humans, and suggest uses of such mice for prion disease research. The '655 patent teaches to incorporate “a strong epitope tag” in the PrP nucleotide sequence to permit differentiation of PrP protein conformations using an antibody to the epitope. The patents describing these native, mutated, and chimeric PrP gene and protein sequences are incorporated herein by reference. Mouthon et al., Mol. Cell. Neurosci., 11(3):127-133 (1998) report using a fusion between a putative nuclear localization signal of PrP and a green fluorescent protein to study targeting of the protein to the nuclear compartment.

Weissmann et al., U.S. Pat. No. 5,698,763, describes a transgenic mouse in which the PrP gene has been disrupted by homologous recombination, allegedly rendering the mouse non-susceptible to spongiform encephalopathies. Use of PrP anti-sense oligonucleotides to treat non-transgenic animals suffering from an incipient spongiform encephalopathy also is suggested.

Cashman et al., International Publication No. WO 97/45746, purports to describe prion protein binding proteins and uses thereof, e.g., to detect and treat prion-related diseases or to decontaminate samples known to contain or suspected of containing prion proteins. The authors also purport to describe a fusion protein having a PrP portion and an alkaline phosphatase portion, for use as an affinity reagent for labeling, detection, identification, or quantitation of PrP binding proteins or PrP^{Sc}'s in a biological sample, or for use to facilitate the affinity purification of PRP binding proteins.

In addition, there has been significant research in recent years concerning the biology of prion-like elements in yeast. [See, e.g., V. Kushnirov and M. Ter-Avanesyan, Cell, 94: 13-16 (1998); S. Lindquist, Cell, 89: 495-498 (1997); DePace et al., Cell, 93: 1241-1252 (1998); and R. Wickner, Annu. Rev. Genet., 30:109-139 (1996) (all incorporated herein by reference).] Although the two yeast prion-like elements that have been extensively studied do not spread from cell to cell (except during mating or from mother-to-daughter cell) and do not kill the cells harboring them, as has been observed in the case of mammalian PrP prion diseases, certain heritable yeast phenotypes exist that display a very “prion-like” character. The phenotypes appear to arise as the result of the ability of a “normal” yeast protein that has acquired an abnormal conformation to influence other proteins of the same type to adopt the same conformation. Such phenotypes include the [PSI⁺] phenotype, which enhances the suppression of nonsense codons, and the [URE3] phenotype, which interferes with the nitrogen-mediated repression of certain catabolic enzymes. Both phenotypes exhibit cytoplasmic inheritance by daughter cells from a mother cell and are passed to a mating partner of a [PSI⁺] or [URE3] cell.

Yeast organisms present, in many respects, far easier systems than mammals in which to study genotype and phenotype relationships, and the study of the [PSI⁺] and [URE3] phenotypes in yeast has provided significant valuable information regarding prion biology. Studies have implicated the Sup35 subunit of the yeast translation termination factor and the Ure2 protein that antagonizes the action of a nitrogen-regulated transcription activator in the [PSI⁺] and [URE3] phenotypes, respectively. In both of these proteins, the above-stated “normal” biological functions reside in the carboxy-terminal domains, whereas the dispensable, amino-terminal domains have unusual compositions rich in asparagine and glutamine residues.

It is the amino-terminal domains of these proteins (e.g., no more than about residues 2-113 of Sup35 and about residues 1-65 of Ure2) that have been implicated in conferring the [PSI⁺] and [URE3] phenotypes in a prion-like manner. King et al., Proc. Natl Acad Sci USA, 94:6618-6622 (1997), purportedly expressed the N-terminal 114 residues of SUP35 (with a cleavable polyhistidine tag for purification) and reported that this peptide spontaneously aggregates to form thin filaments showing a β-sheet-type circular dichroism in vitro. Deletion of the amino termini of Sup35 and Ure2 in yeast eliminates the [PSI⁺] and [URE3] phenotypes, respectively. In contrast, over-expression of these proteins, or of their amino-terminal. fragments, can induce the [PSI⁺] or [URE3] phenotype de novo. Once cells have acquired the [PSI⁺] or [URE3] phenotype in this manner, they continue to pass the trait to their progeny, even after the plasmid containing the over-expressed element is lost. [See Derkatch et al., Genetics, 144:1375-1386 (1996).]

Interestingly, the Sup35 protein contains similarities to mammalian PrP proteins in that Sup35 is soluble in [psi−] strains but prone to aggregate into insoluble, protease-resistant aggregates in [PSI⁺] strains. In experiments using a fusion between the Sup35 amino terminus and green fluorescent protein (GFP, a protein that fluoresces green on exposure to blue light), it has been shown that the fusion protein is freely distributed in [psi−] cells but aggregated in [PSI⁺] cells. See, e.g., Glover et al., Cell, 89: 811-819 (1997); and Patino et al., Science, 273: 622-626 (1997). Chaperone proteins or “heat shock proteins,” such as the protein Hsp104 in yeast, have been implicated in the conformational conversion of Sup35 protein that is associated with the [PSI⁺] phenotype [see, e.g., J. Glover and S. Lindquist, Cell, 94: 73-82 (1998); V. Kushnirov and M. Ter-Avanesyan, Cell, 94:13-16 (1998); Y. O. Chernoff et al., Science, 268: 880-883 (1995)], and may be implicated in the conformational conversion of PrP. See, e.g., E. Schirmer and S. Lindquist, Proc. Natl. Acad. Sci. USA, 94:13932-13937 (1997); S. DebBurman et al., Proc. Natl. Acad. Sci. USA, 94:13938-13943 (1997).

As the foregoing discussion of literature indicates, there has been significant investigation into the biology of mammalian prions and prion-like yeast proteins for the purposes of developing a basic understanding of prion biology and developing effective measures for diagnosing, treating, and preventing mammalian prion diseases. Practical applications, including taking advantage of the structural characteristics and self-aggregating properties of prions and prion-like proteins, in addition to the immediate medical implications of diagnosing, treating, and preventing spdngiform encephalopathies and other amyloid diseases, is lacking.

### SUMMARY OF THE INVENTION

The present invention relates to materials and methods involving prion-like fibers. For example, embodiments of the invention are directed to nanowires, fuses, circuits, and semiconductors constructed using modified prion-like elements as a scaffold, as well as methods of making and using them.

In one embodiment of the invention, an electrical conductor is provided comprising a fibril having a first location separated from a second location and an electrically conductive material disposed on the fibril between the first location and second location to conduct electricity along the fibril from the first location to the second location. The locations can be, but need not be, the ends of the fibril. In many practical applications, the first location may correspond with a contact beteen the electrical conductor and one element of an electrical circuit, and the second location may correspond to a contact with a second element of the circuit. In a preferred variation, the fibril used to make the electrical conductor comprises polypeptide subunits coalesced into an ordered aggregate, as described herein in detail.

Compared to other biological materials that have been contemplated for use in nanodevices, the fibrils described for use herein (e.g., for making electrical conductors) are characterized by chemical and thermal stability. In particular, the fibrils comprise polymers of polypeptide monomers which, as described below in detail, may exist in a soluble state or an aggregated fibrous state. For the purposes of this invention, a fibril that is characterized by chemical and thermal “stability” if it retains its fiber state for at least 60 minutes under conditions that may be encountered in industrial manufacturing processes and have a tendency to denature at least some proteins, nucleic acids, or other biological polymers. Exemplary conditions include elevated temperatures, extreme acidic or basic conditions, the presence of chemical denaturants, elevated salt conditions, and the presence of organic solvents. For example, fibrils for use in manufacturing an electrical conductor of the present invention preferably are chemically stable in the presence of:
denaturants such as urea (0-2M, more preferably 0-4M, more preferably 0-6M, more preferably 0-8 M) or guanidiniumchloride (0-1M, more preferably 0-2 M);
salt solutions such as 0-1M or more preferably 0-2.5 M NaCl, KCl, sodium phosphate, or other halide salts;
industrial acids (e.g., aqueous solutions with pH between 4 and 7, or more preferably 3 and 7, more preferably 2 and 7, and more preferably 1-7 or 0.1-7;
basic solutions with pH in the range of 7-9, or more preferably 7-10 or 7-11 or 7-12 or 7-13;
organic solvents such as 100% ethanol;
extreme cold such as temperatures between 0-10° C., more preferably −10 to 0° C., −20 to 0° C., −30 to 0° C., −40 to 0° C., −50 to 0° C., −60 to 0° C., −70 to ° C., or −80 to 0° C.;
heat such as temperatures between 50-60° C., and more preferably 50-70° C., 50-80° C., 50-90° C., 50-98° C., or 50-100° C.;
more generally, temperature ranges spanning both extreme cold and heat, e.g., thermal stability from −80° C. to 98° C. or any subranges thereof.

The techniques described herein can be used to make electrical conductors in a wide range of lengths and diameters. For example, electrical conductors may range in length from 0.05 to 10,000 μm in length, with every discrete length and range of lengths therebetween specifically contemplated, such as lengths of 0.06, 0.1, 0.2, 0.5, 0.8, 1, 10, 50, 100, 200 to 300 μm or more. Similarly, fibers may range in diameter from 1, 5, 9, 10, 20, 50, 75, 100, 150 to 200 nm, 300 nm, 400 nm, or 500 nm or more, with every diameter therebetween specifically contemplated as an embodiment of the invention. Diameter is influenced first by the diameter of the protein fibril used to make an electrical conductor, and second, by the amount ant thickness of electrically conductive material disposed on its surface. In one embodiment, the aforementioned electrical conductor is provided wherein the electrical conductor is characterized by a length of 60 nm to 300 μm, and a diameter of 9 nm to 200 nm.

In another embodiment, the aforementioned electrical conductor is provided wherein at least one of the polypeptide subunits comprises a SCHAG amino acid sequence. Thus, the number of SCHAG amino acid sequences comprising an electrical conductor of the present invention can represent 0, 5, 10, 20, 30, 40, 50, 60, 70, 80, 90, or 100% of the total polypeptide subunits in the electrical conductor. In a preferred embodiment, 90-100% of the polypeptide subunits comprise a SCHAG amino acid sequence.

In one embodiment of the invention, the aforementioned electrical conductor is provided wherein the SCHAG amino acid sequence includes at least one amino acid residue having a reactive amino acid side chain. It is possible that the SCHAG amino acid sequence, although containing at least one amino acid with a reactive amino acid side chain at the primary structure level, does not contain an amino acid with a reactive amino acid side chain that is surface exposed at the tertiary and/or quaternary structure level (e.g., when associated with fibrils). Accordingly, another embodiment of the invention provides the aforementioned electrical conductor wherein the SCHAG amino acid sequence includes at least one substitution of an amino acid residue having a reactive amino acid side chain.

Similarly, the number of amino acid substitutions may depend on the spatial relationship between the reactive amino acid side chains exposed to the environment and the length between the same or similar ainino acid side chains of neighboring polypeptides in the fibril. Accordingly, a number of amino acid substitutions sufficient to reduce the gaps between amino acids with reactive side chains between neighboring polypeptides of the aforementioned electrical conductor is contemplated, thereby enabling a continuous connection along the length of the electrical conductor. It is also contemplated that the number of amino acid substitutions is inversely proportional to the amount of electrically conductive material required to provide the continuous connection along the length of the electrical conductor.

In a related embodiment, the aforementioned electrical conductor is provided wherein the reactive amino acid side chain is exposed to the environment of the fibril to permit attachment of the electrically conductive material thereto, and wherein the electrically conductive material is attached to the fibril at the reactive amino acid side chain. Similarly, another embodiment of the invention provides the aforementioned electrical conductor wherein the reactive amino acid side chain of the substituted amino acid is exposed to the environment of the fibril to permit attachment of the electrically conductive material thereto, and wherein the electrically conductive material is attached to the fibril at the reactive amino acid side chain.

SCHAG amino acid sequences are rich in asparagine and glutamine residues. Thus, although many different amino acid sequences can comprise a SCHAG sequence, approximately 30% or more of the amino acid residues of SCHAG sequences may comprise asparagines and/or glutamine residues. Accordingly, in another embodiment of the invention, the aforementioned electrical conductor is provided wherein at least 30%, 35%, 40%, 45%, 50%, 60%, or more of the SCHAG amino acid sequence comprises asparagine or glutamine residues.

In another embodiment, the aforementioned electrical conductor is provided wherein the SCHAG amino acid sequence comprises an amino acid sequence at least 60%, 65%, 70%, 75%, 80%, 85%, 90%, 95%, 97.5%, 98%, 99%, or 100% identical to a sequence selected from the group consisting of SEQ ID NOs: 2, 4, 17, 19, 21, 22, 23, 24, 25, 26, 27, 28, 29, 30, 31, 32, 33, 34, 35, 36, 46, 47, and 50 and aggregation domain fragments thereof. Aggregation domain fragments are those fragments of the aforementioned sequences which contain enough of the original sequence to self-aggregate into fibers as described herein.

In yet another embodiment, the aforementioned electrical conductor is provided wherein the SCHAG amino acid sequence is selected from the group consisting of: a) an amino acid sequence that is at least 60%, 65%, 70%, 75%, 80%, 85%, 90%, 95%, 97.5%, 98%, 99% or 100% identical to amino acids 2 to 113 of SEQ ID NO: 2; and b) an amino acid sequence that is at least 60%, 65%, 70%, 75%, 80%, 85%, 90%, 95%, 97.5%, 98%, or 99% or 100% identical to amino acids 2 to 253 of SEQ ID NO: 2. In a related embodiment, the aforementioned electrical conductor is provided wherein the SCHAG amino acid sequence comprises at least one substitution of an amino acid residue having a reactive amino acid side chain and wherein the reactive amino acid side chain is exposed to the environment of the fibril to permit subsequent attachment of an electrically conductive material thereto.

As exemplified herein, specific amino acid sequences and amino acid substitutions are contemplated by the present invention. In one embodiment, the aforementioned electrical conductor is provided wherein the SCHAG amino acid sequence comprises the amino acid sequence of SEQ ID NO: 2, with the proviso that amino acid 184 of SEQ ID NO: 2 has been substituted for by an amino acid selected from the group consisting of cysteine, lysine, tyrosine, glutamate, aspartate, and arginine. In another embodiment, the aforementioned electrical conductor is provided wherein the SCHAG amino acid sequence comprises the amino acid sequence of SEQ ID NO: 2, with the proviso that amino acid 2 of SEQ ID NO: 2 has been substituted for by an amino acid selected from the group consisting of cysteine, lysine, tyrosine, glutamate, aspartate, and arginine.

Electrically conductive materials contemplated by the present invention include, but are not limited to, materials that comprise metal atoms and semiconductor materials. Thus, in one embodiment of the invention, the aforementioned electrical conductor is provided wherein the electrically conductive material comprises a material selected from the group consisting of a metal atom or a semiconductor material. Exemplary materials that comprise metal atoms are pure metals and metal alloys, inorganic compounds that contain metals, and organometallic compounds and complexes comprised of one or more metal atoms attached to or complexed with an organic compound that can form a covelent bond with a polypeptide. Any conducting metal atom is suitable for practicing the invention, including but not limited to gold, silver, nickel, copper, platinum, aluminum, gallium, palladium, iridium, rhodium, tungsten, titanium, zinc, tin, alloys comprising the same, and combinations thereof. Additional metal atoms are also contemplated. The present invention further provides an electrical conductor wherein the semiconductor material is selected from the group consisting of GaAs, ZnS, CdS, InP and Si.

In one embodiment of the invention, the aforementioned electrical conductor is provided wherein the fibril is gold-toned. It is contemplated by the present invention that an electrical conductor described herein may possess a range of resistances from close to 0 ohms to 5000 ohms and every value in between. For example, resistances may range from 1, 5, 10, 20, 50, 75, 100, 150, 200, 250, 500, or 1000 Ω. In still another embodiment, the aforementioned electrical conductor is provided wherein the fibril is characterized by a resistance range of 0-100 Ω and linear I-V curves at useful power levels. Further, an electrical conductor is provided wherein the fibril is characterized by a resistance range of 0-100 Ω and linear I-V curves between 0 to 0.3×10⁻⁶ A and between 0-30×10⁻⁶ V.

A related aspect of the present invention is a method of making electrical conductors described herein, and methods of making electrical circuits, fuses, or devices comprising the electrical conductors.

For example, in one embodiment, a method of making an electrical conductor is provided comprising steps of: (a) making a fibril with first and second separated locations; and (b) disposing on the fibril an electrically conductive material in an amount effective to conduct electricity along the fibril from the first location to the second location.

Procedures for making the fibril (step (a)) are described below in detail. For example, such procedures comprise providing a solution or suspension of polypeptides that have the ability to coalesce into ordered aggregates, and incubating the solution or suspension under conditions to form fibrils from the polypeptides. A number of physical and chemical variations of such procedures are contemplated. In one embodiment, the method comprises rotating the solution or suspension to increase turbulence and surface area, thereby promoting fibril formation. In a preferred variation, the fiber formation further comprises contacting the fibrils with additional soluble or suspended polypeptide under conditions to extend the length of the fibrils.

The step (b) of disposing electrically conductive material can be performed in any manner by which an electrical conductor such as a metal can be disposed onto a fibril, such as chemical attachment, plating techniques, vapor deposition, combinations thereof, and the like. In one embodiment, step (b) comprises disposing a substrate on the fibril, and disposing a first electrically conductive material on the substrate. The substrate serves as a linker between the fibril and the first electrically conductive material, although the substrate can itself have electrical conducting properties. Thus, in one variation, the disposing the substrate comprises attaching a compound comprising a metal atom to a reactive amino acid side chain of a polypeptide in the fibril. For instance, the substrate optionally comprises gold particles with surface-accessible cross-linking groups. For example, a substrate exemplified herein is Nanogold, an organic, gold-atom containing compound which contains gold atoms and can contribute to electrical conducting properties, and which was attached to exposed cysteine residues of a prion fibril. The Nanogold served as sites for subsequent attachment of silver and/or gold attachment. In a related embodiment, a second electrically conductive material is disposed on the first electrically conductive material.

As described herein, various electrically conductive materials are contemplated for use with the electrical conductors of the present invention. In one embodiment, the aforementioned method is provided wherein the disposing the first electrically conductive material comprises attaching a compound comprising a metal atom to the substrate. Further, the aforementioned method is provided wherein the first electrically conductive material comprises silver ions. In yet another embodiment, the aforementioned method is provided wherein the disposing the second electrically conductive material comprises attaching a compound comprising a metal atom to the first electrically conductive material. In still another embodiment, the aforementioned method is provided wherein the second electrically conductive material comprises gold ions.

In a related embodiment, the aforementioned method is provided wherein the substrate comprises gold particles with surface-accessible cross-linking groups, the first electrically conductive material comprises silver ions, and the second electrically conductive material comprises gold ions. In a related embodiment, the aforementioned method is provided wherein the fibril is characterized by a resistance in the range of 0-100 Ω and a linear current-voltage (I-V) curve.

In still another aspect, the invention includes all variety of electrical devices that can be synthesized with an electrical conductor of the invention. Such devices include everything from nanoscale wires, wires attached to substrates, fuses, circuits, and the like to larger and more complicated devices such as microchips, computers, consumer electronics, medical devices, laboratory tools, and the like that comprise electrical conductors, fuses, or circuits of the invention.

For example, in one embodiment, a fuse is provided comprising an electrical conductor, a first electrode attached to the first position, and a second electrode attached to the second position, wherein the electrical conductor electrically connects the first electrode to the second electrode. In a preferred variation of the fuse, the electrical conductor is constructed to fail to conduct electricity when exposed to an electrical current above a first amount, which can be described as the failure amount or overload amount of power. By “first amount” is simply meant an amount of electrical power (current×voltage) above which a fuse is designed to fail. In one variation, the electrical conductor destructs when exposed to an electric current above the first amount, thereby eliminating electrical conductivity across the fuse.

In another embodiment of the present invention, an electrical circuit is provided comprising a source of electricity, one or more circuit elements, and electrical conductors disposed between the source of electricity and the one or more circuit elements, wherein at least one of the electrical conductors is an electrical conductor of the invention. For example, the electrical conductor comprises a fibril and an electrically conductive material disposed on the fibril to conduct electricity along the fibril from a first position on the fibril such as the source of electricity to a second position on the fibril, such as one of the circuit elements. The electrical conductor also may be disposed between two circuit elements. Exemplary circuit elements includes any circuit component selected from the group consisting of a capacitor, an inductor, a resistor, an integrated circuit, an oscillator, a transistor, a diode, a switch, and a fuse. The one or more circuit elements may be passive circuit elements, active circuit elements, or combinations thereof.

The present invention is also directed to employing unique features of prion biology in a practical context beyond fundamental prion research and applied research directed to the development of diagnostic, therapeutic, and prophylactic treatments of mammalian prion diseases (although aspects of the invention have utility in such contexts also). Likewise, the present invention also relates to the construction of novel prion-like elements that can change the phenotype of a cell in a beneficial way.

In one aspect, the invention provides a polynucleotide comprising a nucleotide sequence that encodes a chimeric polypeptide, the polynucleotide comprising: a nucleotide sequence encoding at least one SCHAG amino acid sequence fused in frame with a nucleotide sequence encoding at least one polypeptide of interest other than a marker protein, or a glutathione S-transferase (GST) protein, or a staphylococcal nuclease protein. In a preferred embodiment, the polynucleotide has been purified and isolated. In another preferred embodiment, the polynucleotide is stably transformed or transfected into a living cell.

By “chimeric polypeptide” is meant a polypeptide comprising at least two distinct polypeptide segments (domains) that do not naturally occur together as a single protein. In preferred embodiments, each domain contributes a distinct and useful property to the polypeptide. Polynucleotides that encode chimeric polypeptides can be constructed using conventional recombinant DNA technology to synthesize, amplify, and/or isolate polynucleotides encoding the at least two distinct segments, and to ligate them together. See, e.g., Sambrook et al., Molecular Cloning—A Laboratory Manual, Second Ed., Cold Spring Harbor Press (1989); and Ausubel et al., Current Protocols in Molecular Biology, John Wiley & Sons, Inc. (1998); both incorporated herein by reference.

The chimeric polypeptide comprises a SCHAG amino acid sequence as one of its polypeptide segments. By “SCHAG amino acid sequence” is meant any amino acid sequence which, when included as part or all of the amino acid sequence of a protein, can cause the protein to coalesce with like proteins into higher ordered aggregates commonly referred to in scientific literature by terms such as “amyloid,” “amyloid fibers,” “amyloid fibrils,” “fibrils,” or “prions.” In this regard, the term SCHAG is an acronym for Self-Coalesces into Higher-ordered AGgregates. By “higher ordered” is meant an aggregate of at least 25 polypeptide subunits, and is meant to exclude the many proteins that are known to comprise polypeptide dimers, tetramers, or other small numbers of polypeptide subunits in an active complex. The term “higher-ordered aggregate” also is meant to exclude random agglomerations of denatured proteins that can form in non-physiological conditions. [From the term “self-coalesces,” it will be understood that a SCHAG amino acid sequence may be expected to coalesce with identical polypeptides and also with polypeptides having high similarity (e.g., less than 10% sequence divergence) but less than complete identity in the SCHAG sequence.] It will be understood than many proteins that will self-coalesce into higher-ordered aggregates can exist in at least two conformational states, only one of which is typically found in the ordered aggregates or fibrils. The term “self-coalesces” refers to the property of the polypeptide to form ordered aggregates with polypeptides having an identical amino acid sequence under appropriate conditions as taught herein, and is not intended to imply that the coalescing will naturally occur under every concentration or every set of conditions. In fact, data exists suggesting that trans-acting factors, such as chaperone proteins, may be involved in the protein's conformational switching, in vivo.) Aggregates formed by SCHAG polypeptides typically are rich in β-sheet structure, as demonstrated by circular dichroism; bind Congo red dye and give a characteristic spectral shift in polarized light; and are insoluble in water or in solutions mimicking the physiological salt concentrations of the native cells in which the aggregates originate. In preferred embodiments the SCHAG polypeptides self-coalesce to form amyloid fibrils that typically are 5-20 nm in width and display a “cross-β” structure, in which the individual β strands of the component proteins are oriented perpendicular to the axis of the fibril. The SCHAG amino acid sequence may be said to constitute an “amyloidogenic domain” or “fibril-aggregation domain” of a protein because a SCHAG amino sequence confers this self-coalescing property to proteins which include it.

Exemplary SCHAG amino acid sequences include sequences of any naturally occurring protein that has the ability to aggregate into amyloid-type ordered aggregates under physiological conditions, such as inside of a cell. In one preferred embodiment, the SCHAG amino acid sequence includes the sequences of only that portion of the protein responsible for the aggregation behavior. Many such sequences have been identified in humans and other animals, including amyloid β protein (residues 1-40, 1-41, 1-42, or 1-43), associated with Alzheimer's disease; immunoglobulin light chain fragments, associated with primary systemic amyloidosis; serum amyloid A fragments, associated with secondary systemic amyloidosis; transthyretin and transthyretin fragments, associated with senile systemic amyloidosis and familial amyloid polyneuropathy I; cystatin C fragments, associated with hereditary cerebral amyloid angiopathy; β₂-microglobulin, associated with hemodialysis-related amyloidosis; apolipoprotein A-1 fragments, associated with familial amyloid polyneuropathy III; a 71 amino acid fragment of gelsolin, associated with Finnish hereditary systemic amyloidosis; islet amyloid polypeptide fragments, associated with Type II diabetes; calcitonin fragments, associated with medullary carcinoma of the thyroid; prion protein and fragments thereof, associated with spongiform encephalopathies; atrial natriuretic factor, associated with atrial amyloidosis; lysozyme and lysozyme fragments, associated with hereditary non-neuropathic systemic amyloidosis; insulin, associated with injection-localized amyloidosis; and fibrinogen fragments, associated with hereditary renal amyloidosis. See J. W. Kelly, Curr. Op. Struct. Biol., 6: 11-17 (1996), incorporated herein by reference. In addition, several other SCHAG amino acid sequences of yeast and fungal origin are described in detail below. Also, the Examples below set forth in detail how to use the SCHAG sequences specifically identified herein or elsewhere in the literature to screen databases or genomes for additional naturally occurring SCHAG amino acid sequences. The Examples also provide assays to screen candidate SCHAG sequences for prion-like properties. In addition, the Examples provide assays to rapidly screen random DNA fragments to determine whether they encode a SCHAG amino acid sequence. Such screening assays are themselves considered aspects of the invention.

In addition, SCHAG amino acid sequences include those sequences derived from naturally occurring SCHAG amino acid sequences by addition, deletion, or substitution of one or more amino acids from the naturally occurring SCHAG amino acid sequences. Detailed guidelines for modifying SCHAG amino acid sequences to produce synthetic SCHAG amino acid sequences are described below. Modifications that introduce conservative substitutions are specifically contemplated for creating SCHAG amino acid sequences that are equivalent to naturally occurring sequences. By “conservative amino acid substitution” is meant substitution of an amino acid with an amino acid having a side chain of a similar chemical character. Similar amino acids for making conservative substitutions include those having an acidic side chain (glutamic acid, aspartic acid); a basic side chain (arginine, lysine, histidine); a polar amide side chain (glutamine, asparagine); a hydrophobic, aliphatic side chain (leucine, isoleucine, valine, alanine, glycine); an aromatic side chain (phenylalanine, tryptophan, tyrosine); a small side chain (glycine, alanine, serine, threonine, methionine); or an aliphatic hydroxyl side chain (serine, threonine). Alternatively, similar amino acids for making conservative substitutions can be grouped into three categories based on the identity of the side chains. The first group includes glutamic acid, aspartic acid, arginine, lysine, histidine, which all have charged side chains; the second group includes glycine, serine, threonine, cysteine, tyrosine, glutamine, asparagine; and the third group includes leucine, isoleucine, valine, alanine, proline, phenylalanine, tryptophan, methionine, as described in Zubay, G., Biochemistry, third edition, Wm. C. Brown Publishers (1993).

Also contemplated are modifications to naturally occurring SCHAG amino acid sequences that result in addition or substitution of polar residues (especially glutamine and asparagine, but also serine and tyrosine) into the amino acid sequence. Certain naturally occurring SCHAG amino acid sequences are characterized by short, sometimes imperfect repeat sequences of, e.g., 5-12 residues. Modifications that result in substantial duplication of such repetitive oligomers are specifically contemplated for creating SCHAG amino acid sequences, too.

In another variation of the invention, the SCHAG amino acid sequence is encoded by a polynucleotide that hybridizes to any of the nucleotide sequences of the invention; or the non-coding strands complementary to these sequences, under the following exemplary moderately stringent hybridization conditions:
(a) hybridization for 16 hours at 42° C. in an aqueous hybridization solution comprising 50% formamide, 1% SDS, 1 M NaCl, 10% Dextran sulphate; and
(b) washing 2 times for 30 minutes at 60° C. in an aqueous wash solution comprising 0.1% SSC, 1% SDS. Alternatively, highly stringent conditions include washes at 68° C.

Also provided are purified and isolated polynucleotide comprising a nucleotide sequence that encodes at least one SCHAG amino acid sequence, wherein the SCHAG-encoding portion of the polynucleotide is at least about 99%, at least about 98%, at least about 95%, at least about 90%, at least about 85%, at least about 80%, at least about 75%, or at least about 70% identical over its full length to one of the nucleotide sequences of the invention. Methods of screening for natural or artificial sequences for SCHAG properties are also described elsewhere herein.

A preferred category of SCHAG amino acid sequences are prion aggregation domains from prion proteins. The term “prion-aggregation domain” is intended to define a subset of SCHAG amino acid sequences that can exist in at least two conformational states, only one of which is typically found in the aggregated state. In one conformational state, proteins comprising the prion-aggregation domain or fused to the prion-aggregation domain perform their normal function in a cell, and in another conformational state, the native proteins form aggregates (prions) that phenotypically alter the cell, perhaps by sequestering the protein away from its normal site of subcellular activity, or by disrupting the conformation of an active domain of the protein, or by changing its activity state, or bay acquiring a new activity upon aggregation, or perhaps merely by virtue of a detrimental effect on the cell of the aggregate itself. A hallmark feature of prion-aggregation domains is that the phenotypic alteration that is associated with prion formation is heritable and/or transmissible: prions are passed from mother to daughter cell or to mating partners in organisms such as in the case of yeast Sup35, and Ure2 prions, perpetuating the [PSI⁺] or [URE3] prion phenotypes, or the prions are transmitted in an infectious manner in organisms such as in the case of PrP prions in mammals, leading to transmissible spongiform encephalopathies. This defining characteristic of prions is attributable, at least in part, to the fact that the aggregated prion protein is able to promote the rearrangement of unaggregated protein into the aggregated conformation (although chaperone-type proteins or other trans-acting factors in the cell may also assist with this conformational change). It is likewise a feature of prion-aggregation domains that over-production of proteins comprising these domains increases the frequency with which the prion conformation and phenotype spontaneously arises in cells.

Prion aggregation amino acid sequences comprising amino terminal sequences derived from yeast or fungal Sup35 proteins, Ure2 proteins, or the carboxy terminal sequences derived from yeast Rnq1 proteins are among those that are highly preferred. Referring to the S. cerevisiae Sup35 amino acid sequence set forth in SEQ ID NO: 2, experiments have shown that no more than amino acids 2-113 (the N domain) of that sequence are required to confer some prion aggregation properties to a protein, although inclusion of the charged “M” (middle) region immediately downstream of these residues, e.g., thru residue 253, is preferred in some embodiments. The N domain alone is very amyloidogenic and immediately aggregates into fibers, even in the presence of 2 M urea, a phenomenon that is desirable in embodiments of the invention where formation of stable fibrils of chimeric polypeptides is preferred. When the N domain is fused to the highly charged M domain, fiber formation proceeds in a slower, more orderly way. The M domain is postulated to shift the equilibrium to permit greater “switchability” between aggregated and soluble forms, and is preferably included where phenotypic switching is desirable. Referring to the S. cerevisiae Ure2 amino acid sequence set forth in SEQ ID NO: 4, experiments have shown that no more than amino acids 2-65 of that sequence are required to confer prion aggregation activity to a protein. Referring to the S. cerevisiae Rnq1 amino acid sequence set forth in SEQ ID NO: 50, experiments have shown that no more than amino acids 153-405 of that sequence are required to confer prion aggregation activity to a protein. Moreover, sequences differing from the native sequences by the addition, deletion, or substitution of 1, 2, 3, 4, 5, 6, 7, 8, 9, 10, 11, 12, 13, 14, 15, 16, 17, 18, 19, 20, or more amino acids, especially the addition or substitution of additional glutamine or asparagine residues, but which retain the properties of prion-aggregation domains as described in the preceding paragraph, are contemplated. Also, orthologs (corresponding proteins or prion aggregation domains thereof from different species) comprise an additional genus of preferred sequences (Kushinov et al., Yeast 6:461-472 (1990); Chernoff et al., Mol Microbiol 35:865-876 (2000); Santoso et al., Cell 100:277-288 (2000); and Kushinov et al., EMBO J 19:324-31 (2000)). By way of example, Sup35 amino acid sequences from Pichia pinus and Candida albicans are set forth in Genbank Accession Nos. X56910 (SEQ ID NO: 46) and AF 020554 (SEQ ID NO: 47), respectively. Polypeptides of the invention include polypeptides that are encoded by polynucleotides that hybridize under stringent, preferably highly stringent conditions, to the polynucleotide sequences of the invention, or the non-coding strand thereof. Polypeptides of the invention also include polypeptides that are at least about 99%, at least about 98%, at least about 95%, at least about 90%, at least about 85%, at least about 80%, at least about 75%, or at least about 70% identical to one of SCHAG amino acid sequences of the invention.

As set forth above, in some aspects of the invention, the nucleotide sequence encoding the SCHAG amino acid sequence of the polypeptide is fused in frame with a nucleotide sequence encoding at least one polypeptide of interest. By “in frame” is meant that when the nucleotide is transformed into a host cell, the cell can transcribe and translate the nucleotide sequence into a single polypeptide comprising both the SCHAG amino acid sequence and the at least one polypeptide of interest. It is contemplated that the nucleotide sequences can be joined directly; or that the nucleotide sequences can be separated by additional codons. Such additional codons may encode an endopeptidase recognition sequence or a chemical recognition sequence or the like, to permit enzymatic or chemical cleavage of the SCHAG amino acid sequence from the polypeptide of interest, to permit isolation of the polypeptide of interest. Preferred recognition sequences are sequences that are not found in the polypeptide of interest, so that the polypeptide of interest is not internally cleaved during such isolation procedures. It will be understood that modification of the polypeptide of interest to eliminate internal recognition sequences may be desirable to facilitate subsequent cleavage from the SCHAG amino acid sequence. Suitable enzymatic cleavage sites include: the amino acid sequences -(Asp)ₙ-Lys-, wherein n signifies 2, 3 or 4, recognized by the protease enterokinase; -Ile-Glu-Gly-Arg-, recognized by coagulation factor Xₐ; an arginine residue or a lysine residue cleaved by trypsin; a lysine residue cleaved by lysyl endopeptidase; a glutamine residue cleaved by V8 protease, and a glu-asn-leu-tyr-phe-gln-gly site recognized by the tobacco etch virus (TEV) protease. Suitable chemical cleavage sites include tryptophan residues cleaved by 3-bromo-3-methyl-2-(2-nitrophenylmercapto)-3H-indole; cysteine residues cleaved by 2-nitroso-5-thiocyano benzoic acid; the dipeptides -Asp-Pro- or -Asn-Gly- which can be cleaved by acid and hydroxylamine, respectively; and a methionine residue which is specifically cleaved by cyanogen bromide (CNBr). In another variation, the additional codons comprise self-splicing intein sequences that can be activated, e.g., by adjustments to pH. See Chong et al., Gene, 192:27-281 (1997).

Additional codons also may be included between the sequence encoding the prion aggregation amino acid sequence and the sequence encoding the protein of interest to provide a linker amino acid sequence that serves to spatially separate the SCHAG amino acid sequence from the polypeptide of interest. Such linkers may facilitate the proper folding of the polypeptide of interest, to assure that it retains a desired biological activity even when the protein as a whole has formed aggregates with other proteins containing the SCHAG amino acid sequence. Also, additional codons may be included simply as a result of cloning techniques, such as ligations and restriction endonuclease digestions, and strategic introduction of restriction endonuclease recognition sequences into the polynucleotide.

In still another variation, the additional codons comprise a hydrophilic domain, such as the highly-charged M region of yeast Sup35 protein. While the N domain of Sup35 has proven sufficient in some cases to effect prion-like behavior, suggesting that the M region is not absolutely required in all cases, it is contemplated that the M region or a different peptide that includes hydrophilic amino acid side chains will in some cases be helpful for modulating prion-like character of chimeric peptides of the invention. Without intending to be limited to a particular theory, the highly charged M domain is thought to act as a “solublization” domain involved in modulating the equilibrium between the soluble and the aggregate forms of Sup35, and these properties may be advantageously adapted for other SCHAG sequences.

By “polypeptide of interest” is meant any polypeptide that is of commercial or practical interest and that comprises an amino acid sequence encodable by the codons of the universal genetic code. Exemplary polypeptides of interest include: enzymes that may have utility in chemical, food-processing (e.g., amylases), or other commercial applications; enzymes having utility in biotechnology applications, including DNA and RNA polymerases, endonucleases, exonucleases, peptidases, and other DNA and protein modifying enzymes; polypeptides that are capable of specifically binding to compositions of interest, such as polypeptides that act as intracellular or cell surface receptors for other polypeptides, for steroids, for carbohydrates, or for other biological molecules; polypeptides that comprise at least one antigen binding domain of an antibody, which are useful for isolating that antibody's antigen; polypeptides that comprise the ligand binding domain of a ligand binding protein (e.g., the ligand binding domain of a cell surface receptor); metal binding proteins (e.g., ferritin (apoferritin), metallothioneins, and other metalloproteins), which are useful for isolating/purifying metals from a solution containing them for metal recovery or for remediation of the solution; light-harvesting proteins (e.g., proteins used in photosynthesis that bind pigments); proteins that can spectrally alter light (e.g., proteins that absorb light at one wavelength and emit light at another wavelength); regulatory proteins, such as transcription factors and translation factors; and polypeptides of therapeutic value, such as chemokines, cytokines, interleukins, growth factors, interferons, antibiotics, immunopotentiators and immunosuppressors, and angiogenic or anti-angiogenic peptides.

However, specifically excluded from the scope of the invention are chimeric polynucleotides that have heretofore been described in the literature. For example, excluded from the scope of the invention are polynucleotides encoding a fusion consisting essentially of a SCHAG domain of a characterized protein fused in-frame to only: (1) a marker protein such as a fluorescing protein (e.g., green fluorescent protein or firefly luciferase), an antibiotic resistance-conferring protein, a protein involved in a nutrient metabolic pathway that has been used in the literature for selective growth on incomplete growth media, or a protein (e.g., β-galactosidase, an alkaline phosphatase, or a horseradish peroxidase) involved in a metabolic or enzymatic pathway of a chromogenic or luminescent substrate that results in the production of a detectable chromophore or light signal that has been used in the literature for identification, selection, or quantitation; or (2) a protein (e.g., glutathione S-transferase or Staphylococcal nuclease) that has been used in the literature as a fusion partner for the express purpose of facilitating expression or purification of other proteins. Notwithstanding this exclusion of certain products from the invention, the inventors contemplate novel uses of such specifically excluded products as aspects of the present invention. Moreover, polynucleotides that include a SCHAG sequence, and sequence encoding a polypeptide of interest, and a sequence encoding a marker protein such as green fluorescent protein are considered within the scope of the invention. Also, notwithstanding the above exclusion, polynucleotides that encode polypeptides whose SCHAG properties are described herein for the first time, fused to a marker protein, are considered within the scope of the invention. Also, purified fusion polypeptides that have been described in the literature and examined only in vivo, but never purified, are intended as aspects of the invention. For example, isolated fibers comprising polypeptides encoding a fusion protein consisting of essentially one or more SCHAG sequences fused to a marker protein, e.g., GFP are contemplated. Several such examples are provided in Example 5.

The encoding sequences of the polynucleotide may be in either order, i.e., the SCHAG amino acid encoding sequence may be upstream (5′) or downstream (3′) of the sequence, such that the SCHAG amino acid sequence of the resultant protein is disposed at an amino-terminal or carboxyl-terminal position relative to the protein of interest. In the case of SCHAG amino acid sequences identified or derived from sequences in nature, the encoding sequences preferably are ordered in a manner mimicking the order of the polypeptide from which the SCHAG amino acid sequence was derived. For example, the yeast Sup35 protein has an amino terminal SCHAG domain and a carboxy-terminal domain containing Sup35 translation termination activity. Thus, in embodiments of the invention where the SCHAG amino acid encoding sequence is derived from a Sup35 protein, this sequence preferably is disposed upstream (5′) of the sequence encoding the at least one polypeptide of interest. In embodiments wherein the fibril-aggregation amino acid encoding sequence is derived from the sequence set forth in Genbank Accession No. p25367 (SEQ ID NO: 29) (where the prion-like domain is C-terminal), this sequence is preferably disposed downstream (3′) of the sequence encoding the at least one polypeptide of interest. In an embodiment comprising sequences encoding two or more polypeptides of interest, the SCHAG encoding sequence may be disposed between the two polypeptides of interest.

To the extent that such sequences are not already inherent in the above-described polynucleotides, it will be understood that such polynucleotides preferably further comprise a translation initiation codon fused in frame and upstream (5′) of the encoding sequences, and a translation stop codon fused in frame and downstream (3′) of the encoding sequences. Also, it may be desirable in some embodiments to direct a host cell to secrete the chimeric polypeptide. Thus, it is contemplated that the polynucleotide may further comprise a nucleotide sequence encoding a translation initiation codon and a secretory signal peptide fused in frame and upstream of the encoding sequences.

In preferred embodiments, the polynucleotide of the invention further comprises additional sequences to facilitate and/or control expression in selected host cells. For example, the polynucleotide includes a promoter and/or an enhancer sequence operatively connected upstream (5′) of the encoding sequences, to promoter expression of the encoding sequences in the selected host cell; and/or a polyadenylation signal sequence operatively connected downstream (3′) of the encoding sequences. Since concentration is a factor that may influence the aggregation state of encoded chimeric polypeptides, regulatable (e.g., inducible and repressible) promoters are highly preferred.

To facilitate identification of cells that have been successfully transformed/transfected with the polynucleotide of the invention, the polynucleotide may further include a sequence encoding a selectable marker protein. The selectable marker may be a completely distinct open reading frame on the polynucleotide, such as an open reading frame encoding an antibiotic resistance protein or a protein that facilitates survival in a selective nutrient medium. The selectable marker also may itself be part of the chimeric polypeptide of the invention. In one embodiment, a visual marker such as a fluorescent protein (e.g., green fluorescent protein) is used that is distributed in the cell in a different manner when the protein is in the prion form than when the protein is in the non-prion form. In either case, cells comprising the selectable marker can be sorted, e.g., using techniques such as fluorescence activated cell sorting. Thus, this marker, in addition to permitting selection of transformed or transfected cells, also permits identification of the conformational state of the chimeric polypeptide. In another embodiment, the marker has two components: 1) a function that is changed when the protein is in a prion form and 2) a visual or selectable marker for that function. An example is the glucocorticoid receptor, GR and a reporter gene. GR is a transcription factor that binds to a specific DNA sequence to activate transcription. When this DNA sequence is fused to the coding sequence for an easily detected protein such as β-galactosidase or luciferase GR function can be easily assayed by the induction of the β-galactosidase or luciferase proteins.

Optionally, the polynucleotide of the invention further includes an epitope tag fused in frame with the encoding sequences, which tag is useful to facilitate detection in vivo or in vitro and to facilitate purification of the chimeric polypeptide or of the protein of interest after it has been cleaved from the SCHAG amino acid sequence of the chimeric polypeptide. (An epitope tag alone is not considered to constitute a polypeptide of interest.) A variety of natural or artificial heterologous epitopes are known in the art, including artificial epitopes such as FLAG, Strep, or poly-histidine peptides. FLAG peptides include the sequence Asp-Tyr-Lys-Asp-Asp-Asp-Asp-Lys (SEQ ID NO: 5) or Asp-Tyr-Lys-Asp-Glu-Asp-Asp-Lys (SEQ ID NO: 6). [See generally Brewer, Bioprocess. Technol., 2: 239-266 (1991); Kunz, J. Biol. Chem., 267: 9101-9106 (1992); Brizzard et al., Biotechniques 16: 730-735 (1994); Schafer, Biochem. Biophys. Res. Commun., 207: 708-714 (1995).] The Strep epitope has the sequence Ala-Trp-Arg-His-Pro-Gln-Phe-Gly-Gly (SEQ ID NO: 7). [See Schmidt, J. Chromatography, 676: 337-345 (1994).] Another commonly used artificial epitope is a poly-His sequence having six consecutive histidine residues. Commonly used naturally-occurring epitopes include the influenza virus hemagglutinin sequence Tyr-Pro-Tyr-Asp-Val-Pro-Asp-Tyr-Ala-Ile-Glu-Gly-Arg (SEQ ID NO: 8) and truncations thereof, which is recognized by the monoclonal antibody 12CA5 [Murray et al., Anal. Biochem., 229: 170-179 (1995)] and the sequence (Glu-Gln-Lys-Leu-Leu-Ser-Glu-Glu-Asp-Leu-Asn) (SEQ ID NO: 9) from human c-myc, which is recognized by the monoclonal antibody 9E10 (Manstein et al., Gene, 162: 129-134 (1995)).

In another embodiment, the polynucleotide includes 5′ and 3′ flanking regions that have substantial sequence homology with a region of an organism's genome. Such sequences facilitate introduction of the chimeric gene into the organism's genome by homologous recombination techniques.

In yet another aspect, the invention provides a polynucleotide comprising a nucleotide sequence that encodes a chimeric polypeptide, the chimeric polypeptide comprising an amyloidogenic domain that causes the polypeptide to aggregate with polypeptides sharing an identical or nearly identical domain into ordered aggregates such as fibrils, fused to a domain comprising a polypeptide of interest; wherein the amyloidogenic domain comprises an amyloidogenic amino acid sequence of a naturally occurring protein and further includes a duplication of at least a portion of the naturally occurring amyloidogenic amino acid sequence, the duplication increasing the amyloidogenic affinity of the chimeric polypeptide relative to an identical chimeric polypeptide lacking the duplication. By way of example, if the naturally occurring protein comprises a Sup35 protein of Saccharomyces cerevisiae that is characterized by the partial amino acid sequence PQGGYQQYN (SEQ ID NO: 10), which sequence exists as multiple imperfect repeats, the duplication preferably includes the amino acid sequence PQGGYQQYN and/or an imperfect repeat thereof, such as a repeat wherein one or two residues has been added, deleted, or substituted. An exemplary sequence containing the NM regions of yeast Sup35, with two additional repeat segments, is set forth in SEQ ID NOs: 16 and 17.

In a related aspect, the invention provides a polynucleotide comprising a nucleotide sequence that encodes a chimeric polypeptide, the chimeric polypeptide comprising an amyloidogenic domain that causes the polypeptide to aggregate with identical polypeptides into fibrils, fused to a domain comprising a polypeptide of interest; wherein the amyloidogenic domain comprises amyloidogenic amino acid sequences of at least two naturally occurring amyloidogenic proteins.

In yet another related aspect, the invention provides a polynucleotide comprising a nucleotide sequence of the formula FPBT or FBPT, wherein: B comprises a nucleotide sequence encoding a polypeptide that is encoded by a portion of the genome of the cell; F and T comprise, respectively, 5′ and 3′ flanking sequences adjacent to the sequence encoding B in the genome of the cell; and P comprises a nucleotide sequence encoding a prion-aggregation amino acid sequence, wherein P is fused in frame to B. Using such polynucleotides and conventional homologous recombination techniques [see, e.g., Ausbel et al. (1998), Volume 3, supra], one can perform homologous recombination in a living cell to convert a protein-encoding gene of the cell to a prion gene of the cell, as described in greater detail below. Alternatively, strains can be constructed wherein the endogenous protein-encoding gene is deleted and a prion version of the gene is added back into the cell, either on a plasmid or by integration into the host genome.

The homologous recombination technique is itself intended as an aspect of the invention. For example, the invention provides a method of modifying a living cell to create an inducible and stable phenotypic alteration in the cell, comprising the steps of: transforming a living cell with the polynucleotide described in the preceding paragraph; culturing the cell under conditions that permit homologous recombination between the polynucleotide and the genome of the cell; and selecting a cell in which the polynucleotide has homologously recombined with the genome to create a genomic sequence comprising the formula PB or BP.

More generally, the invention provides a method of modifying a living cell to create an inducible and stable phenotypic alteration in the cell, such as a method comprising steps of: identifying a target polynucleotide sequence in the genome of the cell that encodes a polypeptide of interest; and transforming the cell to substitute for or modify the target sequence, wherein the substitution or modification produces a cell comprising a polynucleotide that encodes a chimeric polypeptide, wherein the chimeric polypeptide comprises a SCHAG amino acid sequence fused in frame with the polypeptide of interest. Such modifications can be performed in several ways, such as (1) homologous recombination as described in the preceding paragraphs; (2) knockout or inactivation of the target sequence followed by introduction of an exogenous chimeric sequence encoding the desired chimeric polypeptide; or (3) targeted introduction of a SCHAG-encoding polynucleotide sequence upstream and in-frame with the target sequence encoding the polypeptide of interest; (4) subsequent cloning or sexual reproduction of such cells; and/or other techniques developed by those in the art.

The foregoing aspects of the invention relate largely to polynucleotides. Also intended as part of the invention are vectors comprising the polynucleotides, and host cells comprising either the polynucleotides or comprising the vectors. Vectors are useful for amplifying the polynucleotides in host cells. Preferred vectors include expression vectors, which contain appropriate control sequences to permit expression of the encoded chimeric protein in a host cell that has been transformed or transfect with the vectors. Both prokaryotic and eukaryotic host cells are contemplated as aspects of the invention. The host cell may be from the same kingdom (prokaryotic, animal, plant, fungi, protista, etc.) as the organism from which the SCHAG amino acid sequence of the polynucleotide was derived, or from a different kingdom. In a preferred embodiment, the host cell is from the same species as the organism from which the SCHAG amino acid sequence of the polynucleotide was derived.

In yet another embodiment, the invention includes a host cell transformed or transfected with at least two polynucleotides encoding chimeric polypeptides according to the invention, wherein the at least two polynucleotides comprise compatible SCHAG amino acid sequences and distinct polypeptides of interest. Such host cells are capable of producing two chimeric polypeptides of the invention, which can be induced in vitro or in vivo to aggregate with each other into higher ordered aggregates. As explained in greater detail below, such aggregates can be advantageously employed in multi-step chemical reactions when the two or more polypeptides of interest each participate in a step of the reaction. Experiments using fluorescence resonance energy transfer (FRET) have demonstrated the efficacy of heterogeneous polypeptide aggregation into co-polymers.

In addition, the chimeric polypeptides encoded by any of the foregoing polynucleotides are intended as an aspect of the invention. Purified polypeptides are preferred, and are obtained using conventional polypeptide purification techniques. For example, the invention provides a chimeric polypeptide comprising: at least one SCHAG amino acid sequence and at least one polypeptide of interest other than a marker protein, a glutathione S-transferase (GST) protein, or a Staphylococcal nuclear protein. As described above, the SCHAG amino acid sequence may be directly linked (via a peptide bond) to the polypeptide of interest, or may be indirectly linked by virtue of the inclusion of an intermediate spacer region, a solubility domain, an epitope to facilitate recognition and purification, and so on.

As explained herein in detail, polypeptides of the invention are capable of existing in a conformation in which the polypeptide coalesces with similar polypeptides into ordered aggregates that may be referred to as “amyloid,” “fibrils,” “prions;” or “prion-like aggregates.” Such ordered aggregates of polypeptides of the invention are intended as an additional aspect of the invention. Such ordered aggregates tend to be insoluble in water or under physiological conditions mimicking a host cell, and consequently can be purified and isolated using standard procedures, including but not limited to centrifugation or filtration. In a preferred embodiment, the SCHAG amino acid sequence is an amino acid sequence that will self-coalesce into ordered “cross-β” fibril structures that are filamentous in character, in which individual β-sheet strands of component chimeric proteins are oriented perpendicular to the axis of the fibril. In a highly preferred embodiment, the polypeptide of interest is disposed radiating away from the fibril core of SCHAG peptide sequences, and retains one or more characteristic biological activities (e.g., binding activities for polypeptides of interest that have specific binding partners; enzymatic activity for polypeptides of interest that are enzymes).

In still another embodiment, the invention provides a composition comprising an ordered aggregate of at least two chimeric polypeptides of the invention, wherein the at least two chimeric polypeptides have compatible SCHAG amino acid sequences and distinct polypeptides of interest. By “compatible” SCHAG amino acid sequences is meant SCHAG amino acid sequences that are either identical or sufficiently similar to permit co-aggregation with each other into higher ordered aggregates. In a preferred embodiment, the two or more polypeptides of interest retain their native biological activity (e.g., binding activity; enzymatic activity) in the ordered aggregate. Such aggregates can be advantageously employed in multi-step chemical reactions, as described in detail below.

The invention further includes methods of making and using polynucleotides and polypeptides of the invention.

For example, the invention provides a method comprising the steps of: transforming or transfecting a cell with a polynucleotide of the invention; and growing the cell under conditions which result in expression of the chimeric polypeptide that is encoded by the polynucleotide in the cell. In a preferred embodiment, the method further includes the step of isolating the chimeric polypeptide from the cell or from growth medium of the cell. In one variation, the method further comprises the step of detaching the SCHAG amino acid sequence of the protein from the polypeptide of interest. As described above in detail, the detachment may be effected with any appropriate means, including chemicals, proteolytic enzymes, self-splicing inteins, or the like. Optionally, the method further includes the step of isolating the protein of interest from the SCHAG amino acid sequence.

In a related embodiment, the invention provides a method of making a protein of interest, comprising the steps of: transforming or transfecting a cell with a polynucleotide, the polynucleotide comprising a nucleotide sequence that encodes a chimeric polypeptide, the chimeric polypeptide comprising an amyloidogenic domain that causes the polypeptide to aggregate with identical polypeptides into higher-ordered aggregates such as fibrils, fused to domain comprising a polypeptide of interest; growing the cell under conditions which result in expression of the chimeric polypeptide in the cell and aggregation of the chimeric polypeptide into fibrils; and isolating the chimeric polypeptide from the cell or from growth medium of the cell. In a preferred embodiment, the isolating step comprises the step of separating the fibrils from soluble proteins of the cell. In a highly preferred embodiment, the method further comprises the steps of proteolytically detaching the amyloidogenic domain of the chimeric protein from the polypeptide of interest; and isolating the polypeptide of interest. Preferably the detached polypeptide of interest maintains one or more of its biological functions, e.g., enzymatic activity, the ability to bind to its ligand, the ability to induce the production of antibodies in a suitable host system, etc.

In yet another aspect, the invention provides a method of modifying a living cell to create an inducible and stable phenotypic alteration in the cell. For example, such a method comprising the step of transforming or transfecting a living cell with a polynucleotide according to the invention, wherein the polynucleotide includes a promoter sequence to promote expression of the encoded chimeric polypeptide in the cell, the promoter being inducible to promote increased expression of the chimeric polypeptide to a level that induces aggregation of the chimeric polypeptide into higher-ordered aggregates such as fibrils. In one preferred embodiment, the method further comprises the step of growing the cell under conditions which induce the promoter, thereby causing increased expression of the polypeptide and inducing aggregation of the chimeric polypeptide into aggregates or fibrils in the cell. In a highly preferred embodiment, the host cell lacks any native protein that contains the same SCHAG amino acid sequence that might co-aggregate with the chimeric polypeptide. For example, the SCHAG amino acid sequence comprises an amino terminal domain of a Sup35 protein, and the host cell is a yeast cell that comprises a mutant Sup35 gene that expresses a Sup35 protein lacking an amino terminal domain capable of prion aggregation. In such host cells, the chimeric polypeptide can be expressed at a high level and induced to aggregate without concomitant precipitation of the host cell's Sup35 protein into the aggregates, which could be detrimental to host cell viability.

In yet another aspect, the invention provides methods for reverting the phenotype obtained according to the method described in the preceding paragraph. One such method comprises the step of overexpressing a chaperone protein in the cell to convert the polypeptide from a fibril-forming conformation into a soluble conformation. In a preferred embodiment, the chaperone protein comprises the Hsp104 protein of yeast, or a related Hsp100-type protein from another species. Examples include the ClpB protein of E. coli and the At101 protein of Arabidopsis. [See generally Schirmer et al., Trends in Biochemistry, 21: 289-296 (1996), incorporated herein by reference.] The over-expression is achieved, e.g., by placing the gene encoding the chaperone protein under the control of an inducible promoter and inducing the promoter.

Another such method for reverting the phenotype comprises the step of contacting the cell with a chemical denaturant at a concentration effective to convert the polypeptide from a fibril-forming conformation to a soluble conformation. Exemplary denaturants include guanidine HCl (preferably about 0.1 to 100 mM, more preferably 1-10 mM) and urea. In another variation, the cell is subjected to heat or osmotic shock for a period of time effective to convert the polypeptide's conformation. Both over-expression of Hsp104 and growth on guanidine-HCl containing medium have proven effective for inducing phenotypic reversion of chimeric NM-GR prion constructs described in the Examples herein.

In yet another aspect, the invention provides materials and methods for identifying novel SCHAG amino acid sequences. One such method comprises the steps of joining a candidate nucleotide sequence “X” to a nucleotide sequence encoding the carboxyl terminal domain of a Sup35 protein (CSup35), especially a yeast Sup35 protein, to create a chimeric polynucleotide of the formula 5′-XCSup35-3′ or 5′-CSup35X-3′; transforming or transfecting a host cell with the chimeric polynucleotide; growing the host cell under conditions in which the host cell loses its native Sup35 gene, such that the chimeric polynucleotide becomes the only polynucleotide encoding CSup35; growing the resultant host cell under conditions selective for a nonsense suppressive phenotype; and selecting a host cell displaying the nonsense suppressive phenotype, wherein growth in the selective conditions is correlated with the candidate nucleotide sequence X encoding a SCHAG amino acid sequence. Additional methods steps and alternative methods are. described in detail below in the Examples. In one variation, the Csup35 is substituted by a different protein domain for which selection on the basis of inactivation is possible.

Many of the foregoing aspects of the invention relate, at least in part, to embodiments that involve chimeric polynucleotides and polypeptides, wherein properties of SCHAG amino acid sequences are advantageously employed through attaching them to other sequences using recombinant molecular biological techniques. In another variation of the invention, the advantageous properties of SCHAG amino acid sequences are exploited by making SCHAG sequences with sites that are modifiable using organic chemistry or enzymatic techniques.

For example, in one embodiment, the invention provides a method of making a reactable SCHAG amino acid sequence comprising the steps of identifying a SCHAG amino acid sequence, wherein polypeptides comprising the SCHAG amino acid sequence are capable of forming ordered aggregates; analyzing the SCHAG amino acid sequence to identify at least one amino acid residue in the sequence having a side chain exposed to the environment in an ordered aggregate of polypeptides that comprise the SCHAG amino acid sequence; and modifying the SCHAG amino acid sequence by substituting an amino acid containing a reactive side chain for the amino acid identified as having a side chain exposed to the environment in an ordered aggregate of polypeptides that comprise the SCHAG amino acid sequence. By “reactive” side chain is meant an amino acid with a charged or polar side chain that can be used as a target for chemical modification using conventional organic chemistry procedures, preferably procedures that can be performed in an environment that will not permanently denature the protein. In preferred embodiments, the amino acid containing a reactive side chain is cysteine, lysine, tyrosine, glutamate, aspartate, and arginine. The identifying step entails any selection of a SCHAG amino acid sequence. For example, the identifying can simply entail selecting one of the SCHAG amino acid sequences described in detail herein; or can entail screening of genomes, proteins, or phenotypes of organisms to identify SCHAG sequences (e.g., using methodologies described herein); or can entail de novo design of SCHAG sequences based on the properties described herein.

Proteins comprising the SCHAG sequence are capable of coalescing into higher-ordered aggregates. The polypeptides of such aggregates have amino acids that are disposed internally (in close proximity only to other amino acids in the aggregate), and other amino acids whose side chains are exposed to the environment of the aggregate such that they contact molecules in the environment. In the method, the analyzing step entails a prediction or a determination of at least one amino acid within the SCHAG sequence that is exposed to the environment of an aggregate of the proteins, meaning that it is an amino acid that will likely contact chemical reagents that mixed with the aggregates.

Amino acids in a SCHAG amino acid sequence having side chains exposed to the environment in ordered aggregates of polypeptides comprising the SCHAG amino acid sequence can be identified experimentally, for example, by structural analysis of mutants constructed using site-directed mutagenesis, e.g., high throughput cysteine scanning mutagenesis, as described in detail below in the Examples. Alternatively, specific amino acids in a SCHAG amino acid sequence can be predicted to have side chains that are exposed to the environment in ordered aggregates of polypeptides comprising the SCHAG amino acid sequence based on structural studies or computer modeling of the SCHAG amino acid sequence. The step of modifying the amino acid sequence entails changing the identity of an amino acid within the sequence. For the purposes of such a method, the act of inserting a reactive amino acid within the amino acid sequence, at a position essentially adjacent to the position of the identified amino acid, is considered the equivalent of substituting that amino acid for the identified amino acid. In other words, for the purposes of making a reactable SCHAG amino acid sequence, the term “substituting” should be understood to include inserting an amino acid. within the amino acid sequence, at a position essentially adjacent to the position of the identified amino acid.

It is contemplated that some naturally-occurring SCHAG amino acid sequences will fortuitously include one or more reactive amino acids whose side chains are exposed to the environment in polypeptide aggregates. Use of such naturally occurring SCHAG reactive amino acids is contemplated as an additional aspect of the invention. Moreover, modification of naturally occurring SCHAG amino acid sequences that contain an undesirable number of reactive amino acids to eliminate one or more reactive amino acids is contemplated.

In a preferred embodiment, the method further comprises a step of making a polypeptide comprising the reactable SCHAG amino acid sequence. Substitution of such amino acids with amino acid residues containing reactive side chains can be carried out in the laboratory by, e.g., site-directed mutagenesis of a SCHAG-encoding polynucleotide or by peptide synthesis of the SCHAG amino acid sequence. In another preferred embodiment, the invention additionally comprises the step of making a polymer comprising an ordered aggregate of polypeptide monomers wherein at least one of the polypeptide monomers comprises a reactable SCHAG amino acid sequence. For example, polypeptide monomers comprising the reactable SCHAG amino acid sequence are seeded with an aggregate or, otherwise subjected to an environment favorable to the formation of an ordered aggregate or “polymer” of the polypeptide monomers. In yet another preferred embodiment, the invention further comprises the step of contacting the reactive side chains with a chemical agent to attach a substituent to the reactive side chains. The substituent itself may be a linker molecule to facilitate attachment of one or more additional molecules. The substituent may be attached using a chemical agent. Attachment of a substituent depends on the nature of the substituent, as well as the identity of the reactive side chain, and can be accomplished by conventional organic chemistry procedures. Exemplary procedures for modifying the sulfhydryl group of a cysteine residue that has been introduced into a SCHAG amino acid sequence are described in greater detail below in the Examples. In preferred embodiments, the substituent is an enzyme, a metal atom, an affinity binding molecule having a specific affinity binding partner, a carbohydrate, a fluorescent dye, a chromatic dye, an antibody, a growth factor, a hormone, a cell adhesion molecule, a toxin, a detoxicant, a catalyst, or a light-harvesting or light altering substituent. In a preferred embodiment, the reactive amino acid that has been introduced into the SCHAG sequence will be substantially absent from the rest or the SCHAG amino acid sequence, or at least substantially absent from those portions of the sequence that are exposed to the environment in ordered aggregates of the polypeptide. This absence may be a natural feature, or may be the result of an additional modification step to substitute or delete other occurrences of the amino acid. Designing the reactable SCHAG amino acid sequence in this manner permits controlled chemical modification at the reactive sites that have been designed into the sequence, without modification of other residues.

In yet another embodiment of the invention, the invention further comprises the steps of contacting the polypeptides comprising the reactive side chains with a chemical agent to attach a substitutent to the reactive side chains, thereby providing modified polypeptides, and making a polymer comprising an ordered aggregate of polypeptide monomers, wherein at least some of the polypeptide monomers comprise the modified polypeptides. Exemplary procedures for making a polymer comprising an ordered aggregate of modified polypeptide monomers are described in greater detail below in the Examples.

In yet another embodiment, the invention provides a method of making a reactable SCHAG amino acid sequence, wherein the SCHAG amino acid sequence is modified to contain exactly one, two, three, four, or some other specifically desired number of the reactive amino acids. An exemplary method comprises the steps of (a) identifying a SCHAG amino acid sequence, wherein polypeptides comprising the SCHAG amino acid sequence are capable of forming ordered aggregates; (b) analyzing the SCHAG amino acid sequence to identify at least one amino acid residue in the sequence having a side chain exposed to the environment in an ordered aggregate of polypeptides that comprise the SCHAG amino acid sequence; (c) modifying the SCHAG amino acid sequence by substituting an amino acid containing a reactive side chain for the amino acid identified as having a side chain exposed to the environment in an ordered aggregate of polypeptides that comprise the SCHAG amino acid sequence; (d) analyzing the SCHAG amino acid sequence to identify at least a second amino acid residue in the sequence having an amino acid side chain that is exposed to the environment in an ordered aggregate of polypeptides that comprise the SCHAG amino acid sequence; and (e) modifying the SCHAG amino acid sequence by substituting an amino acid containing a reactive side chain for at least one amino acid identified according to step (d), wherein the amino acid substituted in steps (c) and (d) differ, thereby making a reactable SCHAG amino acid sequence with at least two selectively reactable sites. This method can be further elaborated to create SCHAG amino acids sequences with more than two selectively reactable sites. By introducing two or more different reactive amino acids, a SCHAG sequence is created with two or more sites that can be separately reacted/modified. It will be appreciated that the method also can be performed to introduce the same reactive amino acid for each identified amino acid, to create two or more identical reactive sites in the SCHAG sequence.

In another embodiment of the invention, the invention provides polypeptides comprising a SCHAG amino acid sequence that has been modified by substituting at least one amino acid that is exposed to the environment in an ordered aggregate of the polypeptides with an amino acid containing a reactive side chain, as well as polynucleotides that encode the polypeptides. In a further embodiment, a substituent is attached to the reactive amino acid of the modified polypeptide of the invention or reactable SCHAG sequence. In a highly preferred embodiment, the SCHAG amino acid sequence is modified to contain exactly one, two, three, four, or some other specifically desired number of the reactive amino acids, thereby providing a SCHAG amino acid sequence which is modifiable at controlled, stoichiometric levels and positions. To achieve this goal, modifications to remove undesirable, native reactive amino acids from a naturally occurring SCHAG sequence are contemplated. Polypeptides comprising a naturally occurring SCHAG amino acid sequence characterized by one or more reactive amino acids, that have been modified by substituting or eliminating a natural reactive amino acid, are considered a further aspect of the invention, as are polynucleotides that encode the polypeptides.

In still another variation, the invention provides various living cells with two or more customized, reversible phenotypes. For example, the invention provides a living cell that comprises: (a) a first polynucleotide comprising a nucleotide sequence encoding a polypeptide that comprises a prion aggregation domain and a domain having transcription or translation modulating activity, wherein the living cell is capable of existing in a first stable phenotypic state characterized by the polypeptide existing in an unaggregated state and exerting a transcription or translation modulating activity and a second phenotypic state characterized by the polypeptide existing in an aggregated state and exerting altered transcription or translation modulating activity; and (b) an exogenous polynucleotide comprising a nucleotide sequence that encodes a polypeptide of interest, with the proviso that the sequence encoding the polypeptide of interest includes a regulatory sequence causing differential expression of the polypeptide in the first phenotypic state compared to the second phenotypic state. Exemplary prion aggregation domains are described with respect to Sup35, Rnq1, and Ure2. The first polynucleotide may itself be an endogenous (native) polynucleotide of the cell, such as the native yeast Sup35 sequence in a yeast cell, which comprises a prion aggregation domain fused to a translation termination factor sequence. Alternatively, the first polynucleotide may be introduced into the cell (or a parent cell) using genetic engineering techniques. The term “exogenous polynucleotide” is meant to encompass any polynucleotide sequence that differs from a naturally occurring sequence in the cell as a result of human genetic manipulation. For example, an exogenous sequence may constitute an expression construct that has been introduced into a cell, such as a construct that contains a promoter, a foreign polypeptide-encoding sequence, a stop codon, and a polyadenylation signal sequence. Alternatively, an exogenous sequence may constitute an endogenous polypeptide-encoding sequence that has been modified only by the introduction of a promoter, an enhancer, or other regulatory sequence that is not naturally associated with the polypeptide-encoding sequence. Introduction of a regulatory sequence that is influenced by the aggregation state of the polypeptide encoded by the first polynucleotide is specifically contemplated. In one preferred variation, the cell further comprises a nucleotide sequence that encodes a polypeptide that modulates the expression level or conformational state of the polypeptide that comprises the prion aggregation domain. Such a polynucleotide facilitates manipulation of the cell to switch phenotypes. Polynucleotides encoding chaperone proteins that influence prion protein folding represent one example of this latter category of polynucleotide. In one specific variation, the invention provides a living cell according to claim 97, wherein the first polynucleotide comprises a nucleotide sequence encoding a polypeptide that comprises a prion aggregation domain fused in-frame to a nucleotide sequence encoding a translation termination factor polypeptide; and wherein the regulatory sequence comprises a stop codon that interrupts translation of the polypeptide of interest.

In another variation, the invention provides a living cell comprising: (a) a polynucleotide comprising a nucleotide sequence encoding a polypeptide that comprises a prion aggregation domain fused in-frame to a nucleotide sequence encoding a translation termination factor polypeptide; and (b) an exogenous polynucleotide comprising a nucleotide sequence that encodes a polypeptide of interest, with the proviso that the sequence encoding the polypeptide of interest includes at least one stop codon that interrupts translation of the polypeptide of interest; wherein the living cell is capable of existing in a first stable phenotypic state characterized by translational fidelity and substantial absence of synthesis of the polypeptide of interest and a second phenotypic state characterized by aggregation of the translation termination factor, reduced translational fidelity, and expression of the polypeptide of interest.

The invention also provides polymers or fibers of ordered aggregates comprising polypeptide subunits wherein at least one of the polypeptide subunits comprises a reactable SCHAG amino acid sequence. By the term “fibril” or “fiber” is meant a filamentous structure composed of higher ordered aggregates. By “polymer” is meant a highly ordered aggregate that may or may not be filamentous. In another embodiment, the polymer or fiber is modified or substituted by attaching a substituent to the reactable SCHAG amino acid sequence of the polypeptide subunits. Also contemplated are polymers or fibers that comprise more than one type of substituent by attachment of different substituents to the reactable SCHAG amino acid sequence of the polypeptide subunits of the polymer or fiber. Attachment of the substituents to the reactive side chains contained in the reactable SCHAG amino acid sequence can occur either before or after coalescing of the polypeptides comprising the reactable SCHAG amino acid sequences into polymers comprising ordered aggregates of the polypeptides. Modification by attachment of specific substituents to such polymers or fibers can confer distinct functions to these molecules. Thus, polymers or fibers, wherein one or more discrete regions of the polymer or fiber are modified to enable a distinct function are contemplated. In another variation, different regions of a polymer or fiber are differentially modified to confer different functions. Also contemplated are polymers or fibers containing patterns of attachments, and consequently patterns of functionalities. The invention also provides polymers comprising fibers wherein at least one fiber has a distinct function different from that of another fiber in the polymer. Fibers comprising polypeptides subunits that are capable of emitting light or altering the wavelength of the light emitted in response to binding of a ligand to the fiber can be used as highly sensitive biosensors. Polymers comprising fibers wherein some of the fibers comprise polypeptide subunits capable of absorbing light of one wavelength and emitting light of second wavelength, and other fibers comprising polypeptide subunits capable of absorbing the light emitted by the first set of fibers and emitting light of a different wavelength are also contemplated.

In one preferred embodiment, the polymer or fiber is long and thin and contains no or few branches, except at positions defined by deliberate introduction of sites for interaction between the polypeptide subunits. Polymers or fibers in which the polypeptide subunits have been modified to enable directed interactions between the polypeptide subunits within a single polymer or fiber, or between two discrete polymers or fibers are contemplated. Polymers of fibers that have been modified to enable interactions to occur between separate polymers of fibers can be used to create a meshwork of polymers of fibers. In one variation, the meshwork can be generated reversibly by using interactions dependent on sulfhydryl groups present on the polypeptide subunits of the polymer of fiber. Such meshworks can be useful, for example, for filtration purposes. In another preferred embodiment, a fibril, ordered aggregate, polymer or fiber is attached to a solid support. For example, binding of a polymer of fiber to a solid support can be mediated by biotin-avidin interactions, wherein the biotin is attached to the polymers or fibers and avidin is bound to the solid support or vice versa.

In a related embodiment, the invention provides a method of making a polymer or fiber with a predetermined quantity of reactive sites for chemically modifying the polymer of fiber, comprising the steps of providing a first polypeptide comprising a first SCHAG amino acid sequence that is capable of forming ordered aggregates with polypeptides identical to the first polypeptide; providing a second polypeptide comprising a second SCHAG amino acid sequence that is capable of forming ordered aggregates with polypeptides identical to the first polypeptide or the second polypeptide, wherein the second SCHAG amino acid sequence includes at least one amino acid residue having a reactive amino acid side chain that is exposed to the environment and serves as a reactive site in ordered aggregates of the second polypeptide and; mixing the first and second polypeptides under conditions favorable to aggregation of the polypeptides into ordered aggregates, wherein the polypeptides are mixed in quantities or ratios selected to provide a predetermined quantity of second polypeptide reactive sites. In a preferred embodiment, the invention further comprises the step of reacting the reactive side chains to attach a substituent to the reactive amino acid side chains of the polymer of fiber. Alternatively, the step of reacting the reactive side chains to attach a substituent to the reactive amino acid side chains is performed prior to mixing of the polypeptides comprising reactable SCHAG amino acid sequences to from ordered aggregates. In yet another embodiment, the invention provides a method of making a polymer or fiber comprising a first polypeptide comprising a first SCHAG amino acid sequence and a second polypeptide comprising a second SCHAG amino acid sequence, wherein both the first and second SCHAG amino acid sequence includes at least one amino acid residue having a reactive amino acid side chain that is exposed to the environment and serves as a reactive site, and wherein the reactive amino acid side chains of the first and second SCHAG amino acid sequences that are exposed to the environment in ordered aggregates are not identical, thereby permitting selective reaction of the reactive amino acid side chain of the first SCHAG amino acid sequence without reacting the reactive amino acid side chain of the second SCHAG amino acid sequence.

In another embodiment, the invention provides a method of making a polymer comprising two or more regions with distinct function comprising the steps of (a) providing a first polypeptide comprising a SCHAG amino acid sequence and a first functional domain and a second polypeptide comprising a SCHAG amino acid domain and a second functional domain that differs from the first functional domain, wherein the SCHAG amino acid sequences of the polypeptides are capable of forming ordered aggregates with polypeptides identical to the first or second polypeptide; (b) aggregating the first polypeptide by subjecting a composition comprising the first polypeptide to conditions favorable to aggregation of the first polypeptide into ordered aggregates, thereby forming a polymer comprising a region containing polypeptides that include the first functional domain; and (c) mixing a composition comprising the second polypeptide with the polymer formed according to step (b), under conditions favorable to aggregation of the second polypeptide with the polymer of step (b), thereby forming a polymer comprising the first region containing polypeptides that include the first functional domain and a second region containing polypeptides that include the second functional domain.

In one preferred embodiment, the SCHAG amino acid sequences of the first and second polypeptides are identical. In another preferred embodiment, at least one of the first and second functional domains comprises an amino acid that comprises a reactive amino acid side chain. In yet another preferred embodiment, at least one of the first and second functional domains comprises an amino acid sequence of a polypeptide of interest. In another variation, the method further comprises the step of mixing a composition comprising the first polypeptide with the polymer formed according to step (c), under conditions favorable to aggregation of the first polypeptide with the polymer of step (c), thereby forming a polymer comprising the first region containing polypeptides that include the first functional domain, the second region containing polypeptides that include the second functional domain, and a third region containing polypeptides that include the first functional domain. Alternatively, the invention provides a method of making a polymer comprising two or more regions with distinct function wherein the method further comprises the steps of providing a third polypeptide that comprises a SCHAG amino acid sequence and a third functional domain that differs from the first and second functional domains, wherein the SCHAG amino acid sequence of the third polypeptide is capable of forming ordered aggregates with polypeptides identical to the first polypeptide or the second polypeptide; and mixing a composition comprising the third polypeptide with the polymer formed according to step (c), under conditions favorable to aggregation of the third polypeptide with the polymer of step (c), thereby forming a polymer comprising the first region containing polypeptides that include the first functional domain, the second region containing polypeptides that include the second functional domain, and a third region containing polypeptides that include the third functional domain.

Additional features and variations of the invention will be apparent to those skilled in the art from the entirety of this application, including the drawing and detailed description, and all such features are intended as aspects of the invention. Likewise, features of the invention described herein can be re-combined into additional embodiments that also are intended as aspects of the invention, irrespective of whether the combination of features is specifically mentioned above as an aspect or embodiment of the invention. Also, only such limitations which are described herein as critical to the invention should be viewed as such; variations of the invention lacking limitations which have not been described herein as critical are intended as aspects of the invention.

In addition to the foregoing, the invention includes, as an additional aspect, all embodiments of the invention narrower in scope in any way than the variations specifically mentioned above. Although the applicant(s) invented the full scope of the claims appended hereto, the claims appended hereto are not intended to encompass within their scope the prior art work of others. Therefore, in the event that statutory prior art within the scope of a claim is brought to the attention of the applicants by a Patent Office or other entity or individual, the applicant(s) reserve the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention.

### BRIEF DESCRIPTION OF THE DRAWING

FIG. 1 depicts the DNA and deduced amino acid sequences (SEQ ID NOs: 50-51) of an NMSup35-GR chimeric gene described in Example 1.

FIG. 2 depicts a map of an integration plasmid described in Example 2 which contains a chimeric gene comprising the amino-terminal domain of yeast Ure2 protein, a hemagglutinin tag sequence, and the carboxyl-terminal domain of yeast Sup35 protein.

FIG. 3 depicts the nucleotide sequence (SEQ ID NO: 49) of the plasmid of FIG. 2. As shown in FIG. 2, the NUre2-CSup35 chimeric gene is encoded on the strand complementary to the strand whose sequence is depicted in FIG. 3.

FIG. 4 schematically depicts that the structure of wild-type (WT) yeast Sup35 protein (Top), which contains an amino-terminal region characterized by five imperfect short repeats, a highly charged middle (M) region, and a carboxyl-terminal region involved in translation termination during protein synthesis; a Sup35 mutant designated RΔ2-5, characterized by deletion of four of the repeat sequences in the N region; and a Sup35 mutant designated R2E2 (bottom), into which two additional copies of the second repeat segment have been engineered into the N region. Also depicted is the frequency with which yeast strains carrying these various Sup35 constructs were observed to spontaneously convert from a [psi−] to a [PSI⁺] phenotype.

FIG. 5 depicts gold and silver enhancement of NM fibers. Long NM^{K184C} fibrils were assembled by seeding soluble NM^{K184C} with short NM^{K184C} fibrils. Monomaleimido Nanogold was covalently cross-linked (2) and the 1.4-nm Nanogold particles were subjected to gold toning (3-4). Fibrils are labeled as 1; nanogold particles are labeled as 2; silver particles are labeled as 3; and gold particles are labeled as 4.

FIG. 6 depicts gold toning is specific to labeled fibers. The resulting gold-toned fibers show a significant increase in height from 9-11 nm (bare fibers, labeled as 1) to 80-200 nm (labeled fibers, labeled as 2), imaged by AFM.

FIG. 7 depicts gold nanowires that did not bridge the gap when randomly deposited on patterned electrodes and imaged by TEM.

FIG. 8 shows depicts gold nanowires bridging the gap between two electrodes.

FIG. 9 depicts vaporization of some conducting nanowires after increasing the voltage. Conductive nanowires are labeled as 1, while vaporized nanowires are labeled as 2.

FIG. 10 schematically depicts an electrical circuit. A power source (i.e., electrical source) is labeled as 1; electrical conductors are labeled as 2; and circuit elements are labeled as 3.

### DETAILED DESCRIPTION OF THE INVENTION

The invention described herein is related to the invention described in U.S. patent application Ser. No. 09/591,632, filed Jun. 9, 2000, which claims priority benefit of U.S. Provisional Application No. 60/138,833, filed Jun. 9, 1999. Both of these applications are incorporated herein by reference.

The present invention expands the study of prion biology beyond the contexts where it has heretofore focused, namely fundamental research directed to developing a greater understanding of prion biology and medical research directed to developing diagnostic and therapeutic materials and methods for prion-associated disease states, and provides diverse and practical applications that advantageously employ certain unique properties of prions, including one or more of the following:
(1) prion genes and proteins afford the possibility of two stable, heritable phenotypes and the ability to effect at least one switch between such phenotypes;
(2) prions provide the ability to sequester a protein or protein-binding molecule into an ordered aggregate;
(3) prion protein aggregates are easily isolated from cells containing them; with at least some prions, the ordered aggregate is fibrillar in structure, stable and unreactive, a collection of properties that is exploited in certain embodiments of the invention;
(4) a protein of interest that is fused to a prion protein can potentially retain its normal biological activity even when the fusion has formed an ordered prion aggregate;
(5) a protein of interest that is fused to a prion protein can switch from an active to an inactive state, and this change is reversible;
(6) prion protein aggregates form fibrils with unusually high chemical and thermal stability for biological material; and.
(7) prion protein aggregates form fibrils that can be modified to incorporate specific functionalities, thereby combining the advantages of biomolecules with, for example, electronic circuitry.

Prion proteins have been observed to exist in at least two stable conformations in cells that synthesize them. For example, the PrP protein in mammals has been observed in a soluble PrP^{C} conformation in “normal” cells and in an aggregated, insoluble PrP^{Sc} conformation in animals afflicted with transmissible spongiform encephalopathies. Similarly, the Sup35 protein in yeast has been observed in a “normal” non-aggregated conformation in which it forms a component of a translation termination factor, and also aggregated into fibril structures in [PSI⁺] yeast cells (characterized by suppression of normal translation termination activity). To the extent that scientific literature has ascribed any practical importance to these observations, the importance has focused on identifying materials and methods to modulate conformational switching, which might lead to treatments for prion-mediated diseases; or to detect the infectious PrP^{Sc} form to protect the food supply; or to diagnose infection and prevent its spread. At least in the case of the yeast Sup35 prion, the [PSI⁺] phenotype can be eliminated by effecting an over-expression or under-expression of the heat shock protein Hsp104, and can be induced by effecting an over-expression of Sup35 or the Sup35 amino-terminal prion-aggregation domain.

The practical applications that arise from the ability to alter the phenotype of a cells or an entire organism by transforming/transfecting cells with a polynucleotide that encodes a non-native protein (and/or that integrates into the cell's genome to cause production of a non-native protein) are legion and underlie a major portion of the entire biotechnology industry. Such applications include medical/therapeutic applications (e.g., gene therapy to treat genetic disorders such as hemophilia; gene therapy to treat pathological conditions such as ischemia, inborn errors of metabolism, restenosis, or cancer); pharmacological applications (e.g., recombinant production of therapeutic polypeptides such as erythropoietin, human growth hormone, angiogenic and anti-angiogenic peptides, or cytokines for therapeutic administration); industrial applications (e.g., genetic engineering of microorganisms for bioremediation or frost prevention; or recombinant production of catalytic enzymes, vitamins, proteins, or other organic molecules for use in chemical and food processing); and agricultural applications (e.g., genetic engineering of plants and livestock to promote disease resistance, faster growth, better nutritional value, environmental durability, and other desirable properties); just to name a few. In such biotechnology applications, a cell typically is transformed/transfected with a single novel gene to introduce a single phenotypic alteration that persists as long as the gene is present. Means of controlling the new phenotype conventionally involve eliminating the new gene, or possibly placing the gene under the control of inducible or repressible promoter to control the level of gene expression. The present invention provides the realization that prion genes and proteins afford an additional, alternative means of biological control, because the introduction of a prion sequence into a protein introduces the possibility of two stable, heritable phenotypes and the ability to effect at least one switch between such phenotypes. Specifically, one can phenotypically alter a cell to produce a protein of interest by transforming/transfecting a cell with a gene encoding a prion-aggregation domain fused to a protein of interest. To reduce or eliminate the activity of this protein, one induces the protein to undergo a conformational alteration and adopt a prion-like aggregating phenotype, thereby sequestering the protein. To re-introduce the original recombinant phenotype, one induces the protein to undergo a conformational alteration and adopt the soluble phenotype.

By way of example, the phenotypic alteration potential of prion-like proteins can be harnessed to permit a species (plant, animal, microorganisms, fungi, etc.) to survive in a wider range of environmental conditions and/or quickly adopt to environmental changes. Species that thrive in one environment often have difficulty in another. For example, some photosynthetic organisms grow well under bright light because they produce pigments that protect the organism from potentially toxic effects of bright light, whereas others grow well under low light conditions because of other light-gathering pigment systems that efficiently harvest all available light. By placing the regulators for such systems under a prion control mechanism, prion conformational switching is advantageously harnessed for increased enviromnental adaptability.

A preferred prion system for harnessing environmental adaptation is a prion system such as the Sup35 or Ure2 yeast prions that undergo natural switching. In these systems, the yeast prion state and phenotype arises naturally (in a non-prion population) at a frequency of about one per million cells, and is lost at a similar frequency in a prion population. Thus, in any yeast culture of reasonable size, both phenotypes will be present. If the prion state imparts a growth advantage under some conditions and the non-prion state imparts a growth advantage under other conditions, the culture as a whole will survive and thrive under either set of conditions. Although one phenotype may be disfavored and selected against, it will nonetheless be present (due to natural switching behavior of the prion) and ready to “take over” the culture if conditions change to favor it. In this regard, also contemplated as an aspect of the invention is a cell culture comprising cells transformed or transfected with a polynucleotide according to the invention, wherein the cells express the chimeric polypeptide encoded by the polynucleotide, and wherein the cell culture includes cells wherein the chimeric polypeptide is present in an aggregated state and cells free of aggregated chimeric polypeptide.

The prion-mediated flexibility described in the preceding paragraph possesses a crucial advantage over traditional “switches” because it does not depend upon fortuitous genetic mutations and reversions. Each phenotype arises from the same genotype and each is available within the population, even under selective conditions. Thus, in a cultured photosynthetic organism as described above, transformation with one or more genes encoding an aggregating domain fused to pigment or protective proteins will provide an increased adaptability to varying light conditions.

This “natural switching” quality of prions has applicability to a wide variety of variable growth conditions that might be encountered by cultured cells or organisms, including varied levels of salinity, metals, carbon sources, and toxic metabolic byproducts. Adaptability to such environments is often mediated by one or a few proteins, such as metal-binding proteins and enzymes involved in the synthesis or breakdown of particular organic compounds. The advantages of prion natural switching are considered particularly well suited for fields of bioremediation, where multiple environmental conditions are expected to be encountered, and fermentation processes where nutrients are consumed and fermentation by products are created, changing an environment over time.

By way of another example, pigment genes for flowers, textile fibers (e.g., cotton), or animal fibers (e.g., wool) are placed under the control of prion-like aggregating elements. A plurality of colors and/or color patterns is achieved in a single plant by altering growing conditions to induce or cure the prion regulated pigment, or by subjecting portions of the plant to chemical agents that modulate conformation of the prion protein.

The present invention also provides practical applications stemming from the realization that prions provide the ability to sequester a protein of interest or the protein's binding partner into an ordered aggregate. This property is demonstrated herein by way of example involving the prion aggregation domain of the yeast Sup35 gene fused to a glucocorticoid receptor. When cells expressing this fusion are in a non-prion phenotype (i.e., the fusion protein is soluble), the cells are susceptible to hormonal induction through the glucocorticoid receptor, and one can induce the expression of a second gene that is operably fused to a glucocorticoid response element. However, when cells expressing the fusion are in a prion phenotype (i.e., the fusion protein is forming aggregates), the susceptibility to hormonal induction is reduced, because the glucocorticoid receptor that is sequestered into cytoplasmic aggregates is unable to effect its normal activity in the cell's nucleus.

This ability to a sequester protein or protein-binding partner has direct application in the recombinant production of biological molecules, especially where recombinant production is difficult using conventional techniques, e.g., because the molecule of interest appears to exert a toxic or growth-altering effect on the recombinant host cell. Such effects can be reduced, and production of the polypeptide of interest enhanced, by expressing the polypeptide of interest as fusion with a prion aggregation domain in a host cell that has, or is induced to have, a prion aggregation phenotype. In such host cells, the recombinant fusion protein forms ordered aggregates through its prion aggregation domain, thereby sequestering the protein of interest as part of the aggregate, and reducing its adverse effects on other cellular components or reactions. (If the molecule of interest is the binding partner of the non-prion domain of the fusion protein, the binding partner also will be sequestered by the aggregate, provided that the binding activity of this domain is retained in the aggregate.)

The present inventors also provide practical applications stemming from the fact that prion aggregates can be readily isolated from cells containing them. Because prions form insoluble aggregates in appropriate host cells, it is relatively easy to separate aggregated prion protein from most other proteinaceous and non-proteinaceous matter of a host cell, which is comparatively more soluble, using centrifugation techniques. When the prion protein is fused to a protein of interest, the protein of interest can likewise be separated from most other host cell impurities by centrifugation techniques. Thus, the present invention provides materials and methods useful for the purification of virtually any recombinant protein of interest. If a recognition sequence for chemical or enzymatic cleavage is included between the prion aggregation domain and the protein of interest, the protein of interest can be cleaved and separated from the insoluble prion aggregate in a second purification step. Such protein production techniques are considered an aspect of the invention. For example, the invention provides a method comprising the steps of: expressing a chimeric gene in a host cell, the chimeric gene comprising a nucleotide sequence encoding a SCHAG amino acid sequence fused in frame to a nucleotide sequence encoding a protein of interest; subjecting the host cell, or a lysate thereof, or a growth medium thereof to conditions wherein the chimeric protein encoded by the chimeric gene aggregates; and isolating the aggregates. In one variation, the method further includes the step of cleaving the protein of interest from the SCHAG amino acid sequence and isolating the protein of interest.

Moreover, the improved purification techniques are not limited to proteins fused to a prion domain. For example, a host cell expressing a prion aggregation domain fused to a protein of interest can be used in a like manner to purify a binding partner of the protein of interest. For example, if the protein of interest is a growth factor receptor, it can be used to sequester the growth factor itself by virtue of the receptor's affinity for the growth factor. In this way, the growth factor can be similarly purified, even though it is not itself expressed as a prion fusion protein. If the protein of interest comprises an antigen binding domain of an antibody, then the same techniques can be used to sequester and purify virtually any antigen (protein or non-protein) that is produced by the host cell or introduced into the host cell's environment. In this regard, it is well-known in the literature that relatively short variable (V) regions within antibodies are largely responsible for highly specific antigen-antibody immunoreactivity, and such antigen-binding regions occur within particular regions of an antibody's primary structure and are susceptible to isolation and cloning. (See, e.g., Morrison and Oi, Adv. Immunol., 44:65-92 (1989). For example, the variable domains of antibodies may be cloned from the genomic DNA of a B-cell hybridoma or from cDNA generated from mRNA isolated from a hybridoma of interest. Likewise, it is known in the art how to isolate only those portions of the variable region gene fragments that encode antigen-binding complementarity determining regions (“CDR”) of an antibody, and clone them into a different polypeptide backbone. [See, e.g., Jones et al., Nature, 321:522-525 (1986); Riechmann et al., Nature, 332:323-327 (1988); Verhoeyen et al., Science, 239:1534-36 (1988); and Tempest et al., Bio/Technology, 9:266-71 (1991).] A polypeptide comprising an antigen binding domain of an antibody of interest might comprise only one or more CDR regions from an antibody, or one or more V regions from an antibody, or might comprise entire V region fragments linked to constant domains from the same or a different antibody, or might comprise V regions that have been cloned into a larger, non-antibody polypeptide in a way that preserves their antigen binding characteristics, or might comprise antibody fragments containing V regions, and so on. Also, it is known in the art to select and isolate polypeptides comprising antigen binding domains of antibodies using techniques such as phage display that obviate the need to immunize animals and work with native antibodies at all.

The present invention also provides practical applications stemming from the fact that at least some proteins of interest will retain their normal biological activity when expressed as a fusion with a prion aggregation domain, even when thefusion protein forms prion-like aggregates. This feature of the invention is demonstrated by way of example below using the S. cerevisiae Sup35 prion aggregation domain fused to a green fluorescent protein (GFP). Even in [PSI⁺] cells or in other cells where aggregation of the fusion protein into fibrils has occurred, the GFP fluoresces green under blue light, indicating that the GFP portion of the fusion has retained a biologically active conformation.

When the example is repeated substituting a protein of interest for the GFP marker protein, ordered aggregates comprising a biologically active protein of interest are produced. In a preferred embodiment, the protein of interest is a protein that is capable of binding a composition of interest. For example, the protein of interest comprises an antigen binding domain of an antibody that specifically binds an antigen of interest; or it comprises a ligand binding domain of a receptor that binds a ligand of interest. Fibrils comprising such fusion proteins can be used as affinity matrices for purifying the composition of interest. Thus, aggregates of a chimeric protein comprising a SCHAG amino acid sequence fused to an amino acid sequence encoding a binding domain of a protein having a specific binding partner are intended as an aspect of the invention.

In another preferred embodiment, the polypeptide of interest is an enzyme, especially an enzyme considered to be of catalytic value in a chemical process. Fibrils comprising such fusion proteins can be used as a catalytic matrix for carrying out the chemical process. Thus, aggregates of a chimeric protein comprising a SCHAG amino acid sequence fused to an enzyme are intended as an aspect of the invention.

In another preferred embodiment, ordered aggregates are created comprising two or more enzymes, such as a first enzyme that catalyzes one step of a chemical process and a second enzyme that catalyzes a downstream step involving a “metabolic” product from the first enzymatic reaction. Such aggregates will generally increase the speed and/or efficiency of the chemical process due to the proximity of the first reaction products and the second catalyst enzyme. Aggregates comprising two or more proteins of interest can be produced in multiple ways, each of which is itself considered an aspect of the invention.

It may be advantageous to attach fibers to a solid support such as a bead (e.g., a Sepharose bead) or a surface to create a “chip” containing loci with biological or chemical function.

In one variation, each chimeric protein comprising an aggregation domain and a protein of interest is produced in a separate and distinct host cell system and recovered (purified and isolated). The proteins are either recovered in soluble form or are solubilized. (Complete purification is desirable but not essential for subsequent aggregation/polymerization.) Thereafter, a desired mixture of the two or more proteins is created and induced into polymerization, e.g., by “seeding” with a protein aggregate, by concentrating the mixture to increase molarity of the proteins, or by altering salinity, acidity, or other factors. The desired mixture may be 1:1 or may be at a ratio weighted in favor of one chimeric protein (e.g., weighted in favor of an enzyme that catalyzes a slower step in a chemical process). The different chimeric proteins co-polymerize with the seed and with each other because they comprise compatible aggregation (SCHAG) domains, and most preferably identical aggregation domains. In certain embodiments it may be desirable to include in the pre-aggregation mixture a polypeptide comprising the SCHAG domain only, without an attached enzyme, for the purpose of increasing the average space between individual enzyme molecules in the aggregate that is formed. The additional space may be desirable, for example, if the enzyme's substrate is a large molecule.

In another variation, the two distinct host cell systems are co-cultured, and the chimeric transgenes include signal peptides to induce the cells to secrete the chimeric proteins into the common culture medium. The proteins can be co-purified from the medium or induced to aggregate without prior purification.

In still another variation, the transgenes for two or more recombinant chimeric polypeptides are co-transfected into the same host cell, either on a single polynucleotide construct or multiple constructs. Such a host cell produces both recombinant polypeptides, which can be induced to polymerize in vivo in a prion phenotype host, or can be recovered in soluble form and induced to polymerize in vitro. The present invention also exploits the fact that at least certain prion proteins form aggregates that are fiber-like in shape; strong; and resistant to destruction by heat and many chemical environments. This collection of properties has tremendous industrial application that heretofore has not been exploited. Thus, in one embodiment, the invention provides polypeptides comprising SCHAG amino acid sequences which have been modified to comprise a discrete number of reactive sites at discrete locations. The polypeptides can be recombinantly produced and purified and aggregated into robust fibers resistant to destruction. The reactive sites permit modification of the polypeptides (or the fibers comprising the polypeptides) by attachment of virtually any chemical entity, such as pigments, light-gathering and light-emitting molecules for use as sensors, indicators, or energy harnessing and transduction; enzymes; metal atoms; organic and inorganic catalysts; and molecules possessing a selective binding affinity for other molecules. Electrical fields may be applied to fibers that are labeled with metal atoms, so that the fibers can be oriented in a specific direction. Because the fiber monomers are protein, conventional genetic engineering techniques can be used to introduce any number of desired reactive sites at precise locations, and the precise location of the reactive sites can be studied using conventional protein computer modeling as well as experimental techniques. Proteins and fibers of this type enjoy the utilities of the chimeric proteins described above (e.g., as chemical purification matrices, chemical reaction matrices, etc.) and additional utility due to the ability to bind a potentially infinite variety of non-protein molecules of interest to the reactive sites. The fibers can be grown or attached to solid supports to create devices comprising the fibers.

In another preferred embodiment, the polypeptides of the present invention are used for the construction of nanostructures. For example, the N-terminal and middle region (NM) of yeast Saccharomyces cerevisiae Sup35p (i.e., NM) forms self-assembling β-sheet-rich amyloid fibers that are suitably sized and shaped for nanocircuitry with diameters of 9-11 nm (Glover, J. R., et al., Cell, 89: 811-819 (1997)). The highly flexible structure of soluble NM rapidly converts to form amyloid fibers when it associates with preformed fibers that act as seeds for fiber formation (Serio, T. R., et al., Science, 289: 1317-1321 (2000); Scheibel, T. & Lindquist, S. L., Nat. Struct. Biol., 8:958-962 (2001); DePace, A. H. & Weissman, J. S., Nat. Struct. Biol., 9, 389-396 (2002)). The fibers grow by extension from either end (Scheibel, T., et al., Curr. Biol., 11: 366-369 (2001)), and this bidirectional formation is useful for forming varied fiber patterns: a valuable property for the production of circuitry.

NM has several advantageous properties for manufacturing. NM fibers have a higher than average chemical stability as demonstrated by its resistance to proteases and protein denaturants (Serio, T. R., et al., supra). Indeed, PrP, the mammalian prion counterpart of Sup35p, is infamous for its extraordinary resistance to destruction. (However, neither Sup35p nor NM are infectious to humans and therefore can be handled safely.) The stability of NM suggests that it can withstand diverse metallization procedures necessary for creating electric circuits in industrial settings. In addition, NM fibers do not form aggregates as readily as other amyloids. Furthermore, under some circumstances such as different surface treatments, methods of fiber deposition, and solutions in which they are suspended, NM fibers tend not to aggregate with each other. The solubility of NM in physiological buffers greatly facilitates handling before and during fiber formation (Scheibel, T., et al., Curr. Biol., 11: 366-369 (2001)).

Moreover, among the various DNA and protein fibers that have been described, NM fibers are unusual in that they are highly resistant to extended periods at high temperatures, exposure to high and low salt, strong denaturants, strong alkalis and acids, and 100% ethanol. These properties will allow them to withstand the harsh conditions in industrial processes. Depending on the conditions, NM fibers can nucleate spontaneously or self-assemble from preformed nuclei (Scheibel, T. & Lindquist, S. L., Nat. Struct. Biol., 8:958-962 (2001)), an advantageous property for the practical assembly of circuits on a large scale. Further, the ability to manipulate the fiber length as described herein increases flexibility in designing nanostructures.

Bidirectional growth from NM seeded fibers can be used to incorporate NM derivatives with different modifications, interspacing them along individual fibers, e.g., with and without exposed cysteines. As different substrates can be prepared to bind to cysteine and to native lysine, these alternative binding sites provide flexibility and diversity in the patterning and mixing of substrates covalently bound to the fiber. Genetic engineering can be used to fuse a wide array of protein domains to the C-terminus of NM during its initial in vivo synthesis in such a way that the domains are tethered laterally, external to the surface of assembled fibers. Thus they remain functional even when NM is in its fibrous form.

Because many enzymes can function when attached to protein fibers, it is possible to incorporate more complex reaction centers into NM nanocircuitry, thereby creating electronic circuits that can take advantage of biological capacities. Mechanisms such as the vaporization of NM fibers with high voltages could act as a fuse or a switch to permanently activate or inactivate specific reaction centers within the circuitry.

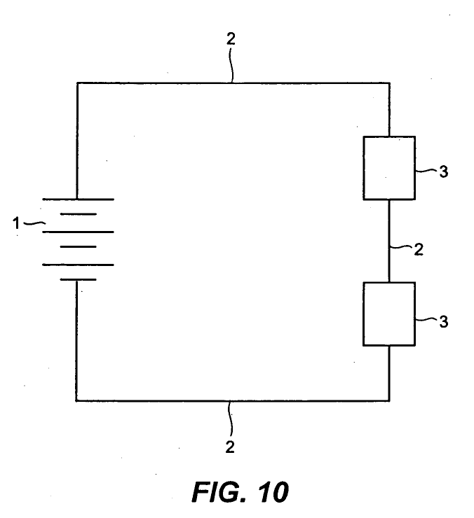

Fibril-based electrical conductors of the invention can be used as components in any product, device, or method of manufacture requiring electrical conductors. Due to their small size, electrical conductors of the invention are especially useful for small-scale devices such as microcircuits in nanodevices. Referring to FIG. 10, an exemplary circuit comprises a power source 1, one or more circuit elements 3, and electrical conductors (e.g., wires) disposed between the power source and the circuit elements 2 (and optionally between circuit elements). For example, a first location of the electrical conductor is attached to or contacts the power source and a second location of the electrical conduct is attached to or contacts a circuit element in a manner whereby the electrical conductor can conduct electricity between the power source and the circuit element (or between circuit elements). Circuit elements can be active or passive and can be any component that could be included in a circuit, such as a capacitor, an inductor, a resistor, an integrated circuit, an oscillator, a transistor, a diode, a switch, or a fuse.

There is a great opportunity to expand further the potential interconnections in these circuits by exploiting the natural diversity and strength of protein-protein interactions (Begley, T. J., et al., Mol. Cancer Res., 1: 103-112 (2002); Uetz, P., et al., Nature, 403: 623-627 (2000); Marcotte, E., et al., Nature, 402: 83-86 (1999)). Protein-protein interactions can be extremely specific and strong, as can the interactions of protein-ligand-protein. Such protein properties can be used as a mechanism to bring premetallized wires into juxtaposition in response to changes in physical conditions, the presence of ligands, and the appearance of partner proteins, etc. These connections are readily reversible (Schreiber, S. L. & Crabtree, G. R. Harvey Lect., 91: 99-114 (1995-1996); Spencer, D. M., et al., Science, 262: 1019-1024 (1993)).

Complex circuit schematics can be generated with NM fibers, initiated by patterned surface modifications (independently or in combination) such as lithography, growth in flows or magnetic field gradients, alignment by electrical fields, active patterning with optical tweezers, dielectrophoresis and 3D patterning using hydrogels or microfluidic channels (Korda, P., et al., Rev. Sci. Instrum. 73: 1956-1957 (2002); Kane, R. S., et al., Biomaterials 20: 2363-2376 (1999); Inouye, H., et al., Biophys. J. 64: 502-519 (1993); Luther, P. W., et al., Nature 303: 61-64 (1983); Kubista, M., et al., J. Biomol. Struct. Dyn. 8: 37-54 (1990); Hermanson, K. D., et al., Science 294; 1082-1086 (2001)). The feasibility of such maneuvers is demonstrated by the natural tendency of NM fibers to align with each other rather than to form dense intractable clumps characteristic of other protein amyloids and the conditions that produce such alignments can be optimized. Attachment of NM to patterned surfaces can be mediated via covalent bonds to native lysine residues, genetically engineered cysteine residues, or other novel residues or modifications.

The present invention provides a mechanism for generating robust nanowires that meet the needs of industrial processes with the potential to couple powerful combinations of biological processes and functionalities with electronic circuitry. In particular, these nanowires may be electrical conductors which may include any type of electrically conductive materials such as metal, like gold, silver, copper, etc., or semi-conductive materials such as known semi-conductors suited to conduct electricity either along the length of the nanowire, radially with respect to the nanowire, or a combination of both.

These and other aspects of the invention will be better understood by reference to the following examples. The examples are not intended to limit the scope of the invention, and variations will be apparent to the reader from the entirety of this document.

### EXAMPLE 1

### Construction and Assaying of a Chimeric, Prion-like Gene and Protein with Yeast Sup35 Protein

The following experiments were performed to demonstrate that a prion-determining domain of a prion-like protein can be fused to a polypeptide from a wholly different protein to construct a novel, chimeric gene and protein having prion-like properties. The relevance of these experiments to the present invention also is explained.

A. Construction of a NMSup35-GR Chimeric Gene

The yeast (Saccharomyces cerevisiae) Sup35 protein (SEQ ID NO: 2, 685 amino acids, Genbank Accession No. M21129) possesses the prion-like capacity to undergo a self-perpetuating conformational alteration that changes the functional state of Sup35 in a manner that creates a heritable change in phenotype. Experiments have demonstrated that it is the amino-terminal (N region, amino acids 1-123 of SEQ ID NO: 2) or the amino-terminal plus middle (M, amino acids 124-253 of SEQ ID NO: 2) regions of Sup35 that are responsible for this prion-like capacity. See Glover et al., Cell, 89: 811-819 (1997); see also King et al., Proc. Natl. Acad. Sci. USA, 94:6618-6622 (1997) (N-terminal polypeptide fragment consisting of residues 2-114 of Sup35 spontaneously aggregates to form thin filaments in vitro.). The M domain is highly charged and therefore acts to maintain the protein in solution. This property causes the aggregation process to proceed more slowly, providing beneficial control to the system.

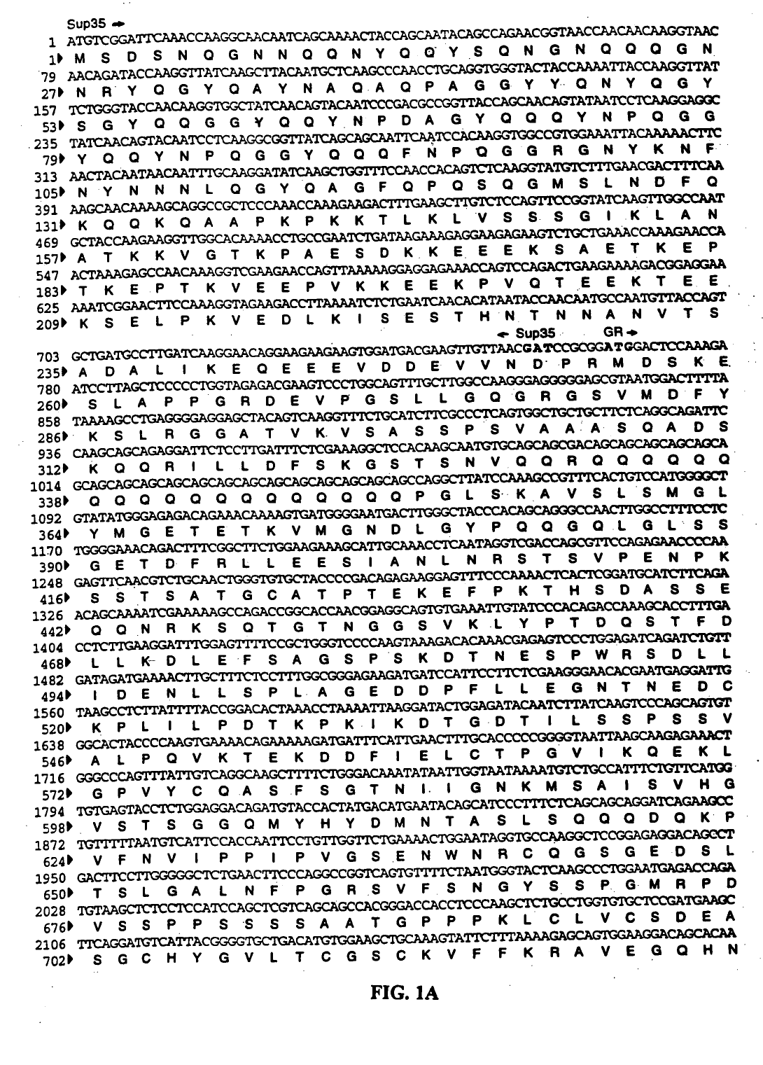

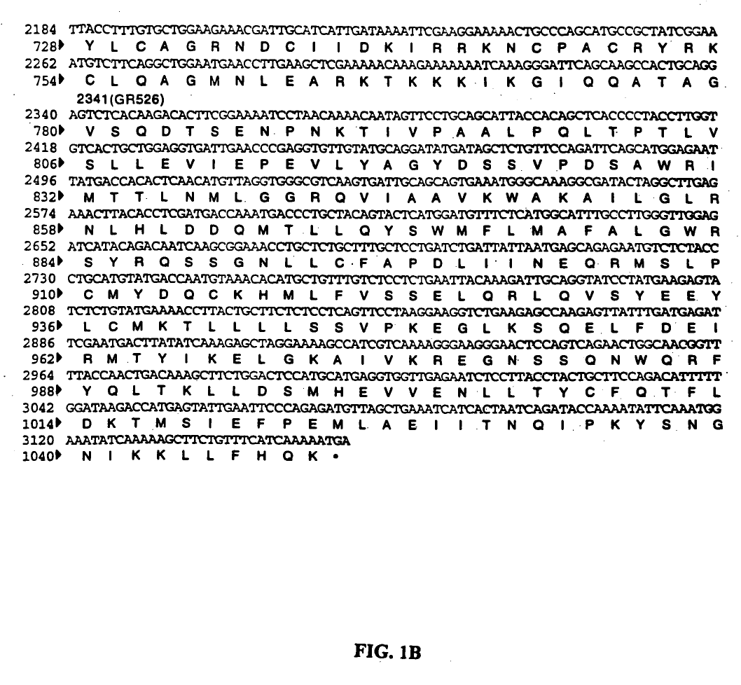

A chimeric polynucleotide FIG. 1 and (SEQ ID NO: 50) was constructed comprising a nucleotide sequence encoding the N and M domains of Sup35 (FIG. 1 and SEQ ID NO: 50, bases 1 to 759) fused in-frame to a nucleotide sequence (derived from a cDNA) encoding the rat glucocorticoid receptor (GR) (Genbank Accession No. M14053, FIG. 1 and SEQ ID NO: 50, bases 766-3150), a hormone-responsive transcription factor, followed by a stop codon. This construct was inserted into the pRS316CG (ATCC Accession No. 77145, Genbank No. U03442) and pG1 (Guthrie & Sink, “Guide to Yeast Genetics and Molecular Biology” in Methods of Enzymology, Vol. 194, pp. 389-398 (1981)) plasmids under the control of either the CUP1 promoter (plasmid pCUP1-NMGR, inducible by adding copper to the growth medium) or the constitutive GPD promoter (plasmid pGDP-NMGR). The nucleotide sequences of CUP1 and GDP (Genbank Accession No. M13807) promoters are set forth in SEQ ID NOs: 11 and 48, respectively. The GR coding sequence without NM, in the same promoter and vector constructs (plasmids pCUP1-GR and pGDP-GR), served as a control. GR activity in transformed yeast was monitored with two reporter constructs containing a glucocorticoid response promoter element (GRE) [Schena & Yamamoto, Science, 241:965-967 (1988)] fused to either a β-galactosidase (Swiss-Prot. Accession No. P00722) or to a firefly luciferase (Genbank Accession No. M15071) coding sequence. When GR is activated by hormone, e.g., deoxycorticosterone (DOC), it normally binds to the GRE and promotes transcription of the reporter enzyme in either mammals or yeast. See M. Schena and K. Yamamoto, Science 241:965-967 (1988).

B. Construction of a NMSUP35-GFP Chimeric Gene

A chimeric gene comprising the NM region of Sup35 fused to a green fluorescent protein (GFP) sequence and under the control of the CUP1 promoter was constructed essentially as described in Patino et al., Science, 273: 622-626 (1996) (construct NPD-GFP), incorporated by reference herein. (The use of GFPs as reporter molecules is reviewed in Kain et al., Biotechniques, 19:650-655 (1995); and Cubitt et al., Trends Biochem. Sci., 20:448-455 (1995), incorporated by reference herein.) The resulting construct encodes the NH₂-terminal 253 residues of Sup35 (SEQ ID NO: 2) fused in-frame to GFP. The NM-Sup35-GFP encoding sequence was amplified by PCR and cloned into plasmid pCLUC [D. Thiele, Mol. Cell. Biol., 8: 745 (1988)], which contains the CUP1 promoter for copper-inducible expression. A similar construct was created substituting the constitutive GDP promoter for the CUP1 promoter. An identical GFP construct lacking the NM fusion also was created.

C. Transformation and Phenotypic Analysis of [psi−] and [PSI⁺] Yeast

1. Constructs Regulated by the CUP1 Promoter

The GR and NM-GR constructs regulated by the CUP1 promoter on a low copy plasmid (ura selection) were transformed into [psi−] and [PSI⁺] yeast cells (strain 74D) along with a 2μ (high copy number) plasmid containing a GR-regulated β-galactosidase reporter gene with leucine selection. Transformants were selected by sc.-leu-ura and used to inoculate sc.-leu-ura medium. Cultures were grown overnight at 30° C., and induced by adding copper sulfate to the medium to a final 0-250 μM copper concentration.

After 4 to 24 hours of induction, both proteins were expressed at a similar level in [psi−] cells, and both the GR and NM-GR transformed [psi−] cells produced similar levels of reporter enzyme activity in response to hormone (DOC added to a final concentration of 10 μM at the time of copper sulfate induction). Virtually no reporter enzyme activity was detected without hormone. The fact that both GR and NM-GR constructs resulted in similar levels of activity indicates that the NM fusion does not intrinsically alter the ability of GR to function in hormone-activated transcription, demonstrating the utility of the NM domain as a fusion protein tag.

In contrast, when the same constructs were transformed into yeast cells that contain the heritable, conformationally-altered form of Sup35 [PSI⁺], GR activity was reduced in cells expressing the NM-GR fusion construct, compared to cells expressing GR. Thus, pre-existing prions (which comprise self-coalescing aggregates of NM-containing Sup35 protein) can interact with NM-GR. Similar results were obtained with NM-Green Fluorescent Protein (GFP) constructs: NM-GFP interacted with pre-existing [PSI⁺] elements, but GFP alone did not.

An important difference existed between the NM-GR and NM-GFP studies in the [PSI⁺] cells, however. Unlike the NM-GR fusion, the NM-GFP fusion retained similar GFP activity with the [PSI⁺] prion, i.e., the NM-GFP fusion still glowed green. This difference in activity is explained by the facts that, for biological activity, GR needs to be in the nucleus, bind to DNA, and interact in specific ways with other elements of the transcription machinery. When NM-GR is sequestered in [PSI⁺] cells by interacting (aggregating) with the Sup35 prion filaments, the GR function is diminished.

2. Constructs Regulated by the Constitutive GPD Promoter on a High Copy Plasmid.

A set of experiments demonstrated that plasmids that cause expression of NM at a high level can be successfully transformed into [psi−] yeast cells, but not into [PSI⁺] cells. Apparently, over-expressed NM causes excessive prion-like aggregation of endogenous Sup35 in cells that are already [PSI⁺], eliminating so much translation termination factor function that the yeast cells cannot survive.

When a high copy plasmid vector comprising the NM-GR open reading frame under the control of the constitutive GPD promoter was used to transform [psi−] or [PSI⁺] yeast, no [PSI⁺] transformants were obtained, whereas [psi−] transformants were readily obtained. The control GR construct in the same vector and under control of the same promoter transformed equally well into both [PSI⁺] and [psi−] cells.

When amino acids 22-69 in the N domain of Sup35 are deleted, the resultant protein fails to form ordered aggregates, and yeast comprising this Sup35 variant fail to adopt a [PSI⁺] phenotype. When these same amino acids were deleted from the high copy number NM-GR plasmid, the inability to transform [PSI⁺] cells was eliminated: transformants were obtained as readily in [PSI⁺] as [psi−] cells.

Both NM-GR and GR [psi−] transformants were used to inoculate sc.-leu-trp medium, and the cultures were grown at 30° C. overnight, diluted into fresh medium to achieve a cell density of 2-4×10⁶ cells/ml, induced with DOC (10 μM final concentration), and grown for an additional period varying from 1 hour to overnight. Analysis of marker gene activity in the transformed [psi−] cells demonstrated that hormone responsive transcription was lower in NM-GR transformants than in GR transformants. Western blotting using an anti-GR monoclonal antibody (Affinity Bioreagents Inc., MA1-510) was used to examine the levels of NMGR and GR expression in these cells. Although cells carrying the NM-GR fusion had lower levels of GR activity, the NM-GR protein was actually expressed at a much higher level than the GR protein without the NM domain. Thus, the reduced levels of hormone-activated transcriptional activity were not due to an effect of NM on the accumulation of the transcription factor, but to an alteration in GR activity in the NM-GR-expressing cells. This reduced activity suggested that NM-GR is capable of undergoing a de novo, prion-like alteration in function when it is expressed at a sufficiently high level.

To confirm that NM-GR was forming prions de novo in the transformed [psi−] cells into which it had been introduced, such cells were induced with copper to express NM-GR and then were plated onto copper-free media lacking adenine, and therefore selective for the [PSI⁺] element/phenotype. See Chernoff et al., Science, 268: 880 (1995), and Cox et al., Yeast, 4(3): 159-178 (1988). A substantial fraction of the cells were able to grow on medium selective for [PSI⁺], suggesting that the highly expressed NM-GR was responsible for the formation of new prions putatively containing both NM-GR and Sup35 protein. Moreover, the number of colonies obtained varied with the level of copper induction prior to plating. This change in the growth properties of the cells was observed to be heritable and was maintained even under conditions where the NM-GR plasmid construct was lost by the host cells, indicating that NM-GR had induced the formation of a new Sup35-containing prion.

D. Analysis of NMGR-induced Phenotype in Cells Carrying a Deletion of the NM Region of Sup35.

To further confirm that NM-GR was truly functioning as an independent, novel prion, experiments were conducted to determine whether an NM-GR prion was formed independently of both the yeast [PSI⁺] element and the endogenous Sup35 protein. Specifically, the GPD-regulated GR and NM-GR constructs were co-transformed with plasmid p5275 (containing GRE linked to a firefly luciferase reporter gene) into a yeast strain (ΔNMSUP35) carrying a deletion of the NM region of the SUP35 gene. Three independent transform ants of each construct (GR or NM-GR) were examined. Colonies were picked and grown overnight in SC selective media (-trp, -ura) at 30° C. Thereafter, deoxycorticosterone (DOC) was added to the growth medium to a final concentration of 10 μM. Luciferase activity was assayed in intact cells after 25 hours of DOC induction.

All three transformants expressing the NM-GR protein showed lower levels of GR activity (specific activities of about 4, 5, 4) than the three transfornants expressing GR without the NM fusion (specific activities of about 23, 28, and 39). The differences in GR activity was observed after 1 hour of hormone induction and appeared to increase after 5.5 or after 25 hours of induction.

Western blotting was conducted to determine whether the differences in activity were the result of differences in protein concentration. Ethanol lysates were prepared from 3 ml yeast cultures expressing GR or NMGR twenty-five hours after the addition of DOC. About 50 μg total protein was analyzed by SDS/PAGE and immuoblot. The protein gel was transferred onto PVDF membranes and probed with a monoclonal antibody against GR (Bu-GR2, Affinity Bioreagents, Golden Colo.). The same membrane was later stained with Coomassie blue to semiquantitatively evaluate total protein. The Western studies again showed that the levels of NM-GR were higher than the levels of GR alone.

E. Effect of Guanidine Hydrochloride and Hsp104 on NM-GR Prions.

When the yeast having [URE3] or [PSI⁺] phenotypes are passaged on medium containing low concentrations of guanidine hydrochloride (GdHCl), their prion determinants change (“cure”) at a high frequency from the aggregated, inactive prion state into the active, unaggregated state, and such changes are heritable. These phenotypes also can be cured by over-expression of the chaperone Hsp104.

Another series of experiments were conducted to assay for such curative behavior in yeast harboring an NM-GR construct. The natural GR protein contains a ligand-binding domain and hormone must be added to the medium to determine whether or not the protein is active. For this series of experiments, the hormone-binding domain was removed from the NM-GR construct, creating an NM-GR fusion that was constitutively active.

Yeast expressing the NM-GR chimeric construct and a glucocorticoid response element fused to a β-galactosidase marker exhibited different levels of prion-like behavior, manifested by different colony colors. In addition to white colonies (indicative of a prion-like state lacking β-gal induction) and blue colonies (indicative of soluble NM-GR and high levels of β-gal induction), medium blue and pale blue colonies also were observed. (Western blotting indicated that differently colored colonies contained comparable amounts of GR protein.) These differently colored colonies were replica-plated onto plates containing 5 mM GdHCl and then subsequently replica-plated again onto X-Gal indicator plates. In control cells expressing vector alone (no NM-GR insert), white colonies remained white. However, all of the NM-GR-expressing colonies produced blue colonies. The efficiency of curing varied with the NM-GR strain: medium blue colonies produced almost entirely blue colonies, whereas pale blue colonies produced a mixture of blue and white colonies.

To determine if the heritable loss of NM-GR activity is susceptible to Hsp104 curing, white colonies of cells expressing NM-GR were transformed with a GDP-HSP104 over-expression plasmid and streaked onto X-Gal indicator plates. Control cells transformed with empty vector remained white. In contrast, white cells transformed with the Hsp104 over-expression construct changed to blue. The blue cells remained blue upon-restreaking, indicating that transient over-expression of Hsp104 was sufficient to cure cells of the heritable reduction of NM-GR activity.

When the same NM-GR constructs were used to transform yeast containing a deletion mutation of Hsp104, white colonies were never produced. This finding is consistent with the observation that Hsp104 mutations are incompatible with the maintenance of the [PSI⁺] phenotype.

Together, the foregoing data indicate that the difference in GR activity observed when NM-GR is expressed at a high constitutive level is due to a heritable alteration in GR function, rather than to an alteration in GR expression.

Collectively, the foregoing experiments demonstrate that the amino-terminal domain of a prion-like yeast gene, Sup35, can be fused to a polypeptide from a wholly different protein to construct a novel, chimeric gene and protein having prion-like properties. Significantly, these results are believed to be the first demonstration that a SCHAG protein domain can be fused to a non-native protein domain to form a chimera, expressed in a host cell thatfails to express the native SCHAG protein, and still behave in a prion-like manner. (Specifically, these results demonstrate that the NM domains of SUP35 will behave like a prion even when the C-terminal domain of the protein is not the native Sup35 C-terminus, and even when the host cell does not express an endogenous Sup35 protein containing an NM region.) The experiments also define exemplary assays for screening other putative prion-like peptides for their ability to confer a prion-like phenotype. (It will be apparent that the use of markers other than GFP, GR, luciferase, or β-galactosidase would work in such assays. The GFP marker is useful insofar as it provides an effective marker for localizing a fusion protein in vivo. The GR marker is additionally useful insofar as GR activity depends on GR localization in the nucleus, DNA binding, and interaction with transcription machinery; whereas GFP is active in the cytoplasm.) Exemplary prion-like peptides for screening in this manner are peptides identified according to assays described below in Example 5; mammalian PrP peptides responsible for prion-forming activity; and other known fibril-forming peptide sequences, such as human amyloid β (1-42) peptide.

In addition, the experiments demonstrate an improved procedure for recombinant production of certain proteins that might otherwise be difficult to recombinantly produce, e.g., due to the protein's detrimental effect on the growth or phenotype of the host cell. For example, DNA binding and DNA modifying enzymes that might locate to a cell's nucleus and detrimentally effect a host cell may be expressed as a fusion with a SCHAG amino acid sequence from a prion-like protein. In host cells wherein the aggregate-forming phenotype is present, the recombinant protein is “sequestered” into higher order aggregates. By virtue of this sequestration, the biological activity of the resultant protein in the nucleus is reduced. The fusion protein is purified from the insoluble fraction of host cell lysates, and can be cleaved from the fibril core if an appropriate endopeptidase recognition sequence has been included in the fusion construct between the SCHAG amino acid sequence and the sequence of the protein of interest. (An appropriate endopeptidase recognition sequence is any recognition sequence that is not present in the protein of interest, such that the endopeptidase will cleave the protein of interest from the fibril structure without also cleaving within the protein of interest.)

### EXAMPLE 2

### Construction and Assaying of a Chimeric, Prion-like Gene and Protein with Yeast Ure2 Protein

The following experiments were performed to demonstrate that the prion-determining domain of yeast Ure2 protein also can be fused to a polypeptide other than the Ure2 functional domain to construct a novel, chimeric gene and protein having some prion-like properties. Two prion-like elements are known in yeast: [PSI⁺] and [URE3]. The underlying proteins, Sup35 and Ure2, each contain an amino-terminal domain (the N domain) that is not essential for normal function but is crucial for prion formation. The N domains of both Sup35 and Ure2 are unusually rich in the polar amino acids asparagine and glutamine.

A. Construction of a NUre2-CSup35 Chimeric Gene

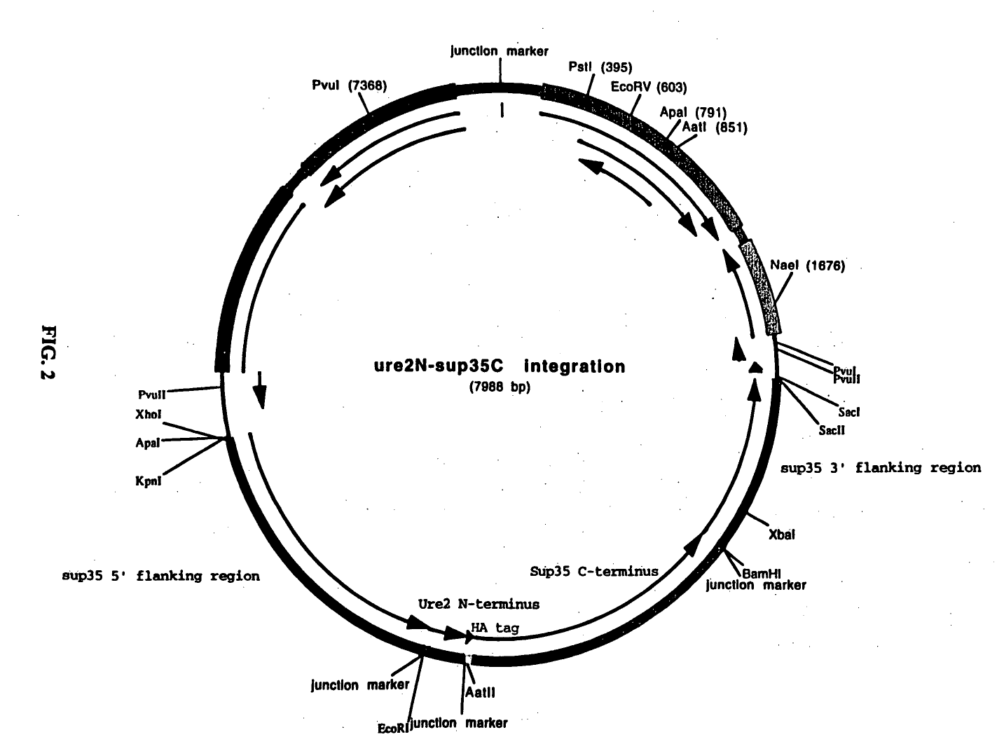

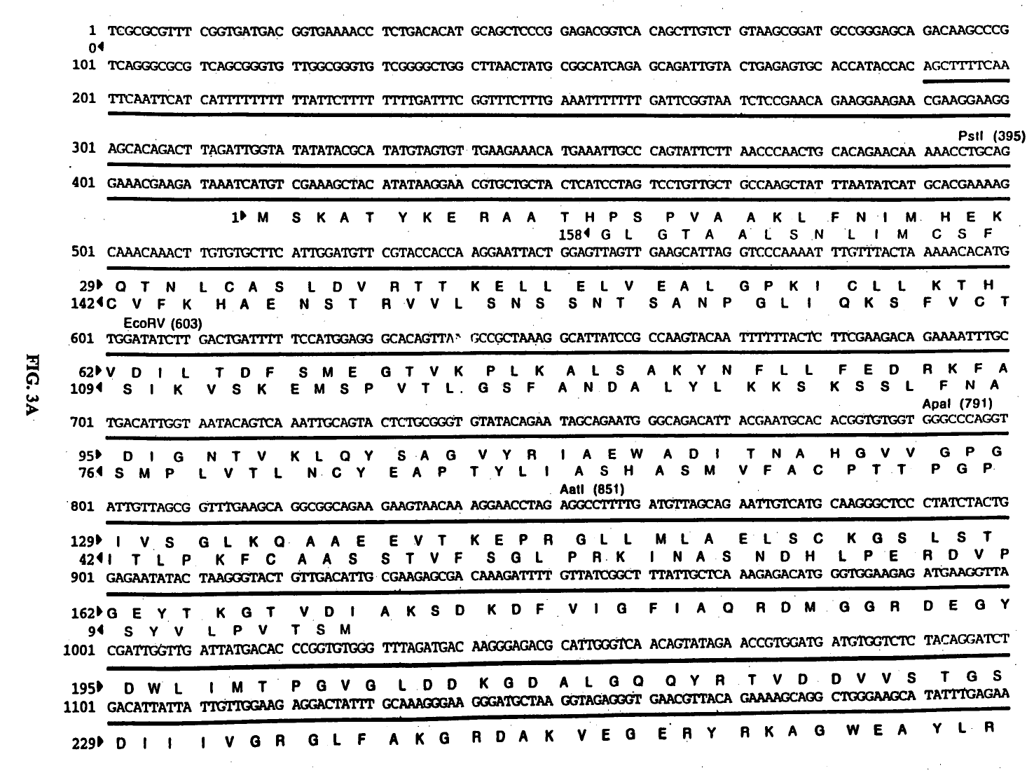

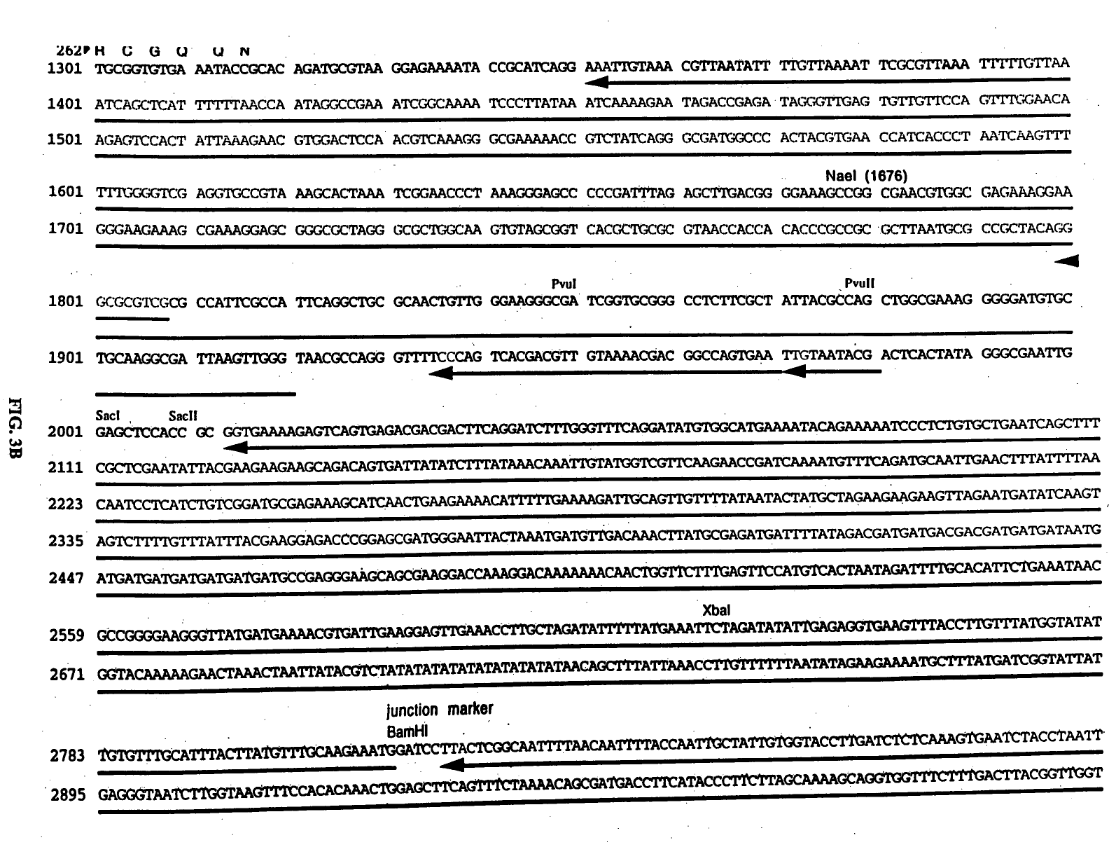

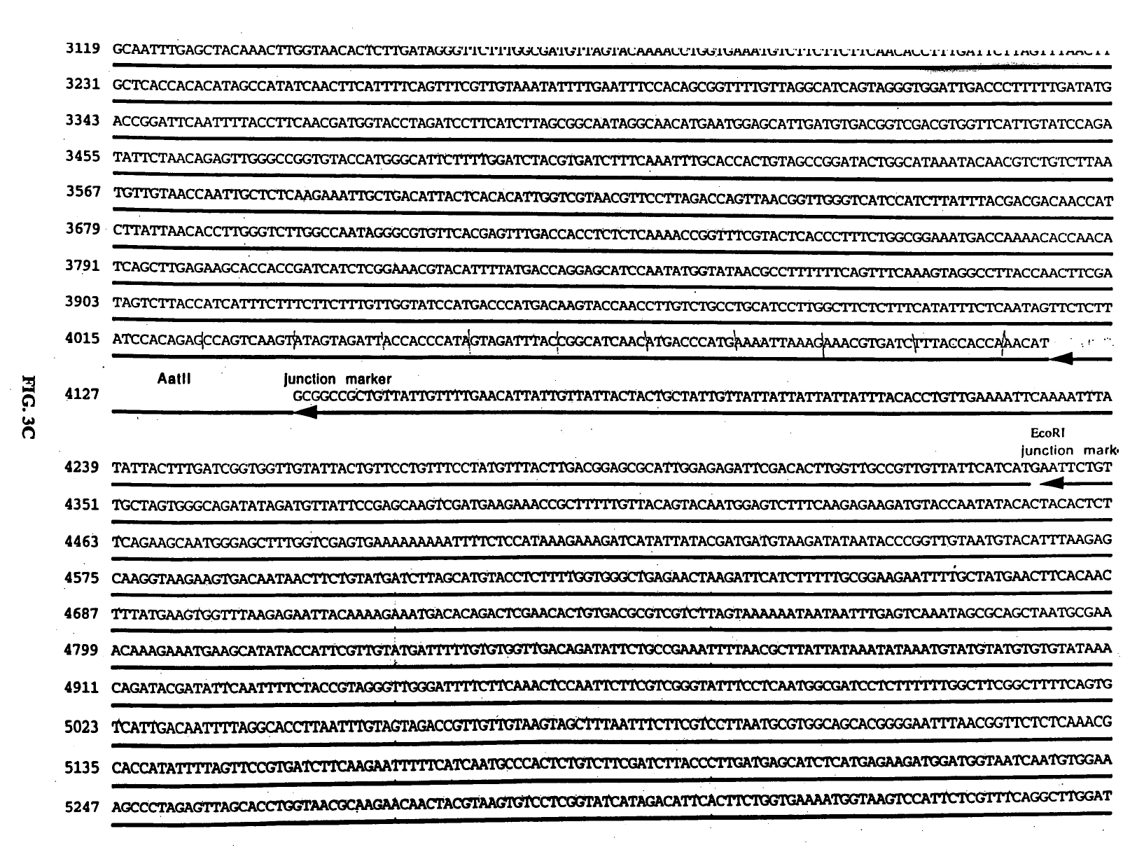

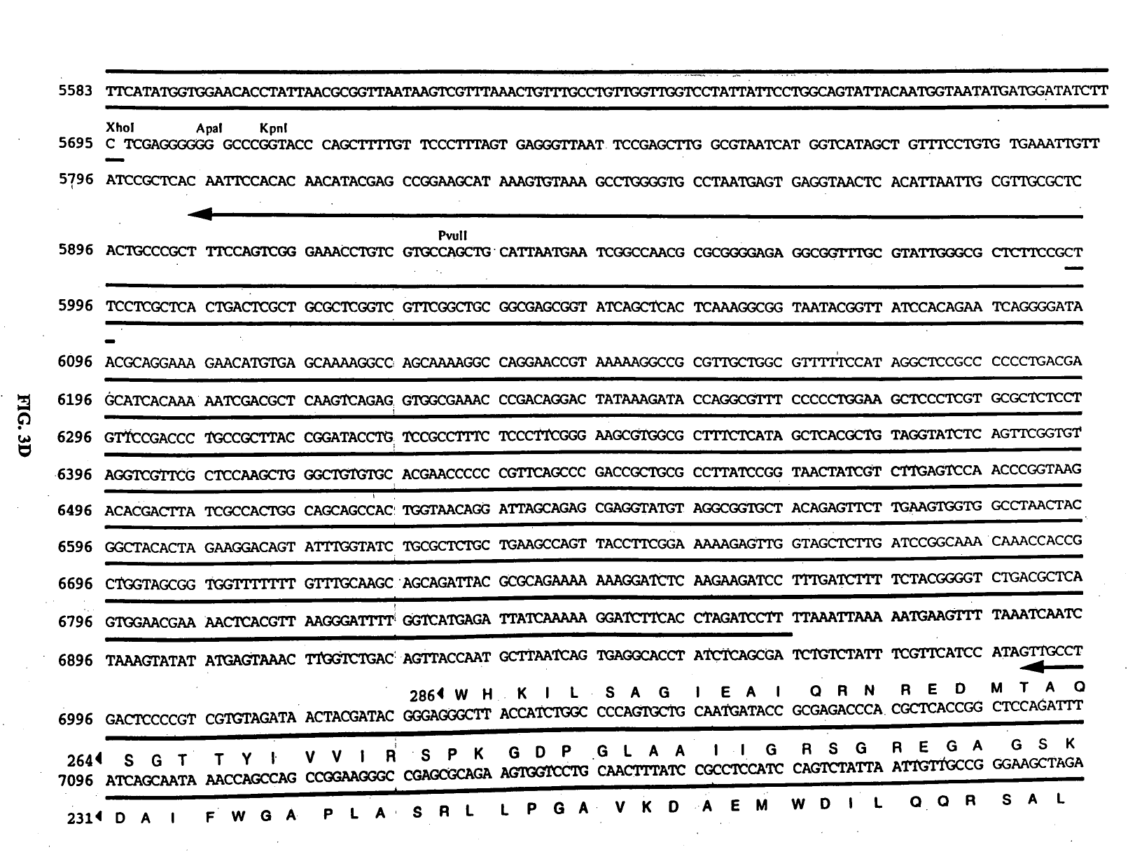

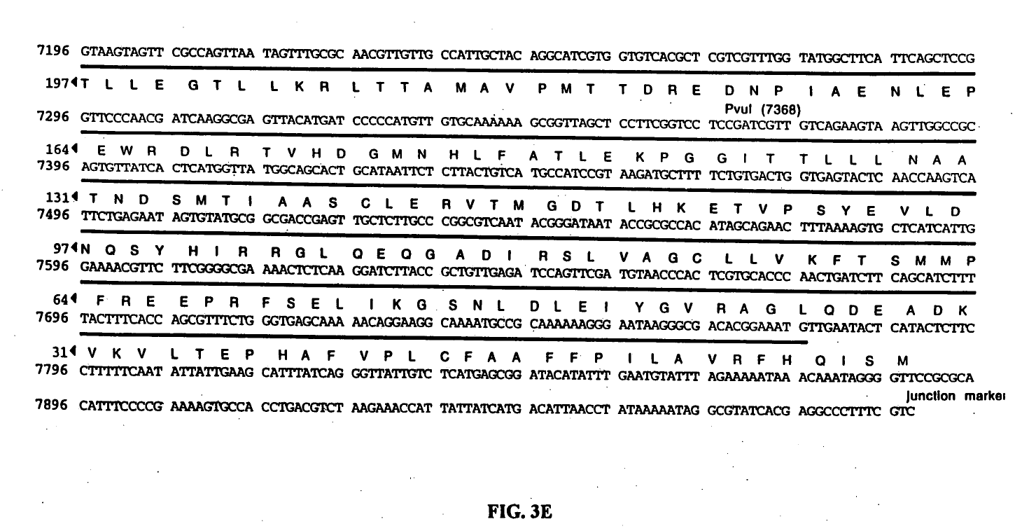

A chimeric polynucleotide (FIG. 3, SEQ ID NO: 49) was constructed comprising a nucleotide sequence encoding the N domain of yeast (Saccharomyces cerevisiae) Ure2 protein (Genbank Accession No. M35268, SEQ ID NO: 3, bases 182 to 376, encoding amino acids 1 to 65 (SEQ ID NO: 4) of Ure2 (NUre2)), fused in-frame to a nucleotide sequence encoding a hemagglutinin tag (SEQ ID NO: 13, TAC CCA TAC GAC GTC CCA GAC TAC GCT), fused in-frame to a nucleotide sequence encoding the C domain of yeast Sup35 (CSup35) protein that is responsible for translation-regulation activity of Sup35 (Genbank Accession No. M21129, SEQ ID NO: 1, bases 1498-2793, encoding amino acids 254 to 685 of Sup35 (SEQ ID NO: 2)). At the 5′ and 3′ ends of this construct were 5′ and 3′ flanking regions, respectively, of the yeast Sup35 genomic DNA. This construct was inserted into the pRS306 plasmid (available from the ATCC, Manassas, Va., USA, Accession No. 77141; see also Genbank Accession No. U03438) as shown in FIGS. 2 and 3, and used to transform yeast as described below.

B. Transformation and Phenotypic Analysis of Yeast

To replace the Sup35 gene with the NUre2-CSup35 chimeric gene, the first step was to integrate the gene fragment into the yeast genome. Freshly grown cells from overnight culture were collected and resuspended in 0.5 ml LiAc-PEG-TE solution (40% PEG4000, 100 mM Tris-HCL, pH7.5., 1 mM EDTA) in a 1.5 ml tube. 100 μg/10 μl carrier DNA (salmon testis DNA, boiled 10 minutes and chilled immediately on ice) and 1 μg/2 μl of transforming plasmid DNA were added and mixed. This transformation mixture was incubated overnight at room temperature and then heat shocked at 42^{□}C for 15 minutes. 100 μl of transformation mixture were then spread onto a uracil dropout plate. After transformation, selection for Ura+ results in an integration event, such that native and chimeric genes bracket the URA3-containing plasmid sequence. Transformants were picked and cells having the integrated chimeric gene were confirmed by genomic PCR and Western blot.

The second step of the replacement involved the excision or “popping out” of the wildtype Sup35 gene through homologous recombination between the native Sup35 and the chimeric sequence. Popout of the plasmid was monitored by screening for colonies that are ura− and therefore resistant to the drug 5-fluoroorotic acid (5-FOA). Cells with NUre2-CSup35 integrated were thus plated onto 5-FOA medium to select for those that have the plasmid sequence containing one copy of the Sup35 gene popped out. Clones in which the native Sup35 gene had been replaced with the chimeric gene were then screened by means of colony PCR and further confirmed by Western blot.

To screen for yeast strains that have gene integration and replacement, a Ure2 coding sequence N-terminal primer and a Sup35 coding sequence primer were used for PCR reactions. The NUre2-CSup35 DNA fragment can only be amplified from genomic DNA of cells containing the chimeric gene. To confirm that only the fusion protein of NUre2-CSup35 was expressed in those cells that have the gene replacement, yeast cells were lysed and the cell lysates were run on SDS-polyacrylamide gel and proteins were transferred to PVDF immunoblot. Since there is a hemagglutinin (HA) tag inserted between NUre2 and CSup35, Western blots were then probed with anti-HA antibody from Boehringer Mannheim. To confirm that NUre2-CSup35 is the only copy of Sup35 gene in yeast genome, Western blots were also probed with an antibody against the middle region of Sup35 protein. Loss of antibody signal verified that the NM region of Sup35 gene had been replaced with the N-terminus of Ure2. Thus, the transformed cells were characterized by a deleted native Sup35 gene that had been replaced by the NUre2-CSup35 chimeric gene.

Transformed colonies carrying the chimeric NUre2-CSup35 gene of interest were grown on rich medium (YPD) at 30° C. The resultant colonies were streaked onto [PSI⁺] selective medium (SD-ADE) and incubated at 30° C. to determine whether some or all contained a [PSI⁺] phenotype. Two different types of colonies were observed. Some showed normal translational termination characteristic of a [psi−] phenotype. Others showed the suppressor phenotype characteristic of [PSI⁺] cells. Both phenotypes were very stable and were inherited from generation to generation of the transformed yeast cells.

To determine whether the observed difference in translational fidelity was due to a heritable change in protein conformation, cells were lysed and the lysates subjected to centrifugation at 12,000 or 100,000×g for 10 minutes. Supernatants and precipitate fractions were screened for the fusion protein using an anti-HA antibody (HA 11, Covance Research Products Inc.). The cells that showed reduced translational fidelity also showed aggregation of the NUre2-CSup35 fusion protein, whereas the fusion protein did not appear aggregated in cells having normal translation termination characteristics.

The foregoing experiments demonstrate that the amino-terminal domain of another prion-like yeast gene, Ure2, can be fused to a polypeptide derived from a wholly different protein to construct a novel, chimeric gene and protein having prion-like properties. These results represent the first such demonstration of this kind. [Compare Maison & Wickner, Science, 270: 93 (1995) (Ure2₁₋₆₅/β-gal fusion did not change the activity of the β-galactosidase enzyme) and Paushkin et al., EMBO J., 15(12): 3127-3134 (1996) (GST-NSup35 chimeric construct did not allow native Sup35 to adopt an altered state.)]

Several factors are suggested for achieving prion-like behavior with chimeric genes that comprise SCHAG sequences. First, it is preferable to include the SCHAG sequence at a location in the chimeric gene (e.g., amino-terminus or carboxy-terminus) that corresponds to the location at which it is found in its native gene. For example, if NSup35 is selected as the SCHAG sequence, then the chimeric gene preferably is constructed with NSup35 at the amino-terminus, preceding the sequence encoding the polypeptide of interest. Second, it is preferable to include a spacer region of, e.g., at least 5, 10, 20, 30, 40, or 50 amino acids, and preferably at least 60, 70, 80, 90, 100, 120, 130, 140, or 150 amino acids, to separate the SCHAG domain from other domains and reduce the likelihood of steric hinderance caused by other domains. The length of spacer apparently can be quite large because a chimeric construct comprising whole Sup35 fused to Green Fluorescence Protein appears to act as a prion in preliminary experiments. Third, it is preferable if the protein of interest is a protein that does not itself naturally form multimers, because multimer formation of the protein of interest is apt to cause steric interference with the ordered aggregation of the SCHAG domain. (Maison & Wickner's research involved β-galactosidase, which forms a tetrameric functional unit.) The experiments also demonstrate an alternative assay system (i.e., CSup35 fusions) to the GFP and GR assay systems described in the preceding example to screen peptide sequences for their ability to confer prion-like phenotypic properties.

Also contemplated are fusion proteins comprising the M domain of Sup35, or portions of fragments thereof, fused to a different protein to generate a novel protein with prion-like activities. Likewise, fusion proteins displaying prion-like properties, comprising portions or fragments of the N domain, or comprising portions or fragments of the N and of the M domain are also contemplated.

### EXAMPLE 3

### Modulation of Propensity of Protein to Form Prion-like Aggregates

The following experiments demonstrate that the propensity of novel chimeric proteins to aggregate into prion-like fibrils can be modulated by varying the number of oligopeptide repeats in the SCHAG portion of the chimeric protein. An increased propensity to form such fibrils is useful in instances where the fibrils themselves comprise a desirable end product to be harvested from cells, e.g., via lysis and centrifugation; and in instances where fibril formation in vivo is desired to phenotypically alter a cell, e.g., by sequestering a biologically active molecule in the cell away from the molecule's normal subcellular region of biological activity.

The yeast Sup35 protein contains an oligopeptide repeat sequence (PQGGYQQYN, SEQ ID NO: 2, residues 75 to 83; with imperfect repeats at residues 41 to 50; 56 to 64; 65 to 74; and 84 to 93). The following experiments demonstrated that an expansion of this oligopeptide repeat in the NM region of Sup35 increases the rate of appearance of new, heritable, [PSI⁺]-like elements, whereas decreasing the number of repeats lessened the rate of appearance of such elements.

Three expression vectors were created for the experiment containing a chimeric gene comprising a CUP1 promoter sequence (SEQ ID NO: 11) operably linked to a sequence encoding a Sup35 NM region, fused in-frame with a “superglow” GFP encoding sequence (SEQ ID NO: 39). In the first construct (R_{Δ}2-5), the Sup35 NM region had been modified by deleting four of the five oligopeptide repeats found in the native N region (SEQ ID NOs: 14 & 15). In the second construct (R2E2), the Sup35 NM region had been modified by twice expanding the second oligopeptide repeat found in the native N region, creating a total of seven oligopeptide repeats (SEQ ID NOs: 16 & 17). In the third construct, the native Sup35 NM region was employed (SEQ ID NO: 1, nucleotides 739 to 1506, encoding residues 1 to 256 of SEQ ID NO: 2). The CUP1 promoter permitted control of the expression of the chimeric proteins by manipulation of copper ion concentration in the growth medium. [See Thiele, D. J., Mol. Cell. Biol., 8: 2745-2752 (1988).] The attachment of GFP to NM permitted visualization of the mutant proteins in living cells.

Each of the three above-described NM-GFP constructs were introduced via homologous recombination at the site of the wild-type Sup35 gene into [psi−] yeast cells carrying a nonsense mutation in the ADEI gene (strain 74-D694 [psi−]), and monitored for the frequency at which cells converted to a [PSI⁺] phenotype. Cell cultures in the log phase of growth at 30° C. were induced to express the GFP-fusion proteins by adding CuSO₄ to the cultures cells to a final concentration of 50 μM. For analysis via fluorescence microscopy, cells were fixed with 1% formaldehyde after four hours and twenty hours of culture. For analysis of [PSI⁺] induction, cells over-expressing the GFP fusion proteins were serially diluted and spotted onto YPD and SD-ADE media after four hours and twenty hours. Conversion was measured by the ability of cells to grow on medium without adenine (SD-ADE). The [PSI⁺] phenotype causes readthrough of nonsense mutations, producing sufficient protein to suppress the ADEI mutation and allow growth without adenine.

Cells were induced with copper for 4 hours to promote expression of the chimeric gene and serially diluted, and then aliquots of each dilution were plated on SD-ADE, conditions that allowed loss of the plasmid. To demonstrate that the initial cultures contained similar numbers of cells, serial dilutions from each culture also were plated on rich medium (YPD) which allowed the growth of all cells in the culture. After incubating the plates for 48 hours at 30° C., colonies on each plate were counted.

Cells expressing the oligopeptide repeat expansion mutation converted to [PSI⁺] at a much higher frequency than cells expressing the native Sup35NM-GFP, which in turn converted to [PSI⁺] at a higher frequency than cells expressing the oligopeptide repeat deletion mutation. The observed conversion results were specifically attributable to the production of the chimeric proteins, because the conversion to [PSI⁺] did not occur in cells that were not induced with copper (control).

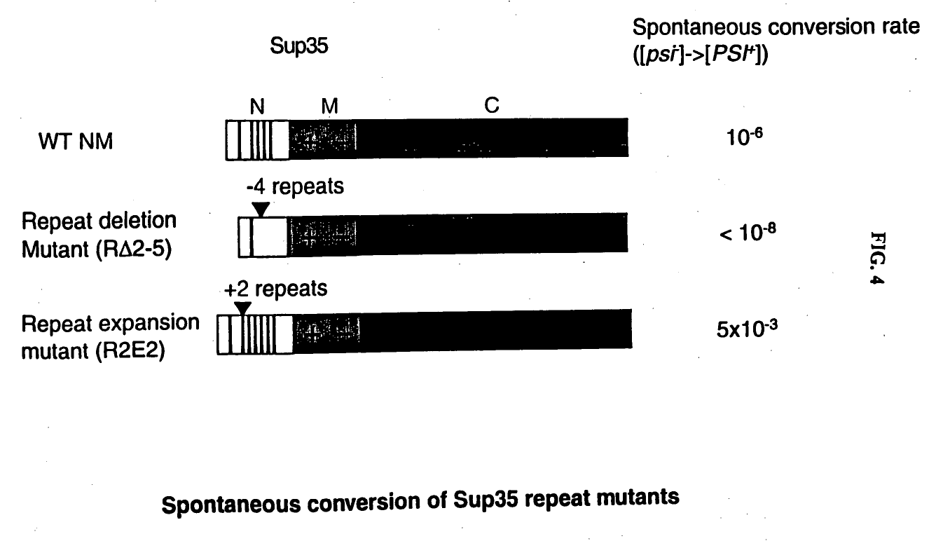

In a related experiment, the repeat expansion and repeat deletion mutations were introduced into a full-length Sup35 protein-encoding sequence to create constructs encoding the NM(R2E2) and NM(RΔ2-5) fused to the CSup35 domain. These constructs were introduced into the genome of [psi−] yeast strain 74-D694 with the wild-type Sup35 promoter, in each case replacing the native Sup35 gene. Transformants were selected on uracil-deficient medium and confirmed by genomic PCR. Recombinant excision events were selected on medium containing 5-fluoroorotic acid. [See Ausubel et al., Current Protocols in Molecular Biology, Green Publishing Associates and Wiley Interscience, New York (1991).] Strains in which wild-type Sup35 was replaced with the R2E2-CSup35 and RΔ2-5CSup35 variants were screened by PCR and confirmed by Western blotting. The cells were cultured on ypd or synthetic complete media at 25° C. for 24 hours, serially diluted, and plated on SD-ADE media to screen for [PSI⁺] conversions. As shown in FIG. 4, the spontaneous rate of appearance of [PSI⁺] colonies was increased about 5000-fold in cells carrying the repeat expansion (R2E2) compared to wild-type cells. The wild-type cells produced colonies on the selective medium at a frequency of about 1 per million cells plated. The RΔ2-5 cells produced such colonies at even lower frequency, and it appears that none of these were attributable to development of a [PSI⁺] phenotype, since they could not be cured by growth on medium containing 5 mM guanidine HCl. In contrast, growth of the wild-type and the R2E2 colonies on the selective medium could indeed be cured by the guanidine HCl treatment.

In additional experiments, the effects of the Sup35 repeat variants were examined when they were used to replace the wild-type Sup35 gene in [PSI⁺] cells. Cells with the R2E2 replacement remained [PSI⁺], whereas all cells carrying the RΔ2-5 replacement became [psi−]. Thus, maintenance of the [PSI⁺] phenotype requires a Sup35 gene having more than one of the oligopeptide repeats.

Still another series of tests examined the effects of the repeat variants on the structural transition of NM in vitro. When purified recombinant NM is denatured and diluted into aqueous buffers, it slowly changes from a random coil into a β-sheet rich structure and forms fibers that bind Congo red with the spectral shift characteristic of amyloid proteins. When deposited at high concentrations, the Congo red-stained fibers also show apple-green birefringence. To determine if the repeat variants alter the intrinsic capacity of the protein to fold in this form, the wild-type and two repeat variants were purified in fuilly denatured states and then diluted into a non-denaturing buffer. Structural changes were monitored by the binding of Congo red [Klunk et al., J. Histochem. Cytochem., 37: 1293-1297 (1989)]and confirmed by circular dichroism and electron microscopy analysis. In these experiments, the R2E2 variant converted to a β-sheet rich structure about twice as quickly as the wild-type NM polypeptide, which in turn converted significantly faster than the RΔ2-5 variant. These differences were reproducibly obtained in both rotated and unrotated reactions, although the transition was slower in the unrotated reactions. This data indicates that alterations in the number of repeat units alters the propensity of Sup35 NM polypeptides to progress from an unfolded state into a β-sheet rich, higher-ordered structure.

The foregoing experiments demonstrate that the propensity of novel chimeric proteins to aggregate into prion-like fibrils can be modulated by alteration of the SCHAG amino acid sequence of the chimera. Modulation of any SCHAG amino acid sequence in this manner is specifically contemplated as an aspect of the invention, as are the resulting gene and protein products. In addition to alteration by adding or deleting oligopeptide repeat regions, alterations by adding or deleting larger regions is specifically contemplated as an aspect of the invention. By way of example, the entire N terminal region of Sup35 or Ure2 could be duplicated to increase the propensity of transformed cells to produce aggregated chimeric sequences.

### EXAMPLE 4

### Demonstration That a Prion Can Be Moved From One Organism to Another

The following experiments demonstrate that a prion protein from one organism will continue to behave in a prion-like manner when recombinantly expressed in another organism, and can even do so when expressed in a different cellular compartment than that in which the protein is produced in its native host.

Polynucleotides encoding mouse (SEQ ID Nos: 18 and 19) and Syrian Hamster (SEQ ID Nos: 20 and 21) PrP proteins were expressed in yeast cells under the control of the constitutive GPD promoter. The protein was produced in the yeast cytosol, without signal sequences that would normally guide it to the endoplasmic reticulum, and without the tail that is normally clipped off during maturation of these proteins in their native hosts. In other words, the PrP protein product in yeast was similar to the final mature product in mammalian neurons, except that it did not contain the sugar modification and GPI anchor. There has been considerable data suggesting that these sugar and GPI anchor characteristics are not required for prion formation.

The normal cellular form of PrP (PrP^{C})is detergent soluble, but the conformationally changed-protein that is characteristic of neurodegenerative prion disease states (PrP^{sc}) is insoluble in detergent such as 10% Triton. When PrP protein is expressed in yeast, is was insoluble in non-ionic detergents, suggesting that a PrP^{sc} form was present.

PrP-transfected yeast cells were lysed in the presence of 10% Sarkosyl and centrifuged at 16,000×g over a 5% sucrose cushion for 30 minutes. Proteins in both the supernatant and pellet fractions were analyzed on SDS polyacrylamide gels. Coomassie blue staining revealed that most proteins were soluble under these conditions and were present in the supernatant fraction. When identical gels were blotted to membranes and reacted with antibodies against mammalian PrP, most of the PrP protein was found in the pellet fraction, further suggesting that a PrP^{sc} form was present in the yeast.

Protease studies provide further evidence that the yeast PrP was adopting a PrP^{sc} conformation. When PrP protein is expressed in yeast it displays the same highly specific pattern of protease digestion as does the disease form of the protein in mammals. The normal cellular form of PrP is very sensitive to protease digestion. In the disease form, the protein is resistant to protease digestion. This resistance is not observed across the entire protein, but rather, the N-terminal region from amino acids 23 to 90 is digested, while the remainder of the protein is resistant. As expected, when PrP was expressed in the yeast cytosol it was not glycosylated, and it migrated on an SDS gel as a protein of ˜27 kD. After protease digestion, a resistant fragment of ˜19-20 kD was detected, corresponding exactly to the size expected if the protein were being cleaved at the same site as the PrP^{sc} form of the protein that can be recovered from diseased mammalian brains.

The foregoing data indicates that, when mammalian PrP is expressed in yeast, a species from an entirely different taxonomic kingdom, it be behaves unlike common yeast proteins, and very much like the disease form of PrP in mammals.

Besides the diseased form, a small portion of PrP protein expressed in yeast cytosol also behaves like the normal cellular form of PrP. Even after centrifugation at 180,000 g for 90 minutes, there is still some PrP protein detectable in the supernatant fraction. This part of PrP expressed in yeast, like normal cellular PrP, was soluble in non-ionic detergent, suggesting this small portion of PrP is present in the PrP^{c} conformation.

### EXAMPLE 5

### Assays to Identify Novel Prion-like Amyloidogenic Sequences

The following experiments demonstrate how to identify novel prion-like amyloidogenic sequences and confirm their ability to form prions in vivo. The experiments involve (A) identifying sequences suspected of having prion forming capability; and (B) screening the sequences to confirm prion forming ability.

A. Identifying sequences suspected of having prion forming capability

Known prion or prion-like amino acid sequences, or polynucleotides encoding such sequences, are used to probe sequence databases or genomic libraries for similar sequences. For example, in one embodiment, a prion or prion-like amino acid sequence (e.g., a mammalian PrP sequence; the N or NM regions from a yeast Sup35 sequence; or the N region from a yeast Ure2 sequence) is used to screen a protein database (e.g., Genbank or NCBI) using a standard search algorithm (e.g., BLAST 1.4.9.MP or more recent releases such as BLAST 2.0, and a default search matrix such as BLOSUM62 having a Gap existence cost of 11, a per-residue gap cost of 1, and a Lambda ratio of 0.85. See generally Altschul et al., Nucleic Acids Res., 25(17): 3389-3402 (1997).). As an exemplary cutoff, database hits are selected having P(N) less than 4×10⁻⁶, where P(N) represents the smallest sum probability of an accidental similarity. For database searching, polypeptide sequences are preferred, but it will be apparent that polynucleotides encoding the anino acid sequences also could be used to probe nucleotide sequence databases.

In an alternative embodiment, one or more polynucleotides encoding a prion or prion-like sequence is amplified and labeled and used as a hybridization probe to probe a polynucleotide library (e.g., a genomic library, or more preferably a cDNA library) or a Northern blot of purified RNA for sequences having sufficient similarity to hybridize to the probe. The hybridizing sequences are cloned and sequenced to determine if they encode a candidate amino acid sequence. Hybridization at temperatures below the melting point (Tₘ) of the probe/conjugate complex will allow pairing to non-identical, but highly homologous sequences. For example, a hybridization at 60° C. of a probe that has a Tₘ of 70° C. will permit ˜10% mismatch. Washing at room temperature will allow the annealed probes to remain bound to target DNA sequences. Hybridization at temperatures (e.g., just below the predicted Tₘ of the probe/conjugate complex) will prevent mismatched DNA targets from being bound by the DNA probe. Washes at high temperature will further prevent imperfect probe/sequence binding. Exemplary hybridization conditions are as follows: hybridization overnight at 50° C. in APH solution [5×SSC (where 1×SSC is 150 mM NaCl, 15 mM sodium citrate, pH 7), 5× Denhardt's solution, 1% sodium dodecyl sulfate (SDS), 100 μg/ml single stranded DNA (salmon sperm DNA)] with 10 ng/ml probe, and washing twice at room temperature for ten minutes with a wash solution comprising 2×SSC and 0.1% SDS. Exemplary stringent hybridization conditions, useful for identifying interspecies prion counterpart sequences and intraspecies allelic variants, are as follows: hybridization overnight at 68° C. in APH solution with 10 ng/ml probe; washing once at room temperature for ten minutes in a wash solution comprising 2×SSC and 0.1% SDS; and washing twice for 15 minutes at 68° C. with a wash solution comprising 0.1×SSC and 0.1% SDS.

In another alternative embodiment, known prion sequences or other SCHAG amino acid sequences are modified, e.g., by addition, deletion, or substitution of individual amino acids; or by repeating or deleting motifs known or suspected of influencing fibril-forming propensity. To form novel prion sequences, modifications to increase the number of polar residues (glutamine, asparagine, sorine, tyrosine) are specifically contemplated, with modifications that increase glutamine and asparagine content being highly preferred. [See Depace et al., Cell, 93:1241-1252 (1998), incorporated herein by reference.] In a preferred embodiment, the alterations are effected by site directed mutagenesis or de novo synthesis of encoding polynucleotides, followed by expression of the encoding polynucleotides.

In yet another alternative embodiment, antibodies are generated against the prion forming domain of a prion or prion-like protein, using standard techniques. See, e.g., Harlow and Lane, Antibodies, A Laboratory Manual, Cold Spring Harbor Laboratory, Cold Spring Harbor, N.Y. (1988). The antibodies are used to probe a Western blot of proteins for interspecies counterparts of the protein, or other proteins that possess highly conserved prion epitopes. Candidate proteins are purified and partially sequenced. The amino acid sequence information is used to generate probes for obtaining an encoding DNA or cDNA from a genomic or cDNA library using standard techniques.

Sequences identified by the foregoing techniques can be further evaluated for certain features that appear to be conserved in prion-like proteins, such as a region of 50 to 150 amino acids near the protein's amino-terminus or carboxyl-terminus that is rich in glycine, glutamine, and asparagine, and possibly the polar residues serine and tyrosine, which region may contain several oligopeptide repeats and have a predicted high degree of flexibility (based on primary structure). In the case of Sup35, a highly charged domain separates the flexible N-terminal region having these properties from the functional C-terminal domain. Sequences possessing one or more of these features are ranked as preferred prion candidates for screening according to techniques described in the following section.

By way of example, the Genbank protein database (accessible via the worldwide web at www.ncbi.nlm.nih.gov) was screened using the Basic Local Alignment Search Tool (BLAST) program (version 1.4.9) using the standard (default) matrix and stringency parameters (BLOSUM62). The prion forming domains of Ure2 (Genbank Acc. No. M35268, SEQ ID NO: 4, amino acids 1-65) and Sup35 (Genbank Acc. No. M21129, SEQ ID NO: 2, amino acids 1-114) from S. cerevisiae were used as BLAST query sequences. Open reading frames (ORFs) from S. cerevisiae with high similarity scores [P(N) less than 4×10⁻⁶] resulting from the initial search included the following Genbank database entries:
(1) residues 53-97 from Accession No. Z73582 (SEQ ID NO: 22), an uncharacterized open reading from S. cerevisiae;
(2) residues 1030-1071 from PID No. e236901, in Accession No. Z71255 (SEQ ID NO: 23), an uncharacterized open reading from S. cerevisiae;
(3) residues 4-58 from locus ybm6, Accession No. P38216 (SEQ ID NO: 24), an uncharacterized open reading from S. cerevisiae;
(4) residues 251-380 from locus hrp1, Accession No. U35737 (SEQ ID NO: 25), an RNA binding and transport protein having homology to hnRNP1 in humans.
(5) residues 28-126 from locus npl3, Accession No. U33077 (SEQ ID NO: 26), an RNA binding and transport protein that functions genetically in the same pathway as Hrp1;
(6) residues 97-286 from locus mcm1, Accession No. X14187 (SEQ ID NO: 27), a DNA binding protein active in cell cycle regulation and mating-type specificity;
(7) residues 205-414 from locus nsr1, Accession No. P27476 (SEQ ID NO: 28), a protein that binds nuclear localization sequences and is active in mRNA processing;
(8) residues 153-405 from Accession No. P25367 (SEQ ID NO: 29), an uncharacterized open reading frame;
(9) residues 806-906 from Accession No. P40467 (SEQ ID NO: 30), an uncharacterized open reading frame;
(10) residues 605-677 from Accession No. S54522 (SEQ ID NO: 31), an uncharacterized open reading frame;
(11) residues 100-300 from locus yk76, Accession No. P36168 (SEQ ID NO: 32), an uncharacterized open reading frame;
(12) residues 1 to 250 from locus fps1, Accession No. S16712 (SEQ ID NO: 33), a membrane channel protein that controls passive efflux of glycerol;
(13) residues 334-388 from Accession No. p40002 (SEQ ID NO: 34), an uncharacterized open reading frame;
(14) residues 325-375 from locus mad1, Accession No. P40957 (SEQ ID NO: 35), an uncharacterized open reading frame; and
(15) residues 215-284 from locus kar1, Accession No. M15683 (SEQ ID NO: 36), an uncharacterized open reading frame.

The nuclear polyadenylated RNA-binding protein hrp1 (Genbank Accession No. U35737) is an especially promising prion candidate. It is the clear yeast homologue of a nematode protein previously cloned by cross-hybridization with the human PrP gene; it scored highly (p value 3.9 e-5) in a Genbank BLAST search for sequences having homology to the N-terminal domain of Sup35; and it contains a stretch of 130 amino acids at its C-terminus that is glyine- and asparagine-rich and contains repeat sequences similar to the oligomeric repeats in the N-terminal domain of Sup35; and is predicted by secondary structure programs to consist entirely of turns.

The sequence corresponding to residues 153-405 of SEQ ID NO: 29 comprises another promising prion candidate. This region is rich in glutamine and asparagine, and is part of a protein that is normally found in aggregates in yeast although it is not aggregated in some strains. When expressed as a fusion protein with green fluorescent protein, this sequence causes the GFP to aggregate. This aggregation is completely dependent upon Hsp104, much the same as Sup35 aggregation. When residues 153-405 of SEQ ID NO: 29 are substituted for the NM region of SUP35 and transformed into [psi−] yeast, the yeast exhibit a suppression phenotype analogous to [PSI⁺].

B. Screening Sequences to Confirm Prion-forming Capability.

Sequences identified according to methods set forth in Section A are screened to determine if the sequences represent/encode proteins having the ability to aggregate in a prion-like manner.

1. Aggregation Assay Using Fusion Proteins

In a preferred screening technique, a polynucleotide encoding the ORF of interest is amplified from DNA or RNA from a host cell using polymerase chain reaction, or is synthesized using the well-known universal genetic code and using an automated synthesizer, or is isolated from the host cell of origin. The polynucleotide is ligated in-frame with a polynucleotide encoding a marker sequence, such as green fluorescent protein or firefly luciferase, to create a chimeric gene. In a preferred embodiment, the polynucleotide is ligated in frame with a polynucleotide encoding a fusion protein such as a Bleomycin/luciferase fusion, which would permit both selection for drug-resistance and quantification of soluble and insoluble proteins by enzymatic assay. See, e.g., Elgersma et al., Genetics, 135: 731-740 (1993).

The chimeric gene is then inserted into an expression vector, preferably a high-copy vector and/or a vector with a constitutive or inducible promoter to permit high expression of the ORF-marker fusion protein in a suitable host, e.g., yeast. The expression construct is transformed or transfected into the host, and transformants are grown under conditions that promote expression of the fusion protein. Depending on the marker, the cells may be analyzed for marker protein activity, wherein absence of marker protein activity despite the presence of the marker protein is correlated with a likelihood that the ORF has aggregated, causing loss of the marker activity. Alternatively, host cells or host cell lysates are analyzed to determine if the fusion protein in some or all of the cells has aggregated into aggregates such as fibril-like structures characteristic of prions. The analysis is conducted using one or more standard techniques, including microscopic examination for fibril-like structures or for coalescence of marker protein activity; analysis for sensitivity or resistance to protease K; spectropolarimetric analysis for circular dichroism that is characteristic of amyloid proteins; and/or Congo Red dye binding.

A number of the candidates identified above were screened in this manner using a GFP fusion construct. To create the vector that was employed in these analyses, a copper inducible Cup1 promoter was amplified from a genomic library by standard polymerase chain reaction (PCR) methods using the primers 5′-GGGAATTCCCATTACCGACATTTGGGCGC-3′ (SEQ ID NO: 37) and 5′-GGGGATCCTGATTGATTGATTGATTGTAC-3′ (SEQ ID NO: 38), digested with the restriction enzymes EcoRI and BamHI, and ligated into the pRS316 vector that had digested with EcoRI and BamHI. The annealed vector, designated pRS316Cup1, was transformed into E. Coli strain AG-1, and transformants were selected using the ampicillin resistance marker of the vector. Correctly transformed bacteria were grown overnight to provide DNA for further vector construction.

Next, a sequence encoding superbright GFP (SEQ ID NOs: 39, 40) was inserted into the pRS316Cup1 vector. Superbright GFP was amplified from pPSGFP using the primers 5′-GACCGCGGATGGCTAGCAAAGGAGAAG-3′ (SEQ ID NO: 41) and 5′-CCTGAGCTCTCATTTGTATAGTTCATCC-3′ (SEQ ID NO: 42). The resultant PCR products were digested with SacI and SacII and inserted into PRS316Cup1 that also had been digested ed with SacI and SacII. This created a pRS316Cup1GFP plasmid into which a polynucleotide encoding a candidate open reading frame could be inserted for expression studies. In particular, it was contemplated that candidate open reading frames be amplified by PCR from genomic DNA or cDNA using primers engineered to contain BamHI and SacIl restriction sites, to permit rapid cloning into the BamHI and SacII sites of the derived PRS316Cup1GFP vector. For example, in the case of open reading frame (ORF) P25367 the following primers were used: 5′-GGAGGATCCATGGATACGGATAAGTTAATCTCAG-3′ (SEQ ID NO: 43, BamHI site underlined) and 5′-GGACCGCGGGTAGCGGTTCTGTTGAGAAAAGTTGCC-3′ (SEQ ID NO: 44, SacII site underlined). PCR products were digested with BamHI and SacII and inserted into the derived plasmid. This created a plasmid that can inducibly express a fusion of an open reading frame of interest fused to GFP. The sequence of pRS316-Cup1-p25367-GFP is set forth in SEQ ID NO: 45.

2. In Vitro Aggregation Assay Using Chaperone Protein

A polynucleotide encoding the ORF of interest is synthesized using the well-known universal genetic code and using an automated synthesizer, or is isolated from the host cell of origin, or is amplified using polymerase chain reaction from DNA or RNA from such a host cell. In a preferred embodiment, the polynucleotide further includes a sequence encoding a tag sequence, such as a polyhistidine tag, HA tag, or FLAG tag, to facilitate purification of the recombinant protein. The polynucleotide is inserted into an expression vector and expressed in a host cell compatible with the selected vector, and the resultant recombinant protein is purified.

Serial dilutions of the recombinant polypeptide (e.g., 100 mM, 10 mM, 1 mM, 0.1 mM, 0.01 mM final concentration) are mixed with 1 μg of a chaperone protein such as yeast Hsp104 protein [See Schirmer and Lindquist, Meth. Enzymol., 290: 430-444 (1998)] in a low salt buffer (e.g., 10 mM MES, pH 6.5, 10 mM MgSO₄) containing 5 mM ATP in a 25 μl reaction volume. As controls, reactions are performed in parallel using buffer alone or using Sup35 protein. Reactions are incubated at 37° C. for eight minutes, and the ATPase activity of the chaperone protein is measured by determining released phosphate., e.g., using Malachite Green [Lanzetta et al., Analyt. Biochem., 100: 95-97 (1979)]. In this assay, several fibril-aggregation proteins, including yeast Sup35, the yeast Sup35 N terminal domain, mammalian PrP protein, and β-amyloid (1-40) and (1-42) forms, were found to inhibit the ATPase activity of Hsp104; whereas control proteins (aldolase, BSA, apoferritin, and IgM) did not.

3. Assay Results

To determine if the proteins represented by the ORF's identified above in part A were aggregation prone, a hallmark of prions, polynucleotides encoding the specified residues of interest within the ORF's were amplified from S. cerevisiae genomic DNA via PCR and ligated in-frame to a sequence encoding superbright, as described above in section B.1.

These plasmids were transformed into the yeast strain 74D (a, his, met, leu, ura, ade). Transformant colonies were selected (ura+) and inoculated into liquid SD ura and grown to early log phase. Copper sulfate was added to the cultures (final concentration 50 μM copper) to induce protein expression. Cells were fixed after four hours of induction and intracellular GFP expression was visualized.

Examination of GFP fluorescence revealed that the sGFP tag had coalesced in transformants expressing six of the ORF's. This coalescence was similar to that observed with Sup35-GFP fusions in [PSI⁺] yeast and was considered to be indicative of an ORF having prion-like aggregate-forming ability. Two of the positive sequences represent uncharacterized open reading frames: Z73582 and ybm6. Four are known proteins: mcm1, fps1, p25367 and hrp1 as described above in section B.1. Aggregation of the MCM1-GFP fusion was relatively rare, and was not influenced by Hsp104 dosage in the cells. Of particular interest was the hrp1 construct, which aggregated into multiple cytoplasmic points in the transformed S. cerevisiae, and also in transformed C. elegans. Deletion of the Hsp104 gene was shown to eliminate the aggregation pattern of hrp1. Also of special interest was the aggregation pattern of the P25367 construct, because this aggregation was completely eliminated by overexpression of Hsp104.

The foregoing experiments demonstrate that searches with prion forming sequences will identify additional sequences with prion-like properties, which sequences can be used according to various aspects of the invention that are specifically exemplified herein with respect to Sup35 or URE2 sequences.

The ability of newly identified aggregating proteins to exist in both an aggregating and non-aggregating conformational state can be further examined, if desired, by studying aggregation phenomena in host cells expressing varying levels of the protein (a result achieved using an inducible promoter, for example), and in host cells having normal and over- or under-expressed chaperone protein levels. (The ability of Sup35 in yeast to enter a [PSI⁺] conformation depends on an appropriate intermediate level of the chaperone protein Hsp104; elimination of Hsp104 or over-expression of Hsp104 causes loss of [PSI⁺] and prevents de novo appearance of [PSI⁺]. See Chernoff et al., Science, 268: 880 (1995) and Patino et al., Science, 273: 622-626 (1996). Growth on a mildly denaturing media, as described elsewhere herein, provides another alternative assay.

The foregoing assays, chimeric constructs, and candidate SCHAG amino acid sequences are all intended as aspects of the invention.

### EXAMPLE 6

### Identification of Rnq1 as an Epigenetic Modifier of Protein Function in Yeast

The following experiments demonstrate that putative prions can be identified by searching for three attributes of the known yeast prion proteins: unusual amino-acid composition with a high concentration of the polar amino-acid residues glutamine and asparagine, constant expression levels through log and stationary phase growth, and a capacity to switch between distinct stable physical states (in this case, insoluble and soluble forms). One of the candidates isolated in this search, Rnq1, has both in vitro and in vivo characteristics of a prion. Rnq1, exists in distinct, heritable physical states, soluble and insoluble. The insoluble state is dominant and transmitted between cells through the cytoplasm. When the prion-like region of Rnq1 was substituted for the prion domain of Sup35, the protein determinant of the prion [PSI⁺], the phenotypic and epigenetic behavior of [PSI⁺] was fully recapitulated. These findings identify Rnq1 as a prion, demonstrate that prion domains are modular and transferable, and establish a paradigm for identifying and characterizing novel prions.

A. Identification of Prion Candidates

The characteristics of Sup35 and Ure2 suggested several criteria for identifying new prion candidates. Previous experiments have demonstrated that particular regions (residues 1-65 for Ure2 (Genbank Acc. No. M35268, SEQ ID NO: 4) and residues 1-123 for Sup35 (Genbank Acc. No. M21129, SEQ DI NO: 2)) are critical for prion formation by these proteins. Over-expression of these regions is sufficient to induce the prion phenotype de novo. Deletion of these regions has no effect upon the normal cellular function of the proteins but prevents them from entering the prion state. These critical prion-determining domains have an unusually high concentration of the polar residues glutamine and asparagine and are predicted to have very little secondary structure. The domains are located at the ends of proteins that have an otherwise ordinary amino acid composition. We hypothesized that by searching for open reading frames with these characteristics we might find new prion proteins.

A BLAST search (1.4.9MP version) of the NCBI database of non-redundant coding sequences was performed using the prion-determining domains of Ure2 and Sup35 (residues 1-65 of SEQ ID NO: 4 and residues 1-123 of SEQ ID NO: 2, respectively) as the query sequence with the following parameters: V=100, B=50, H=0, S=90, and P=4. This search revealed approximately twenty open reading frames that had prion-like domains appended to polypeptides with an otherwise normal amino acid composition. To restrict the number of likely candidates, we took advantage of recent global descriptions of mRNA expression patterns. In examining this data we noted that Sup35 and Ure2 are expressed at nearly constant levels as cells transit from the log to the stationary phase of growth. Large fluctuations in expression would be inconsistent with the stability of both their heritable prion and non-prion states. The open reading frames from the BLAST search whose expression varies by less than two-fold in the log phase transition were selected for further analysis. They were fused to the coding sequence of green fluorescent protein (GFP) using PCR and expressed in the yeast strain 74D-694 (ade1-14, trp1-289, his3-200, ura3-52, leu2-3, lys2). Three of the proteins, RNQ1 (Genbank Acc. No. NP009902, SEQ ID NO: 50), YBR016w (Genbank Acc. No. NP009572, SEQ ID NO: 51), and HRP1 (Genbank Acc. No. NP014518, SEQ ID NO: 52), showed coalescence of GFP, as previously described for Sup35.

B. Rnq1 Exists in Distinct States Controllable by Hsp104

We next asked if expression of the fusion protein in a strain that lacked the chaperone Hsp104 eliminated the coalescence of GFP, as it does for Sup35-GFP fusions. This is not a necessary criterion for prion proteins (an interaction with Hsp104 has not been demonstrated for [URE3]) but interaction with the chaperone provides a useful tool for further analysis. In wild-type yeast, fluorescence from the Rnq1-GFP fusion was found in one or more small, intense, cytoplasmic foci. When the fusion protein was expressed in the isogenic hsp104 strain, fluorescence was diffuse. The C-terminal end of Rnq1 (amino acids 153-405 of SEQ ID NO: 50) contained the region rich in glutamine and asparagine residues. Fusion of this region alone to GFP gave an identical result to that seen with the full length Rnq1-GFP fusion. Since the effect of HSP104 deletion upon the coalescence of the Rnq1 fusion was the most dramatic, it was chosen for further analysis.

Differential centrifugation was employed to determine if the coalescence observed with Rnq1-GFP fusion proteins reflected the behavior of the endogenous Rnq1 protein. Log phase yeast were lysed using a bead beater (Biospec) into 75mM Tris-Cl (pH7), 200 mM NaCl, 0.5 mM EDTA, 2.5% glycerol, 0.25 mM EDTA, 0.25% Na-deoxycholate, supplemented with protease inhibitors (Boehringer-Mannheim). Lysates were cleared of crude cellular debris by a 15 second 6000 RPM spin in a microcentrifuge (Eppendorf). Non-denatured total cellular lysates were fractionated by high-speed centrifugation into supernatant and pellet fractions using a TLA-100 rotor on an Optima TL ultracentrifuge (Beckman) at 280,000×g (85,000 RPM) for 30 minutes. Protein fractions were resolved by 10% SDS-PAGE and immunoblotted with an α-Rnq1 antibody. Rnq1 remained in the supernatant of a hsp104 strain, but pelleted in the wild-type. Thus, the GFP coalescence is not an artifact of the fusion; the Rnq1 protein itself is sequestered into an insoluble aggregate in an Hsp104-dependent fashion. We also examined the solubility of Rnq1 in several unrelated yeast strains. In four (S288c, YJM436, SK1 and W303) the protein fractionated in the pellet, in two (YJM128, YJM309) it partitioned between the pellet and supernatant fractions, and in two others (33G, 10B-H49) the protein was chiefly recovered in the supernatant fraction. Thus, Rnq1 naturally exists in distinct physical states in different strains.

C. The Insoluble State of Rnq1 is Transmitted by Cytoduction

The heritability of the known yeast prions is based upon the ability of protein in the prion state to influence other protein of the same sequence to adopt the same state. Because the protein is passed from cell to cell through the cytoplasm, the conformational conversion is heritable, dominant in crosses, and segregates in a non-Mendelian manner. To determine if the insoluble state of Rnq1 is transmissible in this way, we used cytoduction, a well-established tool for the analysis of the [PSI⁺] and [URE3] prion. The karyogamy deficient (kar1-1) strain 10B-H49(ade2-1, lys1-1, his3-11,15, leu2-3,112, kar1-1, ura3::KANR) can undergo normal conjugation between a and cells but is unable to fuse its nucleus with its mating partner. Cytoplasmic proteins and organelles are mixed in fused cells, but the haploid progeny that bud from them contain nuclear information from only one of the two parents.

10B-H49 shows diffuse expression of Rnq1-GFP, and served as the recipient for the transfer of insoluble Rnq1 from W303 (Mata, his3-11,15, leu2-3,112, trp1-1, ura3-1, ade2-1), the donor. After cytoduction, colonies derived from haploid cells that contained the 10B-H49 nuclear genome but had undergone cytoplasmic mixing, as demonstrated by mitochondrial transfer, were selected. Cytoductants were selected after overnight mating on defined media lacking tryptophan that had glycerol as the sole carbon source. All showed single or multiple cytoplasmic aggregates of Rnq1 -GFP—a pattern indistinguishable from that of the W303 parent. Furthermore, density-based centrifugation of protein extracts, performed as above, indicated that cytoduction caused the endogenous Rnq1 protein of the 10B-H49 strain to shift from the soluble to the insoluble fraction. Thus exposure of 10B-H49 cells to the cytoplasm of W303 is sufficient to cause a heritable change in the physical state of Rnq1. Because RNQ1 is a nuclear gene (not transmitted during cytoduction) the protein's insoluble state is not due to polymorphisms in its amino acid sequence, nor to any other trait carried by the W303 genome. Rather, like the Sup35 and Ure2 prions, its altered conformational state is “infectious”, transmissible from one protein to another.

D. Purified Rnq1 Forms Fibers and Shows Seeded Polymerization

Both Sup35 and Ure2 have the capacity to form highly ordered amyloid fibers in vitro, as analyzed by the binding of amyloid specific dyes and by electron microscopy. To examine conformational transitions of Rnq1 in vitro, the protein was expressed in E. coli and studied as a purified protein. Rnq1 was cloned into pPROEX-HTh (GibcoBRL). The primers 5′-GGA GGA TCC ATG GAT ACG GAT AAG TTA ATC TCAG-3′ (SEQ ID NO: 53) and 5′-CC AAG CTT TCA GTA GCG GTT CTG TTG AGA AAA GTTG-3′ (SEQ ID NO: 54) were used for PCR in a solution containing 10 mM Tris (pH8.3), 50 mM KCl, 2.5 mM MgCl₂,2 mM dNTPs, 1 μM of each primer and 2 U of Taq polymerase; and using genomic 74D DNA as template under the following conditions: incubation at 94° C. for 2 min, followed by 29 cycles of 94° C. for 30 sec, 50° C. for 30 sec, and 72° C. for 90 sec, followed by a final incubation at 72° C. for 10 minutes. The PCR product was then digested and ligated into the BamHI and HindIII sites of pPROEX-HTh (GibcoBRL). The plasmid was electroporated into BL21-DE3 laciq cells. Transformed bacterial cultures were induced at OD₆₀₀=1 with 1 mM IPTG for four hours at 30C. The cells were lysed in 8M urea (Rnq1 was purified under denaturing conditions (8M urea) because it had a tendency to form gels during purification in the absence of denaturant), 20 mM Tris-Cl pH8. Protein was purified over a Ni-NTA column (Qiagen) followed by Q-sepharose (Pharmacia). The (His)₆-tag from the vector was cleaved under native conditions (150 mM NaCl, 5 mM KPi) using TEV protease followed by passage of the protease product over a Ni-NTA column to remove uncleaved protein. Protein was methanol precipitated prior to use. Recombinant protein was resuspended in 4M urea, 150 mM NaCl, 5 mM KPi, pH 7.4 at a concentration of 10 μM. Seeded samples were created by sonication of 1/50 volume of a 10 μM solution of pre-formed fibers verified by electron microscopy. The protein samples were incubated at room temperature on a wheel rotating at 60 r.p.m.

To determine if Rnq1 forms amyloids we used Thioflavin T fluorescence. This dye exhibits an increase in fluorescence and a red-shift in the max of emission upon binding to multimeric fibrillar β-sheet structures characteristic of many amyloids, including transthyretin, insulin, β-2 microglobulin and Sup35. Fluorimeter samples were prepared as 3.3 μM Rnq1, 50 μM Thioflavin T in buffer. Samples were analyzed on a Jasco FP750 with the following settings: _{exc}=409 nm, ₑₘᵢ=484 nm, bandwidth 10 nm. The acquisition of Thioflavin T binding was sigmoidal (lag phase˜six) suggesting a self-seeded process of protein assembly. The addition of 2% preformed fibers to fresh solutions of Rnq1 reduced the lag time—from 6.40.2 hrs to 4.30.2 hrs (n=4).

The formation of higher ordered structures was confirmed by transmission electron microscopy. For electron microscopy analysis, 5 μl of a 10 μM protein solution was placed on a 400 mesh carbon coated EM grid (Ted Pella, Cat. 01822), and allowed to adsorb for 1 minute. The sample was negatively stained with 200 μl of 2% aqueous uranyl acetate, and wicked dry. Samples were observed in a Philips CM120 transmission electron microscope operating at 120 kV in low dose mode. Micrographs were recorded at a magnification of 45,000 on Kodak SO-163 film. The protein formed fibers with a diameter of 11.3 1.4 nm. This figure is comparable to the reported range for Ure2 (˜20 nm) and Sup35 (˜17 nm) fibers. The fibers appeared to be branching and the termini were unremarkable. The appearance of the fibers was coincident with the onset of rapid increases in Thioflavin T fluorescence.

E. Rnq1 Disruption

[URE3] and [PSI⁺] produce phenotypes that mimic loss-of-function mutations in their protein determinants. To determine the loss of function phenotype of Rnq1, the entire ORF was deleted by homologous recombination in a diploid 74D-694 strain using a kanamycin resistance gene. Strains deleted of the Rnq1 open reading frame were created using the long flanking homology PCR method. Primers 5′-GGT GTC TTG GCC AAT TGC CC-3′ (SEQ ID NO: 55) and 5′-GTC GAC CTG CAG CGT ACG CAT TTC AGA TCT TTG CTA TAC-3′ (SEQ ID NO: 56) or 5′-CGA GCT CGA ATT CAT CGA TTG ATT CAG TTC GCC TTC TATC-3′ (SEQ ID NO: 57) and 5′-CTG TTT TGA AAG GGT CCA CATG-3′ (SEQ ID NO: 58) were used to amplify genomic DNA. These PCR products were used as primers for a second round of PCR on plasmid pFA6a,which is described in Wach et al., Yeast 13:1065-75 (1994), digested with NotI. The product of the second PCR round was used to transform log-phase yeast cultures. Transformants were selected on YPD containing 200 mg/mL G418 (GibcoBRL). Upon sporulation each tetrad produced four viable colonies, two of which contained the Rnq1 disruption, confirmed by immunoblotting total cellular proteins with an −Rnq1 antibody and PCR analysis of the genomic region. The rnq1 strain had a growth rate comparable to that of wild-type cells on a variety of carbon and nitrogen sources and was competent for mating and sporulation. The strain grew similarly to the wild-type in media with high and low osmolarity, and in assays testing sensitivity to various metals (cadmium, cobalt, copper).

F. Fusion of Rnq1 (153-405) to Sup35 (124-685)—Nonsense Suppression Phenotype

The lack of an obvious loss-of-function phenotype was not unexpected, as the two known yeast prions, [URE3] and [PSI⁺] only exhibit phenotypes under unusual selective conditions. However, the absence of a phenotype presented difficulties in determining whether Rnq1 could direct the epigenetic inheritance of a trait. To determine if the prion-like domain of Rnq1 could produce an epigenetic loss-of-function phenotype we asked if it could replace the prion-determining domain of Sup35. When the wild-type Sup35 translation termination factor enters the prion state the loss-of-function phenotype it produces is nonsense suppression—the readthrough of stop codons. This phenotype can be conveniently assayed in the strain 74D-694 because it contains a UGA stop codon in the ADE1 gene. In [psi⁻] 74D-694 cells, ribosomes efficiently terminate translation at this codon. Cells are therefore unable to grow on media lacking adenine (SD-ade), and colonies appear red on rich media due to the accumulation of a pigmented by-product. In [PSI⁺] strains, sufficient readthrough occurs to support growth on SD-ade and prevent accumulation of the pigment on rich media.

The coding region for amino acid residues 153-405 of Rnq1 (amino acid residues 153-405 of SEQ ID NO: 50) was substituted for 1-123 of Sup35 and the resulting fusion gene, RMC, was inserted into the genome in place of the endogenous SUP35 gene. RNQ1, SUP35 and its promoter were cloned by amplification of 74D-694 genomic DNA. The RNQ1 open reading frame was cloned using 5′-GGA GGA TCC ATG GAT ACG GAT AAG TTA ATC TCAG-3′ (SEQ ID NO: 59) and (A) 5′-GGA CCG CGG GTA GCG GTT CTG TTG AGA AAA GTT GCC-3′ (SEQ ID NO: 60). RNQ1 (153-405) was cloned using 5′-GA GGA TCC ATG CCT GAT GAT GAG GAA GAA GAC GAGG-3′ (SEQ ID NO: 61) and (A). The SUP35 promoter was cloned using 5′-CG GAA TTC CTC GAG AAG ATA TCC ATC-3′ (SEQ ID NO: 62) and 5′-G GGA TCC TGT TGC TAG TGG GCA GA-3′(SEQ ID NO: 63 ). SUP35 (124-685) was cloned using 5′-GTA CCG CGG ATG TCT TTG AAC GAC TTT CAA AAGC-3′ (SEQ ID NO: 64) and 5′-GTG GAG CTC TTA CTC GGC AAT TTT AAC AAT TTT AC-3′ (SEQ ID NO: 65) by PCR using the conditions described above in section D.

The RMC gene replacement was performed as described in Rothstein, 1991. To create the plasmid for pop-in/pop-out replacement in pRS306 (available from ATCC), the SUP35 promoter was ligated into the EcoRI-BamHI site, RNQ1 (153-405) was ligated into the BamHI-SacII site, and SUP35 (124-685) was ligated into the SacII-SacI site. To create the disrupting fragment, this plasmid was linearized with MluI and transformed. Pop-outs were selected on 5-FOA (Diagnostic Chemicals Ltd.) and verified by PCR. The resulting strain, RMC, had a growth rate similar to that of wild-type cells on YPD, although the accumulation of red pigment was not as intense as seen in [psi−] strains. RMC strains showed no growth on SD-ade even after 2 weeks of incubation). Thus, the protein encoded by the RMC gene (Rmc) fulfilled the essential translational termination function of Sup35.

At a low frequency, RMC variants appeared that were white on rich media and grew on SD-ade even more robustly than [PSI⁺] cells did. The frequency at which these variants appeared (˜10⁻⁴ ) was far greater than expected for reversion of the UGA stop codon mutation in ade1-14, and subsequent analysis demonstrated that the allele had not reverted. The suppressor phenotype of these variants was comparable in stability to that of [PSI⁺]. Because Sup35 proteins that lack residues 1-123 are incapable of making such conversions, these observations suggest that the Rnq1 prion-like domain can direct a prion conversion in the Rmc fusion protein.

Transient over-expression of Sup35 can produce new [PSI⁺] elements, because higher protein concentrations make it more likely that a prion conformation will be achieved. To test whether over-expression of Rmc can produce heritable suppressing variants, the original, non-suppressing RMC strain was transformed with an expression plasmid for RMC. These transformnants showed a greatly elevated frequency of conversion to the suppressor state compared to control strains carrying the plasmid alone. Once a prion conformation is achieved it should be self-perpetuating and normal expression should then be sufficient for maintenance. When the RMC expression plasmid was lost all strains retained the suppressor phenotype. Thus, transient over-expression of Rmc produced a heritable change in the fidelity of translation termination.

G. Non-Mendelian Segregation of Rmc-based Suppression Phenotype

To examine the genetic behavior of the suppressor phenotype in RMC strains, an isogenic mating partner was created from a non-suppressing a RMC strain. When this strain was crossed to the original, non-suppressing, RMC strain, neither the diploids nor their haploid meiotic progeny exhibited the suppressor phenotype. However, when this strain was mated to RMC suppressor strains, the resulting diploids all displayed the suppressor phenotype, demonstrating that suppression is dominant. In fourteen tetrads dissected from two different diploids of this cross, all four haploid progeny showed inheritance of the suppression phenotype, instead of the 2:2 segregation expected for a phenotype encoded in the nuclear genome. Following convention, we henceforth refer to the dominant, non-Mendelian suppressor phenotype as [RPS⁺] (for Rnq1 [PSI⁺]-like Suppression) and the non-suppressed phenotype as [rps⁻].

To determine if the dominant, non-Mendelian [RPS⁺] phenotype arises from the ability of Rmc protein to form a prion, we tested it for two additional unusual genetic behaviors that are not expected for other non-Mendelian genetic elements, such as viruses or mitochondrial genomes. First, it should become recessive and Mendelian in crosses to strains carrying a wild-type Sup35 allele. This is because Sup35 lacks the Rnq1 sequences that would allow it to be incorporated into an [RPS⁺] prion. Wild-type Sup35, therefore, should cover the impaired translation-termination phenotype associated with the [RPS⁺] prion. However, even when this phenotype has disappeared, Rmc protein in the prion state should still convert new Rmc protein to the same state. Therefore, in haploid meiotic progeny of this diploid, the phenotype will reappear in segregants carrying the RMC gene, but not in segregants carrying the SUP35 gene (2:2 segregation).

Indeed, diploids of a cross between an [RPS⁺] strain and an isogenic strain with a wild-type SUP35 gene did not exhibit a suppressor phenotype. Upon sporulation, suppression reappeared in only two of the four progeny. By PCR genotyping, these strains had the RMC gene at the SUP35 locus. Thus the [RPS⁺] factor had been preserved in the diploid, even though the phenotype had become cryptic.

Second, maintenance of [RPS⁺] should depend upon continued expression of the Rmc protein. Although [RPS⁺] is maintained in a cryptic state in diploids with a wild-type Sup35 gene, it should not be maintained in their haploid progeny whose only source of translational termination factor is wild-type Sup35. To determine if these progeny harbored the [RPS⁺] element in a cryptic state, they were mated to an [rps⁻] RMC strain whose protein would be converted if [RPS⁺] were still present. When this diploid was sporulated, none of the progeny exhibited the suppressor phenotype. Thus, the [RPS⁺] element was not maintained in a cryptic state unless the Rmc protein was present.

H. Curing of [RPS⁺]

One of the hallmarks of yeast prions is that cells can be readily and reversibly cured of them. [PSI⁺] is curable by several means, including growth on media containing low concentrations of the protein denaturant guanidine hydrochloride and transient over-expression or deletion of the protein remodeling factor HSP104.

Strains carrying [RPS⁺] were passaged on medium containing 2.5 mM guanidine hydrochloride (GdnHCl) (Fluka) and then plated to YPD and to SD-ade to assay the suppressor phenotype. Cells passaged on GdnHCl no longer displayed the [RPS⁺] phenotype, while cells not treated with GdnHCl retained it. [RPS⁺] was also lost when the HSP104 gene was deleted by homologous recombination, performed using the same strategy as described above in section E, or when HSP104 was over expressed from a multicopy plasmid using the constitutive GPD promoter. Cells that had been cured of [RPS⁺] by over-expression of HSP104 were passaged on YPD medium to isolate strains that had lost the over-expression plasmid. These strains remained [rps⁻]. Thus transient over-expression of HSP104 is sufficient to heritably cure cells of [RPS⁺].

Finally, we asked if Hsp104-mediated curing was reversible. Cells cured by over-expression of HSP104 were re-transformed with a plasmid bearing a single copy of RMC. To create the single-copy RMC plasmid in pRS316 (available from ATCC) the Clal-SacI fragment (includes promoter and RMC) from the plasmid used above for the RMC gene replacement was ligated into the ClaI-SacI site. Transformants were then plated onto SD-ade to assess the rate at which they converted to the [RPS⁺] suppressor phenotype. [RPS⁺] was regained at a rate comparable to that seen in the parental RMC strain, indicating that the transient over-expression of HSP104 caused no permanent alteration in susceptibility to [RPS⁺] conversion.

I. Effect of Endogenous Rnq1 upon [RPS⁺]

To determine if [RPS⁺] can act as an independent genetic element, the gene encoding the endogenous Rnq1 protein was deleted in strains carrying the RMC replacement of SUP35 using methods described above. The deletion had no effect upon the maintenance of the [RPS⁺] suppression phenotype. Growth on SD-ade was equally robust in [RPS⁺] and [RPS⁺] rnq1 strains. This indicates that Rmc can behave as an independent prion and is not dependent upon pre-existing Rnq1 in an insoluble state.

J. Physical State of the Rmc Protein in [RPS⁺] and [rps⁻] Strains

Finally, we examined the localization of the Rmc fusion protein in the [RPS⁺] and [rps⁻] strains. Both strains were transformed with inducible plasmids that provided Rnq1(153-405)-GFP expression that were constructed as described above in section A. Strains that lacked the endogenous Rnq1 gene were used to prevent the GFP marker from localizing to the endogenous Rnq1 aggregate. Short-term expression of the GFP-fusion protein prevented the formation of new [RPS⁺] elements in the [rps⁻] strain.

Two distinct patterns of Rmc protein localization were revealed by this assay and these correlated with the phenotypic differences between [RPS⁺] and [rps⁻] strains. In the non-suppressing [rps⁻] strains, the Rnq1(153-405)-GFP label was diffuse. In the suppressing [RPS⁺] strains, fluorescence was punctate, and was excluded from the nucleus. This punctate pattern was different from that observed with the endogenous Rnq1 aggregates, as Rmc aggregates are numerous and very small.

Collectively, the foregoing experiments demonstrate that Rnq1, which was identified based on sequence analysis, exhibits prion-like behavior in numerous in vitro and in vivo assays. The search method used here shows that putative prions can be identified by a directed prion search rather than by the study of a pre-existing phenotype. In addition, this method will be applicable to the identification of prion proteins in many other organisms. Our demonstration that a new prion protein domain can substitute for that of another well-characterized prion, reproducing its phenotypic characteristics and epigenetic mode of inheritance, also provides a crucial tool in the analysis of uncharacterized candidates.

We have shown that Rnq1 exists in distinct physical states—soluble and insoluble—in unrelated yeast strains. The insoluble state can be transmitted through cytoduction, and once transmitted is stably inherited. When the N-terminal prion-determining region of SUP35 was replaced with the C-terminal domain of RNQ1, the hybrid Rmc protein provided translation termination activity, mimicking the phenotype of [psi⁻] strains. At a low spontaneous frequency, the strain acquired a stable, heritable suppressor phenotype, [RPS⁺], which mimicked the phenotype of [PSI⁺] strains. Suppression was dominant and segregated to meiotic progeny in non-Mendelian ratios. The possibility that this phenotype is caused by an epigenetic factor unrelated to the fusion protein was ruled out by genetic crosses showing that the phenotype is not expressed and can not be transmitted in strains that do not produce the fusion protein. The relationship of the suppression phenotype to protein conformation was further demonstrated by fluorescence localization of the hybrid protein in isogenic [RPS⁺] and [rps⁻] strains. In [RPS⁺] strains, most of the protein is sequestered into small foci and is presumably inhibited in its function in translational termination. Transient over-expression of Rmc greatly increased the frequency of conversion to [RPS⁺].

It is highly unusual for over-expression of a protein to cause a loss-of-function phenotype. It is even more unusual for phenotypes produced by over-expression to be stable after over-expression has ceased. Yet these properties are shared by the two yeast prion determinants and, to our knowledge, have been uniquely shared by them until now. They are believed to derive from stabilization of an otherwise unstable protein conformation by protein-protein interactions. Proteins in the altered form then have the capacity to recruit new proteins of the same type to the same form. The phenotype associated with this change is, therefore, stably inherited from generation to generation and transferred to mating partners in crosses.

The ability of amino acid residues 153-405 of Rnq1(SEQ ID NO: 50) to substitute for the N-terminal domain of Sup35 and recapitulate its prion behavior was by no means predictable. The C-terminal region of Rnq1 (residues 153-405) and the N-terminal region of Sup35 have no primary amino-acid sequence homology—only a similar enrichment in polar amino acids. Reconstituting the epigenetic behavior of a prion requires that the Rmc fusion protein achieve an unusual balance between solubility and aggregation. If the fusion protein is too likely to aggregate, the inactive state will be ubiquitous; if it is too likely to remain soluble, the inactive state will not be stable. To recapitulate the epigenetic behavior of [PSI⁺] the fusion protein must be able to switch from one state to the other and maintain either the inactive or the active state in a manner that is self perpetuating and highly stable from generation to generation. Even minor variations in the sequence of the N-terminal region of Sup35, including several single amino-acid substitutions and small deletions, can prevent maintenance of the inactive state. And a small internal duplication destabilizes maintenance of the active state. Therefore, the ability of the Rnq1 domain to substitute for the prion domain of Sup35 and to fully recapitulate its epigenetic behavior provides a rigorous test for its capacity to act as a prion and suggests that it has been honed through evolution to serve this function.

The fusion of prion-determining regions with different functional proteins could be used to create a variety of recombinant proteins whose functions can be switched on or off in a heritable manner, both by nature and by experimental design. The two regions that constitute a prion, a functional domain and an epigenetic modifier of function, are modular and transferable.

### EXAMPLE 8

### High-Throughput Assay to Identify Novel Prion-like Amyloidogenic Sequences

The procedures described in Example 5 are particularly useful for identifying candidate prion-like sequences based on sequence characteristics and for screening these candidate sequences for useful prion-like properties. The following modification of those procedures provides a high-throughput genetic screen that is particularly useful for identifying sequences having prion-like properties from any set of clones, including a set of uncharacterized clones, such as cDNA or genomic libraries.

A library of short DNA fragments, such as genomic DNA fragments or cDNAs, is cloned in front of a sequence encoding the C-terminal domain of yeast Sup35 to create a library of CSup35 chimeric constructs of the formula 5′-X-CSup35-3′, wherein X is the candidate DNA fragment. Optionally, the 3′ end of the construct encodes both the M and C domains of Sup35. This library is transformed into a [psi⁻] strain of yeast that carries Sup35 as a Ura+ plasmid (with its chromosomal Sup35 deleted). Transformants are plated onto FOA-containing medium, which will cure the Ura+ plasmid so that the only functioning copy of Sup35 will be a fusion construct from the chimeric library.

Viable transformants are transferred to a selective media to screen for transformants which can suppress nonsense codons in a [PSI⁺]-like manner. For example, if the host cell is a yeast strain carrying a nonsense mutation in the ADE1 gene, the transformants are screened for cells that are viable on a SD-ADE media. Cells that can survive via suppression of nonsense codons are selected for further analysis (e.g., as described in preceding Examples), under the assumption that the library chimera has altered the function of Sup35. By using prion-specific tests such as histological examination for protein aggregates, curing, and Hsp104-dosage alteration, true aggregation-directing protein domains will be identified from original library of DNA constructs. The constructs which display prion-like properties can be used as described herein. Also, such constructs can be isolated and sequenced and used to identify and study the complete genes from which they were derived, to see if the original gene/protein possesses prion properties in its native host. The foregoing assay also is useful for rapidly identifying fragments and variants of known prion-like proteins (NMSup35, NUre2, PrP, and so on) that retain prion-like properties. The assay, as well as chimeric constructs of the formula 5′-X-CSup35-3′ and expression vectors containing such constructs, are considered additional aspects of the present invention.

### EXAMPLE 9

### Fiber Assembly Mechanism of the Prion-determining Region (NM) of Yeast Sup35p

The investigation of specific protein aggregation is gaining an increasing role in conjunction with increasing numbers of human diseases characterized by altered protein structures, including prion-based encephalopathies, noninfectious neurodegenerative diseases, and systemic amyloidoses. Amyloid protein aggregates are β-sheet rich structures that form fibers in vitro and bind dyes such as CongoRed and ThioflavinT. Strikingly, most amyloids can promote the propagation of their own altered conformations, which is thought to be the basis of protein-mediated infectivity in prion diseases. This feature of protein self-propagation in amyloids may also be critical to disease progression in noninfectious amyloid diseases such as Alzheimer's or Parkinson's disease. A powerful system to study the molecular mechanism of amyloid propagation and specificity is the prion-like phenomenon [PSI⁺] of Saccharomyces cerevisiae. Formation of higher ordered Sup35p complexes and the propagation of [PSI⁺] is caused by NM region of Sup35p. In vitro, both full-length Sup35p and NM form amyloid fibers with NM dictating the formation of the fiber axis while the C-terminal region of Sup35p is thought to be located on the periphery of the fibers. Detailed analysis by circular dichroism showed that NM adopts a mainly random coil structure in solution before it changes slowly to a structure that is β-sheet-rich. This conformational conversion was shown to occur simultaneously to the formation of amyloid fibrils.

In general, amyloid polymerization is considered to be a two-stage process initiated by the formation of a small nucleating seed or protofibril. Seed formation is thought to be oligomerization of soluble protein accompanied by a transition from a predominantly random coil to an amyloidogenic β-sheet conformation. Subsequent to nucleation, the seeds assemble with soluble protein to form the observed amyloid fibrils. The mechanisms for nucleation and fiber assembly are not well understood.

Strikingly, the secondary structure of all proteins that form amyloid fibrils under physiological conditions is partially random coil in aqueous solutions. Such structure is usually significant for partially unfolded protein as found in folding intermediates. It is possible that this unique “high-energy” structure in solution is the driving force for fiber assembly of such proteins. Thereby, the fibrous aggregates might present the lowest energy conformer of these proteins. As a consequence, interference with their structural state in solution should influence their fiber assembly ability. This has been shown for Alzheimer's β-amyloid peptide, islet amyloid polypeptide, and the artificial peptide DAR16-IV, where changes in the secondary structure dramatically altered the fiber assembly process.

The following experiments were performed to examine and characterize the folding and association pathway of soluble NM by starting with chemically denatured protein. Similar results were obtained with proteins isolated under non-denaturing conditions. These studies were facilitated by use of labeled cysteine-substituted NM mutants. A better understanding of the mechanisms of fiber assembly will facilitate manipulations of fiber growth under various conditions.

A. Materials and Methods

Bacterial Strains and Culture

Using pEMBL-Sup35p (an E. coli plasmid containing the Sup35 protein) as template, DNA encoding NM was amplified by PCR with various linkers for subcloning. For recombinant NM expression, the PCR products were subcloned as NdeI-BamHI fragments into pJC25. For GST-NM fusions, the PCR products were subcloned as BamHI-EcoRI fragments into pGEX-2T (Pharmacia). For site-directed mutagenesis the protocol by Howorka and Bayley, Biotechniques, 25:764-766 (1998), was used for a high throughput cysteine scanning mutagenesis. A non-mutagenic primer pair for the β-lactamase gene and a mutagenic primer pair for each respective mutant were employed. In addition to generating a unique NsiI site, we used SphI and NspI sites, which allows introduction of a cysteine codon in front of methionine and isoleucine or after alanine and threonine codons, to increase the number of mutants in our cysteine screen. The fidelity of each construct was confirmed by Sanger sequencing. Protein was expressed in E. coli BL21 [DE3] after inducing with 1 mM IPTG (OD₆₀₀ nm of 0.6) at 25° C. for 3 hours.

Yeast Strains and Culture

Using pJLI-Sup35pC-Sup35p as a template, DNA encoding each of the respective NM^{cys} was amplified by PCR with two EcoRI sites for subdloning. To investigate the propagation and maintenance of [PSI⁺] by each NM^{cys} used, integrative constructs, constructed using the standard pRS series of vectors (available from ATCC), were digested with XbaI and transformed into 74-D694 [PSI⁺] and [psi] strains. Transformants were selected on uracil-deficient (SD-Ura) medium and confirmed by genomic PCR followed by digestion with AatII, which cleaves the HA-tag between NM^{cys} and Sup35pC. Recombinant excision events were selected on medium containing 5-fluoro-orotic acid. Only cells that have lost remaining integrative plasmids are able to grow on medium containing 5-fluoro-orotic acid. Again, replacements were confirmed by PCR followed by digestion with AatII as described above.

Protein Purification

NM and each NM^{CYS} were purified after recombinant expression in E. coli by chromatography using Q-Sepharose (Pharmacia), hydroxyapatite (BioRad), and Poros HQ (Boehringer Mannheim) as a final step. All purification steps for NM or NM^{CYS} were performed in the presence of 8M urea. GST-NM was purified by chromatography using Glutathione-Sepharose (Boehringer Manheim), Poros HQ (Boehringer Mannheim), and S-Sepharose (Pharmacia) as a final step. All purification steps for GST-NM were performed in the presence of 50 mM Arginine-HCl. Protein concentrations were determined using the calculated extinction coefficient of 0.90 (NM, NM^{CYS}) or 1.23 (GST-NM) for a 1 mg/ml solution in a lcm cuvette at 280 nm.

Secondary Structure Prediction

Secondary structure of NM was predicted by using two independent prediction methods, GOR IV and Hierarchical Neural Network. Both methods were provided by Pole Bio-Infornatique Lyonnais.

Secondary Structure Analysis

CD spectra were obtained using a Jasco 715 spectropolarimeter equipped with a temperature control unit. All UV spectra were taken with a 0.1 cm pathlength quartz cuvette (Hellma) in 5 mM potassium phosphate (pH 7.4), 150 mM NaCl and respective additives such as osmolytes in certain experiments. Protein concentration varied from 0.5 μM to 65 μM. Folding of chemically denatured NM or NM^{CYS} was monitored at 222 nm in time course experiments by diluting protein out of 8M Gdm*Cl (Guanidinium Hcl; final concentration 50 mM) in the respective phosphate buffer. Thermal transition of NM or NM^{CYS} was performed with a heating/cooling increment of 0.5° C./min. Spectra were recorded between 200 nm and 250 nm (2 accumulations). In a separate measurement, time courses were recorded for 30 sec at single wavelengths (208 nm and 222 nm) for each temperature and the mean value of each time course was determined. Temperature jump experiments were performed by incubating the sample in a water bath with the respective starting temperature for 30 min. The cuvette was transferred to the spectropolarimeter already set to the final temperature and time courses were taken with a constant wavelength of 222 nm. Settings for wavelength scans: bandwidth, 5 nm; response time, 0.25 sec; speed, 20 nm/min; accumulations, 4. All spectra were buffer-corrected.

Fluorescent Labeling of NM^{CYS}

The thiol-reactive fluorescent labels acrylodan and IANBD amide (Molecular Probes) were incubated with NM^{cys} for 2 hours at 25° C. according to the manufacturer's protocol. Remaining free label was removed by size exclusion chromatography using D-Salt Excellulose desalting columns (Pierce). The labeling efficiencies were determined by visible absorption using the extinction coefficients of 2×10⁴ for acrylodan at 391 nm and 2.5×10⁴ for IANBD

B. Construction and Analysis of NM Mutants

To investigate the structural requirements for amyloid fiber assembly, we used yeast Sup35p's NM-region as a model protein. Until recently, fiber assembly kinetics of NM and other amyloid forming proteins have been monitored by binding of dyes such as CongoRed (CR) or ThioflavinT. To gain further insight into NM folding and fiber assembly, a more sensitive method for detecting structural changes, such as that provided by intrinsic fluorescence, was necessary. As NM naturally lacks tryptophan, the only native amino acid with a reasonable environmental-sensitive fluorescence, site-directed mutagenesis could have been employed to artificially introduce tryptophan in NM. However, to improve experimental flexibility we introduced single cysteine substitutions throughout NM. Since NM naturally lacks cysteine, such single point mutations would allow probing of NM folding and assembly in a specific, well defined manner after cross-linking of fluorescent probes to the sulfhydryl-groups of cysteines.

NM mutants with single cysteine replacements at amino acids throughout NM that were predicted to be in structured regions or that were likely involved in the fiber assembly process were constructed. These included the following fifteen mutants: NM^{S2C}, NM^{Y35C}, NM^{Q38C}, NM^{Q40C}, NM^{G43C}, NM^{G68C}, NM^{M124C}, NM^{P138C}, NM^{L144C}, NM^{T158C}, NM^{E167C}, NM^{K184C}, NM^{E203C}, NM^{S234C}, and NM^{L238C}. As indicated in table 1 below, three of the fifteen mutants, NM^{Y35C}, NM^{Q40C}, and NM^{M124C}, were not stably expressed at a sufficiently high protein levels in E. coli. All other mutants were purified to homogeneity under denaturing conditions. To confirm that refolded NM attained a native protein structure, a GST-NM fusion protein was purified with thrombin, and GST was removed by binding to Glutathione-Sepharose. A structural comparison of refolded and native NM using far-UV circular dichroism (CD) showed no apparent differences between the two proteins.

TABLE 1

| | | Secondary | | Fiber |
|---|---|---|---|---|
| NM | Expression | Structure | Fiber assembly | morphology |
| Protein | in E. coli | [0_{222 nm}] | (CR-binding) | (EM) |
| wild- | yes | −2950 | yes | smooth fibers |
| type | | | | up to 35 μm |
| (wt) | | | | long |
| NM | | | | |
| NM^{S2C} | yes | as wt | as wt | as wt |
| NM^{Y35C} | not | — | — | — |
| | detectable | | | |
| NM^{Q38C} | yes | as wt | as wt | as wt |
| NM^{Q40C} | very low, | — | — | — |
| | not stable | | | |
| NM^{G43C} | yes | −6420 | slower assembly | short fibers, |
| | | | rate | only few are |
| | | | | longer than |
| | | | | 1 μm |
| NM^{G68C} | yes | −6250 | slower assembly | short fibers, |
| | | | rate | only few are |
| | | | | longer than |
| | | | | 1 μm |
| NM^{M124C} | very low, | — | — | — |
| | not stable | | | |
| NM^{P138C} | yes | −4570 | as wt | as wt |
| NM^{L144C} | yes | −4198 | as wt | as wt |
| NM^{T158C} | yes | as wt | as wt | as wt |
| NM^{E167C} | yes | as wt | as wt | as wt |
| NM^{K184C} | yes | −4400 | as wt | as wt |
| NM^{E203C} | yes | −4000 | as wt | less smooth, |
| | | | | many short |
| | | | | fibers |
| NM^{S234C} | yes | −6410 | slower assembly | many short |
| | | | rate | fibers |
| NM^{L238C} | yes | −3730 | no | no detectable |
| | | | | fibers |

To determine the direct influence of individual cysteine replacements on the folding and assembly of NM in vitro, the secondary structure of each NM^{cys} was compared to wild-type NM structure by far-UV CD after refolding. The results are summarized in table 1. Structurally, only NM^{S2C}, NM^{Q38C}, NM^{T158C}, and NM^{E167C} were identical to wild-type NM. All other mutants contained a higher content of secondary structure as indicated by an increased mean residue ellipiticity at [θ]₂₂₂ₙₘ. NM and all ^{Nmcys}, with the exception of NM^{L238C}, had identical mean residue ellipiticities at [θ]^{208nm} of −9000 degree cm² dmol⁻¹. In contrast, NM^{L238C} had a decreased mean residue ellipiticity at [θ]^{208nm} indicating that this mutant had an aberrant structure in comparison to wild-type NM than the other NM^{cys}.

Next, fiber assembly of each mutant was performed on a roller drum and compared to wild-type NM assembly kinetics by binding of CongoRed (CR), which shows a spectral shift after interacting with amyloid fibers. Results form these experiments are summarized in table 1. Only NM^{L238C} did not bind CR under all conditions tested. NM^{G43C}, NM^{G68C}, and NM^{S234C} showed slightly altered CR-binding kinetics suggesting slower fiber assembly rates in comparison to wild-type NM.

Electron microscopy (EM) was used to confirm that NM^{cys} fibers were morphologically identical to wild-type fibers. As indicated in table 1, the electron micrographs showed no apparent differences in fiber density, fiber diameter, or other morphological features in comparison to wild-type NM for NM^{S2C}, NM^{Q38C}, NM^{0138C}, NM^{L144C}, NM^{T58C}, NM^{E67C}, and NM^{K184C}, NM^{L238C} fibers were not detectable by EM, suggesting that the apparent lack of CR-binding of NM^{L238C} was not due to structural differences in fibers that affected CR-binding. Results from CD (secondary structure), CR-binding (fiber assembly kinetics), and EM (fiber morphology) indicate that the NM^{S2C}, NM^{Q38C}, NM^{T158C}, and NM^{E167C} mutants display no apparent differences to wild-type NM with respect to these parameters. To further confirm that the chosen cysteine mutants were not influencing the principal properties of NM, genomic wild-type NM could be replaced by Nm^{cys}.

C. Covalent Binding of Fluorescent Labels to NM^{cys}

Environmentally sensitive fluorescent probes, such as naphthalene derivatives or benzofurazans, are commonly used to detect conformational changes and assembly processes of proteins. Here, we made use of 6-acryloyl-2-dimethylaminonaphathlene (acrylodan) and N,N′-dimethyl-N-(iodoacetyl)-N′-(7-nitrobenz-2-oxa-1,3-diazol-4-yl)ethylene diamine (IANBD amide) both of which react specifically with free thiol-groups on proteins. Whereas acrylodan is very sensitive to its structural environment, IANBD amide exhibits appreciable fluorescence when linked to buried or unsolvated thiols. Therefore, the latter fluorescence is highly sensitive to changes in the solvation level of the fluorophore as seen in folding events, whereas acrylodan is more powerful for investigating conformational changes of a protein. The specific labeling efficiencies of soluble NM^{cys} were in the range of 0.40 to 0.78 (mol label/mol protein) with unspecific binding below 0.05 mol/mol for both fluorescent probes.

After covalent binding to NM^{cys}, the influence of the fluorescent labels on fiber assembly was investigated. No differences were found in fiber assembly for 7 mutants (see table 1) in the presence of fluorescent labels in comparison to non-labeled protein as detected by CR-binding. No gross structural changes in assembled fibers were visible by EM for NM^{Q38C}, NM^{P138C}, NM^{L144C}, NM^{T158C}, NM^{E167C}, and NM^{K184C}. In contrast, NM^{S2C} fibers labeled with both acrylodan and IANBD amide appeared rougher with an overall shorter length, although these changes were subtle.

To determine the incorporation of labeled NM^{cys} into fibers, equal amounts of labeled and non-labeled protein were mixed. The amount of label in the soluble protein fraction was detected over the course of fiber assembly. During the experiment, the label to protein ratio was constant indicating an equal incorporation of labeled and non-labeled protein into fibers. The resulting fibers were monitored for fluorescent emission of the respective label. Both measurements showed that fluorescent-labeled protein was sufficiently incorporated into amyloid fibers without influencing the assembly kinetics or the assembled state for NM^{Q38C}, NM^{P138C}, NM^{L144C}, NM^{T158C}, NM^{E167C}, and NM^{K184C}.

The foregoing experiments examined the folding process of NM using NM^{cys} mutants that exhibited folding processes and structural characteristics similar to wild-type NM. These results provide a better understanding of the process of NM folding.

### EXAMPLE 10

### Kinetic Analysis of Fiber Elongation

The following experiments were performed to characterize how nuclei mediate the conversion of soluble NM to the amyloid form in the elongation phase of fiber formation.

Effect of Fluoresecent Labeling

To determine if fluorescent labels themselves affected fiber assembly, mixed assembly reactions were performed with equal quantities of labeled and unlabeled protein of each mutant. The ratio of labeled protein to unlabeled protein that remained in the soluble phase was constant throughout the assembly time course, and the final level of assembly was the same. The fibers formed with each of the labeled NM^{cys} mutants were indistinguishable from unlabeled NM^{cys} fibers in terms of their diameter (11.5±1.5 nm) and concentration. Thus, covalent attachment of acrylodan/IANBD amide to cysteines did not influence the assembly of these mutants.

Fluorescence Assay for Conformational Conversion

Next, it was investigated which residues f the NM residues are located in positions that would provide a change in fluorescent signal (upon fiber assembly) in conformational conversion reactions (during seeded fiber elongation). For NM^{S2C}, NM^{Q38C}, NM^{T158C}, and NM^{E167C}, cysteine-linked acrylodan showed a blue shift in fluorescence emission maximum (λₘₐₓ), indicating that the environment of each cysteine substitution changed. To determine if these changes were based on the conformnational transitions that are associated with the transition from soluble protein into fibers, fluorescent changes were analyzed for 12 hours in undisturbed, non-seeded reactions. Such reactions depend upon spontaneous nucleation and no NM fibers are detected in this time frame. This experiment revealed that acrylodan fluorescence emission showed a gradual change of λₘₐₓ during the pre-assembly stage for NM^{S2C} and NM^{Q38C}.

By many criteria, the N-region of NM has been established as the region responsible for nucleation. Thus, these changes most likely reflect early conformational transitions involved in the first stage of nucleated conformational conversion (NCC). Acrylodan fluorescence emission of NM^{T158}C and NM^{E167C} revealed no significant change after 12 hours in non-seeded samples (Both of these residues are located in the M-region.). However, coincident with seeded fiber assembly, solutions of NM^{T158C}- and NM^{E167C}-acrylodan showed increased fluorescence intensities accompanied by a blue shift of λₘₐₓ (NM^{T158C}: 521 nm to 486 nm, FIG. 2A; NM^{E167C}: 528 nm to 502 nm). Thus, acrylodan labels at cysteine 158 and 167 are sensitive to the conformational differences between soluble and fibrous NM.

Seeded Elongation Occurs in Two Steps

Both NM^{T158C}- and NM^{E167C}-acrylodan (2 μM each) showed a rate of fiber assembly of ν^{fluor}=8±0.4×10⁻⁴ μmol s⁻¹ at 25° C. in the presence of seed (4% w/w), at which seed concentration soluble NM is present in excess over the seeding fiber ends by approximately 50,000 fold. This fiber assembly rate was similar to that measured for NM^{wt} by far-UV CD (3×10⁻⁴ umol s⁻¹) and light scattering (5±0.3×10⁻⁴ μmol s⁻¹) at identical experimental conditions. To determine the kinetic parameters of fiber assembly it was essential to ensure that both the substrate and the seed were in excess in the reactions. To do this, fiber assembly rates was determined with constant seed concentrations (4% w/w calculated for a 5 μM protein concentration) and varying soluble protein concentrations. Decreasing the soluble NM concentration 100-fold only decreased fiber assembly rates by a factor of two. Hence, soluble protein is in excess with 4% w/w seed and 5 μM soluble NM.

The kinetics of seeded fiber elongation reproducibly showed a lag-phase of 80±10 s at 25° C., then exhibited linear kinetics. The fact that fiber assembly did not begin immediately suggested that an assembly intermediate is formed. Non-fibrous NM is soluble in SDS while fibrous NM shows SDS-resistance. Based on this fact, an assay was developed to detect intermediate complexes, which identifies soluble NM that is associated with seed but still not converted into the fiber state. Seeds were prepared from NM^{K184C}, a cysteine substitution mutant with surface accessible sulfhydryl groups that allow for labeling after fiber formation and that shows a seeding efficiency indistinguishable to that of NM^{wt}, and these NM^{K184C} seeds were biotinylated. Further, NM^{T158C} was labeled with iodo[1-¹⁴C]acetamide. Reactions were started by addition of biotinylated NM^{K184C} seed (50% (w/w)) to soluble NM^{T158C}-iodo[1-¹⁴C] acetamide and at distinct time points aliquots of the reaction were taken and incubated with Streptavidin-coated Dynabeads. A high ratio of seed to soluble protein was used to ensure that the fiber ends (i.e. the seeds) were saturated with soluble NM, which would therefore allow us the best opportunity of observing short-lived intermediate complexes. The beads were removed at different time points using a magnet and washed with SDS to detect non-converted intermediates. Both the SDS soluble protein and the SDS resistant fiber, which were attached to the beads, were analyzed by scintillation counting. It took 30 seconds to collect the beads. At early time points a substantial fraction (˜50%) of the NM assembled with bead-bound seeds was soluble in SDS, at later time points the fraction of SDS-soluble material diminished. In a control experiment, in which the NM^{K184C} seeds were not biotinylated, no radioactivity could be detected attached to the beads. The ability to capture material bound to the seed that had not completely converted, established the formation of a detergent susceptible complex. However, this method did not have sufficient resolving power to analyze kinetic parameters of the assembly process.

To establish kinetic parameters, it was necessary to precisely discriminate between soluble and seed-bound NM. Therefore a sedimentation assay was developed to detect the disappearance of soluble NM^{T158C}-acrylodan during fiber assembly. The total acrylodan concentration was plotted against the acrylodan concentration in the supernatant, and each measurement was repeated 6 times to estimate the level of variation. In combination with the wavelength shift assay described above, this provided sufficient data to kinetically analyze fiber assembly and develop a model for nucleated fiber elongation. These reactions have several components: two reactants—the seed and the soluble NM, with the soluble NM as the substrate being in excess of the seed, and a catalyst that is not used up as the reaction progresses (the catalyst is the fiber ends, which are bound to by the soluble NM, but the same number of ends are present as the fiber elongates). These components and the fact that these reactions reach steady state kinetics suggest that they can be analyzed with the same mathematical formula that has been used to describe enzyme kinetics—the Michaelis-Menten equation: 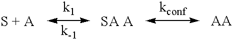
where S is soluble NM, A is assembled protein (seed), SA is bound but not converted intermediate (akin to an enzyme:substrate complex), and AA is converted fiber, which again can act as seed. Importantly, we were unable to discriminate whether seed associates with monomers or oligomers or both. The observed rate of conformational conversion is determined experimentally by k₁, k₋₁, k_{conf}. k₁ and k₋₁ represent the rate constants for binding and dissociation, and k_{conf} is the first-order conformational conversion rate. Since the dissociation rate of converted protein from the amyloid fibers is too slow to be detected in our experimental set-up, the back reaction
   AA→SA
is quasi-irreversible and ignored in our model.

Next, we analyzed our experimental data using a Lineweaver-Burk plot in order to gain more information on the kinetic parameters of fiber assembly. In these experimental conditions, the Lineweaver-Burk plot yielded a straight line and a protein concentration of Kₘ=0.12±0.01 μM, at which the rate of reaction is equal to one half of the limiting rate (maximum rate). We also calculated a maximal rate of conformational conversion Vₘₐₓ=10±0.3×10⁻⁴ μmol s⁻¹, the rate constant of conformational conversion of k_{conf}=5±0.1×10⁻³ s^{-1,} and a conformational conversion efficiency of k_{conf}/Kₘ=42000 M⁻¹ s⁻¹, which is equivalent to an enzyme's specificity constant.

Influences of Temperature on Seeded Fiber Elongation

The effect of increased temperature on seeded fiber elongation was investigated with NM^{T158C}-acrylodan in the presence of 4% w/w seed. A low temperature optimum of the rate of fiber assembly as seen in the logarithm of NCC velocities plotted against the reciprocal temperature (Arrhenius plot) was found. The sticking probability of soluble protein, which is reflected by k_{conf}/k₋₁, characterizes the rate at which soluble NM (S) associates with seed (SA) relative to dissociation, i.e., the sticking probability is high if k₋₁<k_{conf}. In these experiments the abnormal temperature dependence with decreasing ratios of k_{conf}/k₋₁ at elevated temperature indicates a significant rate enhancement for the dissociation of the seed-NM (SA) complex in comparison to its conversion into an assembled fiber (AA). At low temperature k₋₁<<k_{conf} and k_{conf}/Kₘ becomes equal to k₁. Because the dissociation of non-converted, but seed-bound NM, has a high activation energy, k₋₁ becomes predominant at high temperature.

In order to test this experimentally, the velocities of fiber elongation at 25° C. and 40° C. were measured with a constant soluble NM^{T158C}-acrylodan concentration (2 μM) and increasing seed concentrations. It was confirmed that increasing seed concentrations led to increasing fiber elongation velocities at both temperatures yielding maximal elongation rates above 10% w/w of seed. Therefore, fiber elongation velocities at 12% w/w seed, which should be not-rate limiting seed concentrations for fiber elongation, were plotted against the reciprocal temperature. The plot revealed a temperature dependence of fiber elongation that is consistent with the collision theory of Arrhenius. The Arrhenius plot gives a straight line and its slope is equivalent to the activation energy E_{α} divided by the gas constant R=8.3145 J K⁻¹ mol⁻¹. Using this equation, the activation energy for fiber elongation was calculated to be E_{α}=11.7 0.2 kJ mol⁻¹.

Acquisition of Secondary and Tertiary Structure of Soluble NM

In order to elucidate the influence of the conformation of soluble NM on the association with seed, we investigated the rate at which secondary, tertiary, and quaternary structures were acquired in soluble material. When NM is first diluted out of denaturants such as urea or guanidinium chloride (GdmCl), it adopts the characteristics of a molecule that is rich in random coil but partially structured (typical for intrinsically unstructured proteins) indistinguishable from that of NM purified under non-denaturing conditions. To analyze whether the rate of this process influences seeded fiber assembly, 6M GdmCl was used to form a homogenous and monomeric population of denatured NM. After dilution into 5 mM sodium phosphate, pH 7.4, 150 mM NaCl, the time course of far-UV Circular Dichroism (CD) changes at 222 nm was monitored. The acquisition of secondary structure reached half maximal amplitude after 24±2 s with a rate-constant of k_{gain}^{farUV}=2.1±0.2×10⁻² s⁻¹. Thus the formation of secondary structure is not rate determining for seeded fiber elongation.

The kinetics of acquisition of NM tertiary structure was investigated by the four fluorescently-labeled NM^{cys} mutants. Changes in tertiary structure of NM upon dilution into buffer from 6M GdmCl were investigated with two different techniques: IANDB-amide labeled protein was investigated by fluorescence emission and acrylodan labeled protein with near-UV CD. The fluorescence emission of IANBD-amide revealed solvent exposure in all four mutants in 6M GdmCl, as expected. A stable IANDB-amide emission signal was reached after dilution into buffer indicative of a higher ordered environment. The time course had a half maximal amplitude at 31 4 s and a rate constant of k_{gain}^{fluor}=1.6±0.2×10⁻² s⁻¹. Similarly, near UV-CD time courses with acrylodan-labeled NM (all four mutants led to the same results) showed a half maximal amplitude after 33±2 s and a rate constant of k_{gain}^{nearUV}=1.5±0.1×10⁻² s⁻¹. Both independent measurements revealed that formation of some tertiary structure is also not rate limiting for seeded fiber assembly under the experimental conditions chosen.

Quaternary Structure Analysis

Dilution of NM^{wt} out of denaturant led to the formation of a mixed population of monomers and oligomers. 87±5% of NM was monomeric and the remaining fraction heterogeneously oligomeric with varying molecular masses from tetramers to 30mers. Oligomerization was preceded by a lag phase of approximately 60 seconds after dilution out of denaturant, which may suggest that some acquisition of secondary and tertiary structure is required prior to oligomerisation. Populations of monomers and oligomers were established after of a half time of 75±5 seconds and remained constant for 3 hours. Since this steady state was achieved far before spontaneous nucleation (and well before seed was added), NM oligomerisation is not likely to be rate-determining for seeded fiber assembly in our experiments.

The data suggested the following mechanism for initial structural changes of soluble NM, starting from the denatured state: 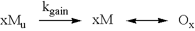
where Mᵤ is the unfolded monomer, M is the random-coil monomer with some structure, and Oₓ are the oligomers. The rate constant for structural gain of monomeric NM from the denatured state was k_{gain}=1.5±0.2×10⁻² s⁻¹. Remarkably, the rate of oligomerisation and establishment of a steady state distribution of monomers and oligomers showed little dependence on the concentration of NM between 0.7 μM and 46 μM NM. This observation agrees with that of a previous study that NM fiber assembly proceeds via the conversion of oligomers to nuclei with little concentration dependence. Nuclei form by conformational rearrangements of NM within the context of oligomeric intermediates and not by assembly of structurally converted monomers.

### EXAMPLE 11

### Bi-directional Formation of Fibers Composed of the Prion-determining Region (NM) of Yeast Sup35p

The following experiments were performed to demonstrate that fibers composed of the NM region of Sup35p are capable of adding NM protein at both ends of the fiber. This was investigated using a mutant NM protein, in which the lysine residue at position 184 was substituted by cysteine, that was capable of forming fibers labeled with specifically modified gold colloids. Visualization of the gold-labeled fibers allowed determination of the directionality of fiber growth.

A. Determining the Accessibility of Cysteine Residues in Assembled Fibers

First, the accessibility of cysteine residues was assayed in fibers composed of cysteine-substituted mutant NM (NM^{cys})proteins, each of which carried different single cysteine replacements at amino acid residues throughout the NM protein. All Nm^{cys}, described in Example 9 above, that formed fibers were examined. For fiber assembly, NM^{cys} protein was diluted out of 4M Gdm*Cl 80-fold into 5 mM potassium phosphate (pH 7.4), 150 mM NaCl to yield a final NM^{cys} protein concentration of 10 μM. To accelerate the rate of fiber assembly, all NM^{cys} proteins were incubated on a roller drum (9 rpm) for 12 hours. The resulting fibers were sonicated with a Sonic Dismembrator Model 302 (Artek) using an intermediate tip for 15 seconds. Sonication resulted in small sized fibers that did not reassemble to larger fibers as determined by electron microscopy (EM). Seeding of fiber assembly was performed by addition of 1% (v/v) of the sonicated fibers to soluble NM^{cys} protein.

To test the accessibility of cysteines in assembled fibers composed of NM^{cys} proteins, EZ-link PEO-maleimide-conjugated biotin (Pierce, product number 21901) was added to the assembled fibers and the labeling efficiency of the biotin was assayed. EZ-liink PEO-maleimide-conjugated biotin was covalently linked to assembled NM^{cys} fibers for 2 hours at 25° C. according to the manufacturer's protocol (protocol number 0748). Remaining free biotin was removed by size exclusion chromatography using D-Salt Excellulose desalting columns (Pierce, product number 20450). Labeling efficiency was determined by competing for avidin binding between biotin and [2-(4′-hydroxybenzene)] benzoic acid (HABA). The binding of HABA to avidin results in a specific absorption band at 500 nm. Since biotin displaces the HABA dye due to higher affinity of biotin for avidin, as compared to that of HABA dye for avidin, the binding of HABA to avidin and thus the specific absorption at 500 nm decreases proportionately when biotin is added to the reaction. Results from this assay indicated that fibers composed of either NM^{cys} proteins in which the lysine residue at position 184 was substituted by a cysteine residue (K184C) or NM^{cys} proteins in which the serine residue at position 2 was substituted by a cysteine residue (S2C), bound a detectable amount of biotin. S2C fibers had a labeling efficiency of 0.16 mol biotin/mol protein, and K184C fibers exhibited a labeling efficiency of 0.56 mol biotin/mol protein. Thus, the cysteine residue at position 184 is highly accessible and the cysteine residue at position 2 is partially accessible on the surface of assembled fibers.

B. Analysis of Fiber Growth Using EM

K184C sonicated fibers were tested for their ability to seed fiber assembly of soluble wild-type NM protein. Fiber assembly was performed as described above using sonicated K184C fibers as seeds to assemble soluble wild-type NM protein. The rate of fiber assembly was assayed by CongoRed binding (CR-binding) and fiber morphology was examined by EM. For EM studies, protein solutions were negatively stained as previously described in Spiess et al., 1987, Electron Microscopy and Molecular Biology: A Practical Approach, Oxford Press, p.147-166. Images were obtained with a CM120 Transmission Electron Microscope (Phillips) with an LaB6 filament, operating at 120 V in low dose mode at a magnification of 4500× and recorded on Kodak SO163 film. Results from CR-binding and EM experiments show that K184C fibers are able to seed wild-type NM fiber assembly. The resulting mixed K184C/NM fibers showed no apparent differences in assembly rate or morphology to fibers seeded with sonicated wild-type NM fibers. Similar results were obtained when biotinylated K184C seeds were used fro fiber assembly.

The surface exposure of the cysteine at position 184 in assembled fibers composed of the K184C mutant protein allowed sufficient labeling of fibers with specifically modified gold colloids. Monomaleimido Nanogold™ (Nanoprobes, product number 2020A) with a particle diameter of 1.4 nm was covalently cross-linked to the sulfhydryl group of accessible cysteine residues in sonicated K184C fibers for 18 hours at 4° C. according to the manufacturer's protocol. Remaining free Nanogold™ was removed by a repeated size exclusion chromatography using D-Salt Excellulose desalting columns (Pierce, product number 20450). The extent of labeling was determined by UV/visible absorption using extinction coefficients for Nanogold™ of 2.25×10⁵ at 280 nm and 1.12×10⁵ at 420 nm. Ratios of optical densities at 280 nm and 420 nm allowed an approximation of the labeling efficiency. These gold-labeled fibers were employed to seed fiber growth of soluble wild-type NM protein.

To visualize the 104 nm Nanogold™ particles attached to the assembled mixed K184C/NM fibers, we used Goldenhance™ (Nanoprobes) according to the manufacturer's instructions. Briefly, equal volumes of enhancer (Solution A) and activator (Solution B) were combined and incubated for 15 min at room temperature. Initiator (Solution C) was then added at a volume equal to that of enhancer or activator, and the resulting mixture was diluted (1:2) with phosphate buffer (Solution D). The final solution acts as an enhancing reagent by selectively depositing gold onto Nanogold™ particles, thereby providing enlargement of Nanogold™ to give electron-dense enlarged Nanogold™ particles in the electron microscope. For negative staining of gold-labeled fibers, 6 μl of protein (8 μM, 1% (w/w) gold labeled seed) were applied to a 400 mesh carbon-coated copper grid (Ted Pella) for 45 seconds. After washing with 100 μl phosphate buffer, grids were incubated with the final Goldenhance™ enhancing reagent, prepared as described above, for 5 min. After washing with 200 μl glass-distilled water, negative staining was employed as in Spiess et al., 1987 Electron Microscopy and Molecular Biology: A Practical Approach, Oxford Press, p.147-166. EM results revealed that the gold-labeled K184C regions are located in the middle of the assembled K184C/NM fibers indicating bi-directional fiber assembly with no apparent polarity in the seeds used.

The foregoing experiments show that fiber assembly of NM proteins occurs at both ends of the fibers. These analyses were performed using K184C, a NM^{cys} mutant wherein the lysine residue at position 184 has been substituted with a cysteine residue. Experiments by biotin-labeling of the cysteine residues on assembled K184C fibers were carried out to determine accessibility of the cysteines. Since wild-type NM protein does not contain any cysteine residues, labeling can only occur at position 184. Results show that position 184 is highly accessible in assembled K184C fibers. The ability of specifically modified gold colloids to covalently cross-link the sulfhydryl group of cysteines enabled generation of gold-labeled fibers that can be visualized by EM. Examination of fiber assembly, by taking advantage of the ability of K184C to produce gold-labeled fibers, indicates that fiber growth occurs bi-directionally. It further indicates that fibers with specific modifications and attachments, a single fiber containing modified and unmodified regions, and mixtures of modified and unmodified fibers can be produced.

### EXAMPLE 12

### Conducting Nanowires Built by Controlled Self-assembly of Amyloid Fibers and Selective Metal Deposition

The following experiments were performed to demonstrate that fibers composed of the NM region of Sup35p can be modified to conduct electricity. This was investigated using a mutant NM protein, in which the lysine residue at position 184 was substituted by cysteine, that was capable of forming fibers labeled with specifically modified gold colloids. These fibers were placed across gold electrodes, and additional metal was deposited by highly specific chemical enhancement of the colloidal gold by reductive deposition of metallic silver and gold from salts. The resulting silver and gold wires were ≈100 nm wide. These biotemplated metal wires demonstrated the conductive properties of a solid metal wire, such as low resistance and ohmic behavior.

A. Materials and Methods

Protein Expression and Purification.

NM and NM^{K184C} was recombinantly expressed in Escherichia coli BL21 [DE3] as described (Scheibel, T., et al., Curr. Biol. 11: 366-369 (2001)) and purified by chromatography with Q-Sepharose (Amersham Pharmacia), hydroxyapatite (Bio-Rad), and Poros HQ (Roche Molecular Biochemicals) as a final step. All purification steps were performed in the presence of 8 M urea.

Fiber Assembly.

Solutions with protein (NM or NM^{K184}C) concentrations >25 μM were rotated at 60 rpm to increase turbulence and surface area. At this protein concentration, many seeding events initiate simultaneous fiber assembly, which results in many short fibers (average fiber length from 60 to 200 nm). These short fibers were then used to seed further soluble NM. The polymerization of NM is a two-stage process that starts with the formation of a nucleus that contains protein with a different conformation than that of soluble protein. The nucleus promotes the conformational conversion of the remaining soluble protein into amyloid fibers. When denatured NM is initially diluted into physiological buffers it has the features of an intrinsically unstructured (random coil-rich) protein. After a lag phase, nuclei form and initiate the rapid conversion of soluble NM into β-sheet-rich amyloid. This second stage can be imitated by addition of pre-formed fibers (seed) to soluble NM. Fibers of different average length were generated by changing the ratios of seed to soluble NM (keeping the soluble NM concentration constantly at 5 μM).

Analysis of Fiber Structure.

After fiber assembly, three techniques were used to examine the fibrous state of NM: far-UV CD (far-ultra-violet circular dichroism), Congo red (CR) binding, and atomic force microscopy (AFM). CD spectra were obtained by using a Jasco (Easton, Md.) 715 spectropolarimeter equipped with a temperature control unit. All spectra were taken with a 0.1-cm pathlength quartz cuvette (Hellma, Forest Hills, N.Y.) in 5 mM potassium phosphate (pH 7.4)/150 mM NaCl (standard buffer). The settings for wavelength scans were 5-nm bandwidth; 0.25-sec response time; speed, 20 nm/min; and four accumulations.

CR-binding was carried out as described (Glover, J. R., et al. Cell, 89: 811-819 (1997)). Proteins were diluted to a final concentration of 1 μM into standard buffer plus 10 μM CR and incubated for 1 min at 25° C. before measuring the absorbance at 540 and 477 nm.

Samples for AFM analysis were placed on freshly cleaved mica attached to 15-mm AFM sample disks (Ted Pella, Redding, Calif.). After 3 min of adsorption at 25° C., disks were rinsed once with buffer and twice with Millipore filtered distilled H₂O. The samples were then allowed to air dry. Contact and tapping-mode imaging were performed on a Digital Instruments (Santa Barbara, Calif.) multimode scanning probe microscope (Veeco, Santa Barbara, Calif.) by using long, thin-leg standard silicon nitride (Si₃N₄) probes for contact mode and standard etched silicon probes for tapping mode.

Analysis of Fiber Stability.

To investigate fiber stability at elevated temperatures, NM fibers were incubated in standard buffer for 90 min at 98° C., before assessment by CD, CR binding, and AFM. The stability of the fibers was also tested under other temperatures for varying lengths of time, i.e., several months at 25° C. and after freezing at −20° C. and −80° C. Chemical stability was tested by the addition of high concentrations of salt (2.5 M NaCl) or denaturants [8 M urea or 2 M guanidiniumchloride (Gdm·Cl)] to the standard buffer (5 mM sodium phosphate, pH 6.8) and assessed by CD, CR binding, and AFM. NM fiber stability in strong alkaline or acidic solutions and in organic solvents was tested by immobilizing the fibers on mica, air-drying them, and treating them with NaOH (pH 10), HCl (pH 2), or 100% ethanol for several hours. These conditions were not compatible with CD and CR-binding assessment, therefore only AFM was used.

Gold Toning.

Monomaleimido Nanogold (Nanoprobes, Yaphank, N.Y.) with a particle diameter of 1.4 nm was covalently cross-linked to NM^{K184C} fibers as described in Scheibel, T., et al., Curr. Biol. 11: 366-369 (2001), incorporated by reference. The Nanogold reagent was dissolved in 0.02 ml isopropanol, then diluted to 0.2 ml with deionized water. The activated Nanogold solution was added to the NM^{K184C} fibers and incubated for 2 hours at 25° C. Unbound gold particles were separated from the NM^{K184C} fibers using gel exclusion chromatography. The Nanogold conjugate was effectively isolated using a Pharmacia Superdex 400HR medium (which fractionate a wide range of molecular weights). The 1.4-nm Nanogold particles were then subjected to “gold toning” (i.e., silver enhancement followed by gold enhancement). In this procedure, the Nanogold particles act as promoters for reducing silver ions from a solution. The Nanogold-labeled fibers are subjected to silver enhancement with LI Silver (Nanoprobes) performed according to the manufacturer's protocol: solutions A (enahancer solution) and B (activator solution) were mixed in a 1:1 ratio and incubated with the fibers at 25° C.). The resulting silver-coated fiber-bound Nanogold particles were gold-enhanced with GoldEnhance LM (Nanoprobes). Enhancement was performed according to the manufacturer's protocol: solutions A-D (A: enhancer; B: activator; C: initiator; D: buffer) were mixed in a 1:1:1:1 ratio and incubated with the fibers at 25° C.). Exposure times varied from 3 min of silver enhancement and 3 min of gold enhancement to 25 min of silver enhancement and 25 min of gold enhancement.

Electrode Assembly and Visualization.

Electrodes were prepared on Si₃N₄ membrane substrates as described in Morkved, T. L., et al., Polymer, 39: 3871-3875 (1998), incorporated herein by reference. The electrodes were constructed by spinning polymer resist layers onto Si₃N₄ substrates and exposing them to a scanned electron beam. The electron beam demarcated the electrode sites. The exposed polymer was etched away, and gold vapor was applied to fill the resulting gaps. Finally, the remaining polymer was dissolved away, leaving the gold in the pattern inscribed by the electron beam. Typically, gaps between electrodes were 2-10 μm. Transmission electron microscopy (TEM) images of electrodes in the absence and presence of protein fibers were obtained with a CM120 transmission electron microscope (Phillips, FEI, Hillsboro, Oreg.) with a LaB6 filament, operating at 120 kV in low-dose mode at a magnification of ×45,000, and recorded on Kodak S0163 film. Alternatively, samples were imaged by AFM in contact mode. Conductivity measurements were performed as described (Morkved, T. L., et al., Polymer, 39: 3871-3875 (1998)). Briefly, conductivity measurements were performed by biasing the sample with a constant voltage from a Hewlett Packard function synthesizer and, using Keithley electrometers, measuring current and voltage across the sample over a range of temperatures.

B. NM Fibers Are Highly Stable

To investigate the feasibility of using NM fibers in building nanoscale devices, fiber stability was first evaluated under extreme conditions such as those that might be encountered in industrial manufacturing processes. NM fibers assembled at physiological pH and room temperature were assayed for stability by three techniques that differentiate between NM in its soluble and amyloid state. Far-UV CD distinguishes the β-sheet-rich secondary structure of NM fibers from the random coil-rich structure of soluble NM. CR exhibits a spectral shift when it intercalates into the cross-pleated β-strands of NM fibers, which is not observed with soluble NM. AFM and EM were used to monitor the maintenance of fiber morphology.

NM fibers were incubated in standard buffer (5 mM sodium phosphate, pH 6.8) at high and low temperatures, in the absence or presence of high salt (2.5 M NaCl), and in denaturants (8 M urea or 2 M guanidiniumchloride, Gdm·Cl). By all three techniques, fibers were stable in standard buffer after incubation for 90 min at 98° C., for several months at 25° C., and after freezing at −20° and −80° C. (Some shearing of long fibers occurred with repeated cycles of freeze-thawing.) Fibers were completely stable to prolonged incubation in the absence of salt and at 2.5 M salt. They dissociated in <2 h at concentrations of Gdm·Cl >4 M but remained intact in the presence of 2 M Gdm·Cl and 8 M urea.

To test whether NM fibers can withstand strong alkaline or acidic solutions and incubation in organic solvents, which are incompatible with CD and CR-binding assays, NM fibers were immobilized on mica, imaged by AFM, incubated with test solutions [NaOH (pH 10), HCl (pH 2), or 100% ethanol], at 25° C. for up to 2 hours and then reimaged. No morphological changes were apparent after any of these treatments. Therefore, NM fibers show unusually high chemical and thermal stability for a biological material.

C. Production of NM Fibers of Variable Lengths

Studies of the NM amyloid fibers have provided insights into how fibers assemble and how assembly can be controlled (Glover, J. R., et al. Cell, 89: 811-819 (1997); Serio, T. R., et al.. Science, 289: 1317-1321 (2000); Scheibel, T., et al., Nat. Struct. Biol., 8: 958-962 (2001) all of which are oncorprated by reference). The rate of fiber formation by purified soluble NM is dramatically increased by the addition of preformed NM fibers, which seed assembly from their ends (DePace, A. H., et al., Nat. Struct. Biol., 9: 389-396 (2002); Scheibel, T., et al., Curr. Biol., 11: 366-369 (2001)). Pools of fibers with different average lengths were generated by simple manipulation of the assembly conditions. First, short fibers (60-200 nm) were produced by rotating solutions with high NM protein concentrations (>25 μM) at high speeds (60 rpms) to increase turbulence and surface area. These conditions produced short fibers by greatly increasing the efficiency of seeding (such that it dominates over assembly), rather than by simply shearing fibers after they had assembled. Indeed, when preformed fibers were sheared by the much more physically disruptive force of sonication, the resulting fibers had longer average lengths and a much more heterogeneous distribution. The resulting sonicated fibers showed lengths varying from 100 to 500 nm (Scheibel, T., et al., Curr. Biol., 11: 366-369 (2001)).

The short fibers produced by vigorous rotation of high concentrations of NM were used to seed further soluble NM. By simply changing the ratios of seed to soluble NM and by controlling the assembly temperatures (i.e., for preferred fiber assembly, the temperature was kept constant at 25° C.) fibers of different average length were generated. At seed to soluble NM ratios of 1:1 (wt/wt), fibers showed an average length of 500±100 nm. Increasing the soluble NM concentration increased fiber lengths. At ratios of 1:16 of seed to soluble NM, fibers were ≈5±1 μm long. Ratios of 1:64 led to even longer fibers but these had more variable lengths (10 μm up to several hundred micrometers).

A remarkable phenomenon that was sometimes observed when long fibers were prepared for microscopy was their alignment next to each other without any external manipulation. This alignment varied with the buffers in which fibers were suspended and the manner in which the surfaces were prepared in a fashion that has not been completely deciphered.

D. NM Fibers Are Insulators

To examine the electrical behavior of the protein fibers, Si₃N₄ membrane substrates were grown on a silicon wafer which allowed for in-plane electrode fabrication, low-temperature transport measurements, and direct visualization by TEM (Morkved, T. L., et al., Polymer, 39: 3871-3875 (1998)). The electrodes were constructed by spinning polymer resist layers onto Si₃N₄ substrates and exposing them to a scanned electron beam. The electron beam demarcated the electrode sites. The exposed polymer was etched away, and gold vapor was applied to fill the resulting gaps. Finally, the remaining polymer was dissolved away, leaving the gold in the pattern inscribed by the electron beam. Typically, gaps between electrodes were 2-10 μm. NM fibers with polydispersed lengths (>2 μm) were randomly deposited on the electrodes. Binding of the protein fibers to the electrodes and bridging of the gap between the electrodes were confirmed by AFM. Current (I) and voltage (V) readings were taken as electricity was applied to the electrodes and the I-V curve for bare fibers showed a very high resistance (R>10¹⁴ Ω), with no measurable conductivity. Thus, NM amyloid fibers are by themselves good insulators.

E. NM Fibers Can Be Converted into Conducting Nanowires with Low Ohmic Resistance

NM fibers were converted to conducting nanowires by a multistep process. A derivative of NM was used that was genetically engineered to contain a cysteine residue that remained accessible after fiber formation (See, for example, Examples 9 and 10 above, and (Scheibel, T., et al., Curr. Biol., 11: 366-369 (2001)). This derivative, NM^{K184C}, assembled in vitro with kinetics that were indistinguishable from those of the wild-type protein and led to fibers with the same physical properties. Monomaleimido Nanogold (Nanoprobes), which has the chemical specificity to form covalent links with the sulfhydrl groups of cysteine residues, was covalently cross-linked to NM^{K184C} fibers. The gold particles had a diameter of 1.4 nm and their distribution along the surface of the NM^{K184C} fibers was confirmed by TEM. Importantly, linking Nanogold covalently to NM fibers affected neither fiber stability nor fiber morphology.

As the distance between the NM^{K184C} cysteine residues in a fiber is ≈3-5 nm and the Nanogold particles have a diameter of only 1.4 nm, it was necessary to bridge the particles with metal to gain conductivity. GoldEnhance LM (Nanoprobes) was first used, by which gold ions are deposited from solution onto the preexisting particles of Nanogold, followed by chemical reduction of the gold ions to form metallic gold. This process itself was inefficient in gaining conductivity, because binding and reducing the soluble gold ions did not fill all of the gaps between the covalently linked Nanogold particles as determined by TEM and AFM.

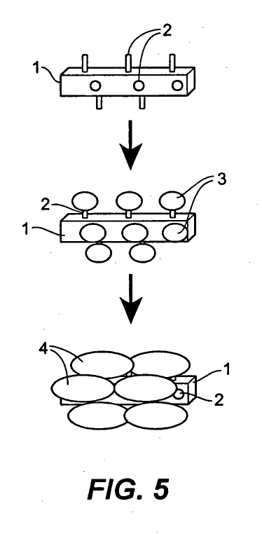

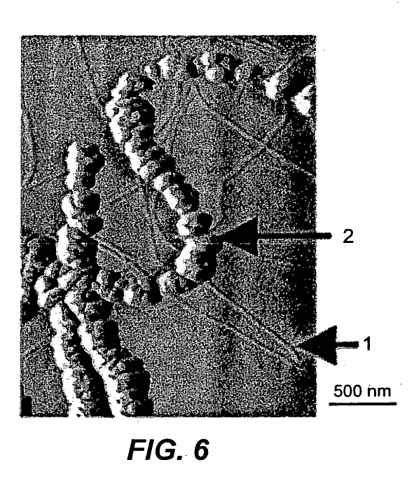

A different enhancement protocol (gold toning, FIG. 5) proved much more efficient. The Nanogold particles (FIG. 5, number 2) on the labeled fibers (FIG. 5, number 1) acted as promoters for reducing silver ions (FIG. 5, number 3) (LI Silver, Nanoprobes) from a solution. The resulting silver-coated fiber-bound Nanogold particles were then gold-enhanced with GoldEnhance LM (FIG. 5, number 4). This gold-toning technique led to fibers with densely packed gold particles. The gold-toned fibers showed a significant increase in diameter from 9-11 nm (bare fibers; FIG. 6, number 1) to 80-200 nm (labeled fibers; FIG. 6, number 2), with the diameter of the resulting fiber strictly depending on the length of exposure time of both the silver and the gold enhancement solution (longer exposure time=thicker fiber). The diameters of the metal wires varied somewhat with different batches of fibers and gold- and silver-toning solutions but were extremely consistent within reactions, i.e., all were within a 10% range. Gold toning was remarkably specific for fibers that had been covalently labeled with Nanogold particles. When NM^{K184C} fibers that were linked to Nanogold were incubated together with a large excess of unlabeled NM^{K184C} fibers, the toning process was restricted to labeled fibers (FIG. 6). Furthermore, the diameters of the wires were consistent within single experiments with fixed exposure times. Therefore, controlling the enhancement exposure time controlled the thickness for the resulting gold wires.

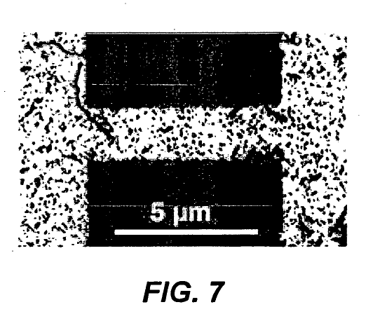

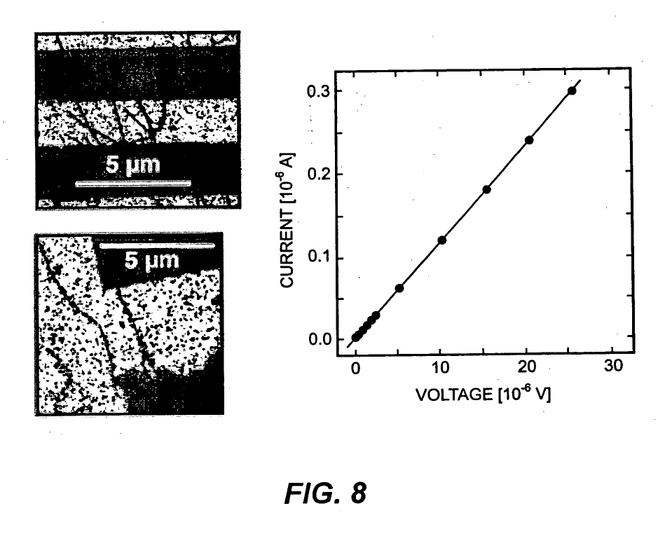

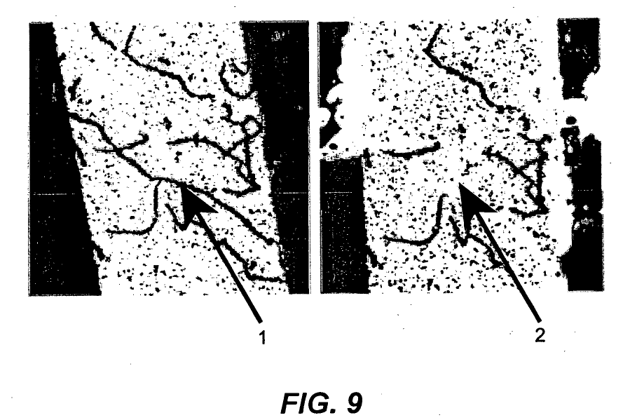

The electrical behavior of NM-templated metallic fibers was assessed by randomly depositing fibers with a length >2 μm and covalently attached Nanogold particles on patterned electrodes, followed by gold toning to form metallically continuous gold nanowires (FIGS. 7-9). Although no background deposition of gold had been detected on unlabeled NM fibers deposited on mica, some gold deposition did occur when enhancement was performed on the Si₃N₄ electrodes. No conductivity was detected in cases where the gold nanowires did not bridge the electrode gap (FIG. 7). In contrast, conductivity was readily detected when single or multiple gold-toned nanowires crossed the gap. I-V curves were linear (FIG. 8), exhibiting ohmic conductivity with low resistance (R=86 Ω for fibers with diameters of 100 nm; this resistance was exhibited in each of six repeated measurements with <1 Ω variation, and with one to four bridging nanowires). The resistance measurements were stable within tenths of ohms within any given fiber (FIG. 8). Such an ohmic response indicates continuous, metallic connections across the sample. The low resistance is that expected for grain-boundary-dominated transport in a polycrystalline metal. In most cases the current was independent of the voltage scan direction and experiments could be repeated several times with the same pair of electrodes and the same nanowire. Notably, in some instances fibers were vaporized (FIG. 9, number 2) from the electrodes when the voltage was increased after the initial conductivity measurements were finished (FIG. 9). This vaporization is a consequence of Joule heating in which the power delivered to the fiber by the current results in a temperature increase sufficient to vaporize the fiber. The Joule heating power depends not only on the applied voltage but also on fiber resistance, which will vary with fiber length and other factors. Bridging fibers (FIG. 9, number 1) were vaporized and did not reassemble, but nonbridging fibers remained. In such cases conductivity was lost on remeasurement. This loss of conductivity confirmed that the bridging fibers were the active nanowires and demonstrated that they can act as fuses at higher voltages and currents.

The foregoing experiments demonstrate that NM protein fibers are excellent candidates for nanocircuit construction. They are exceedingly good insulators without metal coating (R>10¹⁴ Ω) and have very good electrical conductivity with gold and silver coating (R=86 Ω) and linear I-V curves. Previously the least resistance achieved with metallized proteinaceous material was of the order of 200 kΩ, >1,000 times greater than the resistance for metallized NM fibers (Fritzsche, W., et al. Appl. Phys. Lett., 75: 2854-2856 (1999)).

The diameter of the wires produced was 80-200 nm, well below the dimensions accessible by standard electronic manufacturing methods. Having achieved the construction of wires with these dimensions, methods to produce even thinner ones are possible. The thickness of these wires was dictated by the relatively large amounts of silver and gold enhancement that were required to fill the gaps between the Nanogold particles attached to cysteine residues (FIGS. 5 and 6). The sizes of these gaps is reduced by introducing additional cysteines into NM (or using other residues), thus providing more frequent binding sites for the gold particles. Smaller gaps between gold particles will require less enhancement to make contacts continuous, and the resulting wire is thinner. This smaller diameter will allow the manufacture of more intricate circuits and could potentially provide a new model system for quantum confinement and single-electron charging effects when electrons tunnel through restricted pathways (Halperin, W. P., Rev. Mod. Phys., 58: 533-606 (1986); Kastner, M. A., Rev. Mod. Phys., 64: 849-858 (1992); Grabert, H., et al., Single Charge Tunneling (Plenum, New York) (1992); Timp, G. L., ed., Nanotechnology (Springer, New York) (1999)).

### EXAMPLE 13

### Production of Semiconductor Nanowires Built By Controlled Self-assembly of Amyloid Fibers and Selective Seminconducting Material Deposition

The following example describes procedures to produce semiconductor nanowires built by controlled self-assembly of amyloid fibrils and selective seminconducting material deposition.

The Sup35 C terminus (e.g., amino acid 246 to 685) lies externally along the length of Sup35 fibers. Thus by replacing the C terminus with semiconductor binding peptides, and by binding semiconducting materials to those peptides, the fibrils are used to produce continuous self-assembling semiconductor wires.

Peptides with binding sites specific for different semiconductors are isolated using phage-display technology as described by Whaley et al. (Whaley, et al., Nature, 405: 665-668 (2000)) and Mao et al. (Mao et al., Science, 303: 213-217 (2004)), both of which are incorporated herein by reference. Amino acid sequences encoding the peptides identified as having semiconductor binding activity are then attached to the C-terminus of Sup35 NM, as a replacement of substitution for all or part of the wild type Sup35p C-terminus, using recombinant DNA techniques. Alternatively, the peptides identified as having semiconductor binding activity are cross-linked to the native amino acid sequence of the NM region of Sup35p (i.e., the C terminus would not be present).

Subsequently, semiconductor materials such as GaAs, ZnS, CdS, InP and Si are incorporated along the length of NM fibers (using the binding peptides as initial sites of attachment) to produce a continuous semiconductor wire.

While the present invention has been described in terms of specific embodiments, it is understood that variations and modifications will occur to those in the art, all of which are intended as aspects of the present invention. Accordingly, only such limitations as appear in the claims should be placed on the invention.

1. An electrical conductor comprising a fibril having a first location separated from a second location and an electrically conductive material disposed on the fibril between the first location and second location to conduct electricity along the fibril from the first location to the second location.

2. The electrical conductor of claim 1 wherein the fibril comprises polypeptide subunits coalesced into an ordered aggregate.

3. The electrical conductor of claim 2 wherein the fibril is characterized by chemical and thermal stability.

4. The electrical conductor of claim 3 wherein the fibril is stable in 0-8 M urea.

5. The electrical conductor of claim 3 wherein the fibril is stable in 0-2 M guanidinium chloride.

6. The electrical conductor of claim 3 wherein the fibril is stable in 0-2.5 M salt solutions.

7. The electrical conductor of claim 3 wherein the fibril is stable in pH 2-10 solutions.

8. The electrical conductor of claim 3 wherein the fibril is stable in 100% ethanol.

9. The electrical conductor of claim 3 wherein the fibril is stable at −80° C. to 98° C.

10. The electrical conductor of claim 3 wherein the fibril is stable at 0° C. to 98° C.

11. The electrical conductor of claim 3 wherein the fibril is stable at −80° C. to 0° C.

12. The electrical conductor of claim 2 wherein the electrical conductor is characterized by a length of 60 nm to 300 μm, and a diameter of 9 nm to 200 nm.

13. The electrical conductor of claim 2 wherein at least one of the polypeptide subunits comprises a SCHAG amino acid sequence.

14. The electrical conductor of claim 13 wherein 90-100% of the polypeptide subunits comprise a SCHAG amino acid sequence.

15. The electrical conductor of claim 13 wherein the SCHAG amino acid sequence includes at least one amino acid residue having a reactive amino acid side chain.

16. The electrical conductor of claim 13 wherein the SCHAG amino acid sequence includes at least one substitution of an amino acid residue having a reactive amino acid side chain.

17. The electrical conductor of claim 15 wherein the reactive amino acid side chain is exposed to the environment of the fibril to permit attachment of electrically conductive material thereto, and
wherein the electrically conductive material is attached to the fibril at the reactive amino acid side chain.

18. The electrical conductor of claim 16 wherein the reactive amino acid side chain of the substituted amino acid is exposed to the environment of the fibril to permit attachment of electrically conductive material thereto, and
wherein the electrically conductive material is attached to the fibril at the reactive amino acid side chain.

19. The electrical conductor of claim 13 wherein at least 30% of the SCHAG amino acid sequence comprises asparagine or glutamine residues.

20-25. (canceled)

26. The electrical conductor of claim 1 wherein the electrically conductive material comprises a material selected from the group consisting of a metal atom and a semiconductor material.

27. The electrical conductor of claim 26 wherein the a metal atom is selected from the group consisting of gold, silver, nickel, copper, platinum, aluminum, gallium, palladium, iridium, rhodium, tungsten, titanium, zinc, and tin.

28. The electrical conductor of claim 26 wherein the a semiconductor material is selected from the group consisting of GaAs, ZnS, CdS, InP and Si.

29. The electrical conductor of claim 27 wherein the fibril is gold-toned.

30. The electrical conductor of claim 29 wherein the fibril is characterized by a resistance range of 0-100 Ω and linear I-V curves.

31. The electrical conductor of claim 30 wherein the fibril is characterized by a resistance range of 0-100 Ω and linear I-V curves between 0 to 0.3×10⁻⁶ A and between 0-30×10⁻⁶ V.

32. A method of making an electrical conductor comprising the steps of:
(a) making a fibril with first and second separated locations; and
(b) disposing on the fibril an electrically conductive material in an amount effective to conduct electricity along the fibril from the first location to the second location.

33. The method according to claim 32 wherein step (a) comprises providing a solution or suspension of polypeptides that have the ability to coalesce into ordered aggregates, and incubating the solution or suspension under conditions to form fibrils from the polypeptides.

34. The method according to claim 33 comprising rotating the solution or suspension to increase turbulence and surface area, thereby promoting fibril formation.

35. The method according to claim 33 comprising contacting the fibrils with additional soluble or suspended polypeptide under conditions to extend the length of the fibrils.

36. The method according to claim 32 wherein step (b) comprises disposing a substrate on the fibril, and disposing a first electrically conductive material on the substrate.

37. The method according to claim 36 wherein a second electrically conductive material is disposed on the first electrically conductive material.

38-56. (canceled)

57. The method according to claim 32 wherein the fibril comprises polypeptide subunits coalesced into ordered aggregates.

58-70. (canceled)

71. The method according to claim 57 wherein the electrically conductive material comprises a metal atom or a semiconductor material.

72. The method according to claim 71 wherein the electrical conductor material is a metal atom selected from the group consisting of gold, silver, nickel, copper, palladium, iridium, rhodium, tungsten, titanium, zinc, and tin.

73. A fuse comprising an electrical conductor according to claim 1, a first electrode attached to the first position, and a second electrode attached to the second position,
wherein the electrical conductor electrically connects the first electrode to the second electrode.

74. The fuse according to claim 73 wherein the electrical conductor is constructed to fail to conduct electricity when exposed to an electrical current above a first amount.

75. The fuse according to claim 74 wherein the electrical conductor destructs when exposed to an electric current above the first amount.

76. An electrical circuit comprising a source of electricity, one or more circuit elements, and electrical conductors disposed between the source of electricity and the one or more circuit elements, and
wherein at least one of the electrical conductors comprises a fibril and an electrically conductive material disposed on the fibril to conduct electricity along the fibril between the source of electricity and circuit element or between two circuit elements.

77. The electrical circuit of claim 76, wherein one of the one or more circuit elements includes a circuit component selected from the group consisting of a capacitor, an inductor, a resistor, an integrated circuit, an oscillator, a transistor, a diode, a switch, and a fuse.

78. The electrical circuit of claim 76, wherein one of the one or more circuit elements is a passive circuit element.

79. The electrical circuit of claim 76, wherein one of the one or more circuit elements is an active circuit element.


SEQUENCE LISTING (65 sequences)

SEQ ID NO: 1 - DNA, 3321 bp, Saccharomyces cerevisiae
  CDS (739)..(2796)
agaaattaaagctacttacaacaacggtctactacaaattaaggtgcctaaaattgtcaatgacactgaaaagccgaagccaaaaaagaggatcgccattgaggaaatacccgacgaagaattggagtttgaagaaaatcccaaccctacggtagaaaattgaatatcgtatctgtttatacacacatacatacatttatatttataataagcgttaaaatttcggcagaatatctgtcaaccacacaaaaatcatacaacgaatggtatatgcttcatttctttgtttcgcattagctgcgctatttgactcaaattattattttttactaagacgacgcgtcacagtgttcgagtctgtgtcatttcttttgtaattctcttaaaccacttcataaagttgtgaagttcatagcaaaattcttccgcaaaaagatgaatcttagttctcagcccaccaaaagaggtacatgctaagatcatacagaagttattgtcacttcttaccttgctcttaaatgtacattacaaccgggtattatatcttacatcatcgtataatatgatctttctttatggagaaaatttttttttcactcgaccaaagctcccattgcttctgaagagtgtagtgtatattggtacatcttctcttgaaagactccattgtactgtaacaaaaagcggtttcttcatcgacttgctcggaataacatctatatctgcccactagcaacaatgtcggattcaaaccaaggcaacaatcagcaaaactaccagcaatacagccagaacggtaaccaacaacaaggtaacaacagataccaaggttatcaagcttacaatgctcaagcccaacctgcaggtgggtactaccaaaattaccaaggttattctgggtaccaacaaggtggctatcaacagtacaatcccgacgccggttaccagcaacagtataatcctcaaggaggctatcaacagtacaatcctcaaggcggttatcagcagcaattcaatccacaaggtggccgtggaaattacaaaaacttcaactacaataacaatttgcaaggatatcaagctggtttccaaccacagtctcaaggtatgtctttgaacgactttcaaaagcaacaaaagcaggccgctcccaaaccaaagaagactttgaagcttgtctccagttccggtatcaagttggccaatgctaccaagaaggttggcacaaaacctgccgaatctgataagaaagaggaagagaagtctgctgaaaccaaagaaccaactaaagagccaacaaaggtcgaagaaccagttaaaaaggaggagaaaccagtccagactgaagaaaagacggaggaaaaatcggaacttccaaaggtagaagaccttaaaatctctgaatcaacacataataccaacaatgccaatgttaccagtgctgatgccttgatcaaggaacaggaagaagaagtggatgacgaagttgttaacgatatgtttggtggtaaagatcacgtttctttaattttcatgggtcatgttgatgccggtaaatctactatgggtggtaatctactatacttgactggctctgtggataagagaactattgagaaatatgaaagagaagccaaggatgcaggcagacaaggttggtacttgtcatgggtcatggataccaacaaagaagaaagaaatgatggtaagactatcgaagttggtaaggcctactttgaaactgaaaaaaggcgttataccatattggatgctcctggtcataaaatgtacgtttccgagatgatcggtggtgcttctcaagctgatgttggtgttttggtcatttccgccagaaagggtgagtacgaaaccggttttgagagaggtggtcaaactcgtgaacacgccctattggccaagacccaaggtgttaataagatggttgtcgtcgtaaataagatggatgacccaaccgttaactggtctaaggaacgttacgaccaatgtgtgagtaatgtcagcaatttcttgagagcaattggttacaacattaagacagacgttgtatttatgccagtatccggctacagtggtgcaaatttgaaagatcacgtagatccaaaagaatgcccatggtacaccggcccaactctgttagaatatctggatacaatgaaccacgtcgaccgtcacatcaatgctccattcatgttgcctattgccgctaagatgaaggatctaggtaccatcgttgaaggtaaaattgaatccggtcatatcaaaaagggtcaatccaccctactgatgcctaacaaaaccgctgtggaaattcaaaatatttacaacgaaactgaaaatgaagttgatatggctatgtgtggtgagcaagttaaactaagaatcaaaggtgttgaagaagaagacatttcaccaggttttgtactaacatcgccaaagaaccctatcaagagtgttaccaagtttgtagctcaaattgctattgtagaattaaaatctatcatagcagccggtttttcatgtgttatgcatgttcatacagcaattgaagaggtacatattgttaagttattgcacaaattagaaaagggtaccaaccgtaagtcaaagaaaccacctgcttttgctaagaagggtatgaaggtcatcgctgttttagaaactgaagctccagtttgtgtggaaacttaccaagattaccctcaattaggtagattcactttgagagatcaaggtaccacaatagcaattggtaaaattgttaaaattgccgagtaaatttcttgcaaacataagtaaatgcaaacacaataataccgatcataaagcattttcttctatattaaaaaacaaggtttaataaagctgttatatatatatatatatatatagacgtataattagtttagttctttttgtaccatataccataaacaaggtaaacttcacctctcaatatatctagaatttcataaaaatatctagcaaggtttcaactccttcaatcacgttttcatcataacccttccccggcgttatttcagaatgtgcaaaatctattagtgacatggaactcaaagaaccagttgtttttttgtcctttggtccttcgctgcttccctcggcatcatcatcatcatcatcatcattatcatcatcgtcgtcatcatcgtctataaaatcatctcgcataagtttgtcaacatcatttagtaattcccatcgctccgggtctccttcgtaaataaacaaaagactacttgatatcattctaacttcttcttctagcatagtattataaaa

SEQ ID NO: 2 - protein, 685 aa, Saccharomyces cerevisiae
MSDSNQGNNQQNYQQYSQNGNQQQGNNRYQGYQAYNAQAQPAGGYYQNYQGYSGYQQGGYQQYNPDAGYQQQYNPQGGYQQYNPQGGYQQQFNPQGGRGNYKNFNYNNNLQGYQAGFQPQSQGMSLNDFQKQQKQAAPKPKKTLKLVSSSGIKLANATKKVGTKPAESDKKEEEKSAETKEPTKEPTKVEEPVKKEEKPVQTEEKTEEKSELPKVEDLKISESTHNTNNANVTSADALIKEQEEEVDDEVVNDMFGGKDHVSLIFMGHVDAGKSTMGGNLLYLTGSVDKRTIEKYEREAKDAGRQGWYLSWVMDTNKEERNDGKTIEVGKAYFETEKRRYTILDAPGHKMYVSEMIGGASQADVGVLVISARKGEYETGFERGGQTREHALLAKTQGVNKMVVVVNKMDDPTVNWSKERYDQCVSNVSNFLRAIGYNIKTDVVFMPVSGYSGANLKDHVDPKECPWYTGPTLLEYLDTMNHVDRHINAPFMLPIAAKMKDLGTIVEGKIESGHIKKGQSTLLMPNKTAVEIQNIYNETENEVDMAMCGEQVKLRIKGVEEEDISPGFVLTSPKNPIKSVTKFVAQIAIVELKSIIAAGFSCVMHVHTAIEEVHIVKLLHKLEKGTNRKSKKPPAFAKKGMKVIAVLETEAPVCVETYQDYPQLGRFTLRDQGTTIAIGKIVKIAE

SEQ ID NO: 3 - DNA, 1427 bp, Saccharomyces cerevisiae
  CDS (182)..(1246)
ctcgaggttgaaaagaatagcaaaaatctttccttttcaaacagctcatttggaattgtttatagcactgaattgaatcgaagaggaataaagatcccccgtacgaacttctttatttttagtttttcattttttgttattagtcatattgttttaagctgcaaattaagttgtacaccaaatgatgaataacaacggcaaccaagtgtcgaatctctccaatgcgctccgtcaagtaaacataggaaacaggaacagtaatacaaccaccgatcaaagtaatataaattttgaattttcaacaggtgtaaataataataataataacaatagcagtagtaataacaataatgttcaaaacaataacagcggccgcaatggtagccaaaataatgataacgagaataatatcaagaataccttagaacaacatcgacaacaacaacaggcattttcggatatgagtcacgtggagtattccagaattacaaaattttttcaagaacaaccactggagggatatacccttttctctcacaggtctgcgcctaatggattcaaagttgctatagtactaagtgaacttggatttcattataacacaatcttcctagatttcaatcttggcgaacatagggcccccgaatttgtgtctgtgaaccctaatgcaagagttccagctttaatcgatcatggtatggacaacttgtctatttgggaatcaggggcgattttattacatttggtaaataaatattacaaagagactggtaatccattactctggtccgatgatttagctgaccaatcacaaatcaacgcatggttgttcttccaaacgtcagggcatgcgccaatgattggacaagctttacatttcagatacttccattcacaaaagatagcaagtgctgtagaaagatatacggatgaggttagaagagtttacggtgtagtggagatggccttggctgaacgtagagaagcgctggtgatggaattagacacggaaaatgcggctgcatactcagctggtacaacaccaatgtcacaaagtcgtttctttgattatcccgtatggcttgtaggagataaattaactatagcagatttggcctttgtcccatggaataatgtcgtggatagaattggcattaatatcaaaattgaatttccagaagtttacaaatggacgaagcatatgatgagaagacccgcggtcatcaaggcattgcgtggtgaatgaaggctgctttaaaaacaagaaagaaagaagaaggaggaaaagaaggttataagggtatgtatataggcagacaaaaaggaaaattaagtgcaaatataaacaaaaatgtcatagaagtatataatagttttgaaatttctgttgcttctatttattctttgttaccccaaccacagaattc

SEQ ID NO: 4 - protein, 354 aa, Saccharomyces cerevisiae
MMNNNGNQVSNLSNALRQVNIGNRNSNTTTDQSNINFEFSTGVNNNNNNNSSSNNNNVQNNNSGRNGSQNNDNENNIKNTLEQHRQQQQAFSDMSHVEYSRITKFFQEQPLEGYTLFSHRSAPNGFKVAIVLSELGFHYNTIFLDFNLGEHRAPEFVSVNPNARVPALIDHGMDNLSIWESGAILLHLVNKYYKETGNPLLWSDDLADQSQINAWLFFQTSGHAPMIGQALHFRYFHSQKIASAVERYTDEVRRVYGVVEMALAERREALVMELDTENAAAYSAGTTPMSQSRFFDYPVWLVGDKLTIADLAFVPWNNVVDRIGINIKIEFPEVYKWTKHMMRRPAVIKALRGE

SEQ ID NO: 5 - protein, 8 aa, Artificial Sequence
  Description of Artificial Sequence FLAG peptide
DYKDDDDK

SEQ ID NO: 6 - protein, 8 aa, Artificial Sequence
  Description of Artificial Sequence FLAG peptide
DYKDEDDK

SEQ ID NO: 7 - protein, 9 aa, Artificial Sequence
  Description of Artificial Sequence Strep epitope
AWRHPQFGG

SEQ ID NO: 8 - protein, 13 aa, Artificial Sequence
  Description of Artificial Sequence Hemagglutinin epitope
YPYDVPDYAIEGR

SEQ ID NO: 9 - protein, 11 aa, Artificial Sequence
  Description of Artificial Sequence myc epitope
EQKLLSEEDLN

SEQ ID NO: 10 - protein, 9 aa, Saccharomyces cerevisiae
PQGGYQQYN

SEQ ID NO: 11 - DNA, 445 bp, Artificial Sequence
  Description of Artificial Sequence CUP1 promoter
ccattaccgacatttgggcgctatacgtgcatatgttcatgtatgtatctgtatttaaaacacttttgtattatttttcctcatatatgtgtataggtttatacggatgatttaattattacttcaccaccctttatttcaggctgatatcttagccttgttactagttagaaaaagacatttttgctgtcagtcactgtcaagagattcttttgctggcatttcttctagaagcaaaaagagcgatgcgtcttttccgctgaaccgttccagcaaaaaagactaccaacgcaatatggattgtcagaatcatataaaagagaagcaaataactccttgtcttgtatcaattgcattataatatcttcttgttagtgcaatatcatatagaagtcatcgaaatagatattaagaaaaacaaactgtacaatcaatcaatcaatca

SEQ ID NO: 12 - DNA, 717 bp, Aequorea victoria
atgtctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttcggttatggtgttcaatgttttgctagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagagaccacatggtcttgttagaatttgttactgctgctggtattacccatggtatggatgaattgtacaaataa

SEQ ID NO: 13 - DNA, 27 bp, Artificial Sequence
  Description of Artificial Sequence HA tag-encoding sequence
tacccatacgacgtcccagactacgct

SEQ ID NO: 14 - DNA, 645 bp, Artificial Sequence
  Description of Artificial Sequence yeast Sup35Rdelta2-5 encoding sequence
atgtcggattcaaaccaaggcaacaatcagcaaaactaccagcaatacagccagaacggtaaccaacaacaaggtaacaacagataccaaggttatcaagcttacaatgctcaagcccaacctgcaggtgggtactaccaaaattaccaaggttattctgggtacccacaaggtggccgtggaaattacaaaaacttcaactacaataacaatttgcaaggatatcaagctggtttccaaccacagtctcaaggtatgtctttgaacgactttcaaaagcaacaaaagcaggccgctcccaaaccaaagaagactttgaagcttgtctccagttccggtatcaagttggccaatgctaccaagaaggttggcacaaaacctgccgaatctgataagaaagaggaagagaagtctgctgaaaccaaagaaccaactaaagagccaacaaaggtcgaagaaccagttaaaaaggaggagaaaccagtccagactgaagaaaagacggaggaaaaatcggaacttccaaaggtagaagaccttaaaatctctgaatcaacacataataccaacaatgccaatgttaccagtgctgatgccttgatcaaggaacaggaagaagaagtggatgacgaagttgttaacgat

SEQ ID NO: 15 - protein, 215 aa, Artificial Sequence
  Description of Artificial Sequence yeast Sup35Rdelta2-5 encoding sequence
MSDSNQGNNQQNYQQYSQNGNQQQGNNRYQGYQAYNAQAQPAGGYYQNYQGYSGYPQGGRGNYKNFNYNNNLQGYQAGFQPQSQGMSLNDFQKQQKQAAPKPKKTLKLVSSSGIKLANATKKVGTKPAESDKKEEEKSAETKEPTKEPTKVEEPVKKEEKPVQTEEKTEEKSELPKVEDLKISESTHNTNNANVTSADALIKEQEEEVDDEVVND

SEQ ID NO: 16 - DNA, 813 bp, Artificial Sequence
  Description of Artificial Sequence yeast Sup35R2E2 encoding sequence
atgtcggattcaaaccaaggcaacaatcagcaaaactaccagcaatacagccagaacggtaaccaacaacaaggtaacaacagataccaaggttatcaagcttacaatgctcaagcccaacctgcaggtgggtactaccaaaattaccaaggttattctgggtaccaacaaggtggctatcaacagtacaatccccaaggtggctatcaacagtacaatccccaaggtggctatcaacagtacaatcccgacgccggttaccagcaacagtataatcctcaaggaggctatcaacagtacaatcctcaaggcggttatcagcagcaattcaatccacaaggtggccgtggaaattacaaaaacttcaactacaataacaatttgcaaggatatcaagctggtttccaaccacagtctcaaggtatgtctttgaacgactttcaaaagcaacaaaagcaggccgctcccaaaccaaagaagactttgaagcttgtctccagttccggtatcaagttggccaatgctaccaagaaggttggcacaaaacctgccgaatctgataagaaagaggaagagaagtctgctgaaaccaaagaaccaactaaagagccaacaaaggtcgaagaaccagttaaaaaggaggagaaaccagtccagactgaagaaaagacggaggaaaaatcggaacttccaaaggtagaagaccttaaaatctctgaatcaacacataataccaacaatgccaatgttaccagtgctgatgccttgatcaaggaacaggaagaagaagtggatgacgaagttgttaacgat

SEQ ID NO: 17 - protein, 271 aa, Artificial Sequence
  Description of Artificial Sequence yeast Sup35R2E2 encoding sequence
MSDSNQGNNQQNYQQYSQNGNQQQGNNRYQGYQAYNAQAQPAGGYYQNYQGYSGYQQGGYQQYNPQGGYQQYNPQGGYQQYNPDAGYQQQYNPQGGYQQYNPQGGYQQQFNPQGGRGNYKNFNYNNNLQGYQAGFQPQSQGMSLNDFQKQQKQAAPKPKKTLKLVSSSGIKLANATKKVGTKPAESDKKEEEKSAETKEPTKEPTKVEEPVKKEEKPVQTEEKTEEKSELPKVEDLKISESTHNTNNANVTSADALIKEQEEEVDDEVVND

SEQ ID NO: 18 - DNA, 641 bp, MOUSE
  CDS (1)..(633)
atgtctaaaaagcggccaaagcctggagggtggaacaccggtggaagccggtatcccgggcagggaagccctggaggcaaccgttacccacctcagggtggcacctgggggcagccccacggtggtggctggggacaaccccatgggggcagctggggacaacctcatggtggtagttggggtcagccccatggcggtggatggggccaaggagggggtacccataatcagtggaacaagcccagcaaaccaaaaaccaacctcaagcatgtggcaggggctgcggcagctggggcagtagtggggggccttggtggctacatgctggggagcgccgtgagcaggcccatgatccattttggcaacgactgggaggaccgctactaccgtgaaaacatgtaccgctaccctaaccaagtgtactacaggccagtggatcagtacagcaaccagaacaacttcgtgcacgactgcgtcaatatcaccatcaagcagcacacggtcaccaccaccaccaagggggagaacttcaccgagaccgatgtgaagatgatggagcgcgtggtggagcagatgtgcgtcacccagtaccagaaggagtcccaggcctattacgacgggagaagatccagctgataacc

SEQ ID NO: 19 - protein, 211 aa, MOUSE
MSKKRPKPGGWNTGGSRYPGQGSPGGNRYPPQGGTWGQPHGGGWGQPHGGSWGQPHGGSWGQPHGGGWGQGGGTHNQWNKPSKPKTNLKHVAGAAAAGAVVGGLGGYMLGSAVSRPMIHFGNDWEDRYYRENMYRYPNQVYYRPVDQYSNQNNFVHDCVNITIKQHTVTTTTKGENFTETDVKMMERVVEQMCVTQYQKESQAYYDGRRSS

SEQ ID NO: 20 - DNA, 644 bp, Mesocricetus auratus
  CDS (1)..(636)
atgtctaagaagcggccaaagcctggagggtggaacactggcggaagccgataccctgggcagggcagccctggaggcaaccgttacccacctcagggtggcggcacatgggggcaaccccatggtggtggctggggacagccccatggtggtggctggggacagccccatggtggtggctggggtcagccccatggtggtggctggggtcaaggaggtggcacccacaatcagtggaacaagcccagtaagccaaaaaccaacatgaagcacatggccggcgctgctgcggcaggggccgtggtggggggccttggtggctacatgctggggagtgccatgagcaggcccatgatgcattttggcaatgactgggaggaccgctactaccgtgaaaacatgaaccgctaccctaaccaagtgtattaccggccagtggaccagtacaacaaccagaacaactttgtgcacgattgtgtcaacatcaccatcaagcagcacacagtcaccaccaccaccaagggggagaacttcacggagaccgacatcaagataatggagcgcgtggtggagcagatgtgtaccacccagtatcagaaggagtcccaggcctactacgatggaagaaggtccagctgataacc

SEQ ID NO: 21 - protein, 212 aa, Mesocricetus auratus
MSKKRPKPGGWNTGGSRYPGQGSPGGNRYPPQGGGTWGQPHGGGWGQPHGGGWGQPHGGGWGQPHGGGWGQGGGTHNQWNKPSKPKTNMKHMAGAAAAGAVVGGLGGYMLGSAMSRPMMHFGNDWEDRYYRENMNRYPNQVYYRPVDQYNNQNNFVHDCVNITIKQHTVTTTTKGENFTETDIKIMERVVEQMCTTQYQKESQAYYDGRRSS

SEQ ID NO: 22 - protein, 780 aa, Saccharomyces cerevisiae
MKKKDNSDDKDNVASGGYKNAADAGSNNASKKSSYRNWKGGNYGGYSYNSNYNNYNNYNNYNNYNNYNNYNKYNGGYKSTYKSAVTNSGTTSASTTSTSNKSNTSSKCSTDCKNKGKGNSTGKWKVDVSKKKNSVRSAMSNASGKAYNVADCSDKNTVKRAAHADSNCMATCVTDYSSGAKWAKMAASVVDRRDSANDTKDAVVTDVATDKAKGYKTDYVSDNDSRYKVDTDSKVSVKSSSVTVAVTSSVNRSNSSSSRTVVVNTRVNNRNSGKVVDTASVRAKANVKDDADKNKSGRTGRDDHKDKADDSCVKYMNDTVKYMSKTVDSNVNDWKRDTAVGGSDSRVKDHNRAYKRADDGVNTDSAYGSRMNKTNRKGHRYGCGRNGAGKSTMRAANGDGDKDTRTCVHKGGDDVSADSTSRAAASVGDRRATVGSSGGWKMKARAMKADDTNHDVSNVKWYHTDTSVSHDSGDTVCTDHYNKKAYYKGNAAVKAKSYYTTDSNAMRGTGVKSNTRAVAKMTDVTSYGAKSSHVSCSSSSRVACGNGAGKSTKTGVNGKVKHNRGYAHAHVNHKKTANYWRYGDDRVKSRKSDKMMTKDDDGRGKRAAVGRKKKSYVKWKYWKKYNSWVKDVVHGKVKDDHASRGGYRSVTKHDVGDSANHTGSSGGVKVVAGAMWNNHVDTNYDRDSGAAVARDWSGGVVMSHNNVGACWVNGKMVKGSAVDSKDGGNADAVGKASNAKSVDDDDSANKVKRKKRTRNKKARRRRYWSSKGTKVDTDDD

SEQ ID NO: 23 - protein, 1075 aa, Saccharomyces cerevisiae
MDNKRLYNGNLSNIPEVIDPGITIPIYEEDIRNDTRMNTNARSVRVSDKRGRSSSTSPQKIGSYRTRAGRFSDTLTNLLPSISAKLHHSKKSTPVVVVPPTSSTPDSLNSTTYAPRVSSDSFTVATPLSLQSTTTRTRTRNNTVSSQITASSSLTTDVGNATSANIWSANAESNTSSSPLFDYPLATSYFEPLTRFKSTDNYTLPQTAQLNSFLEKNGNPNIWSSAGNSNTDHLNTPIVNRQRSQSQSTTNRVYTDAPYYQQPAQNYQVQVPPRVPKSTSISPVILDDVDPASINWITANQKVPLVNQISALLPTNTISISNVFPLQPTQQHQQNAVNLTSTSLATLCSQYGKVLSARTLRGLNMALVEFSTVESAICALEALQGKELSKVGAPSTVSFARVLPMYEQPLNVNGFNNTPKQPLLQEQLNHGVLNYQLQQSLQQPELQQQPTSFNQPNLTYCNPTQNLSHLQLSSNENEPYPFPLPPPSLSDSKKDILHTISSFKLEYDHLELNHLLQNALKNKGVSDTNYFGPLPEHNSKVPKRKDTFDAPKLRELRKQFDSNSLSTIEMEQLAIVMLDQLPELSSDYLGNTVIQKLFENSSNIIRDIMLRKCNKYLTSMGVHKNGTWVCQKIIKMANTPRQINLVTSGVSDYCTPLFNDQFGNYVIQGILKFGFPWNSFIFESVLSHFWTIVQNRYGSRAVRACLEADSIITQCQLLTITSLIIVLSPYLATDTNGTLLITWLLDTCTLPNKNLILCDKLVNKNLVKLCCHKLGSLTVLKILNLRGGEEEALSKNKIIHAIFDGPISSDSILFQILDEGNYGPTFIYKVLTSRILDNSVRDEAITKIRQLILNSNINLQSRQLLEEVGLSSAGISPKQSSKNHRKQHPQGFHSPGRARGVSVSSVRSSNSRHNSVIQMNNAGPTPALNFNPAPMSEINSYFNNQQVVYSGNQNQNQNGNSNGLDELNSQFDSFRIANGTNLSLPIVNLPNVSNNNNNYNNSGYSSQMNPLSRSVSHNNNNNTNNYNNNDNDNNNNNNNNNNNNNNNNNNNNNSNNSNNNNNNDTSLYRYRSYGY

SEQ ID NO: 24 - protein, 76 aa, Saccharomyces cerevisiae
MSANDYYGGTAGKSYSRSNSSAHNKTRGYYYHGYYNGYNGYNGYNGYNGYNGYNGHVYVRGNGCAACAACCCTMDM

SEQ ID NO: 25 - protein, 380 aa, Saccharomyces cerevisiae
MSSDDNDYGDDKTTTVKKNKAGSGTSDAAASSSNKNNNSNNSSSNNSNDTSSSKDGTANDKGSNDTKNKKSATSANANANASSAGSGWTMSSSSVTTKRSKADSKSCKMGGNWDTTDNRYGKYGTVTDKMKDATGRSRGGSKSSVDVVKTHDGKVDKRARDDKTGKVGGGDVRKSWGTDAMDKDTGSRGGVTYDSADAVDRVCNKDKDRKKRARHMKSSNNGGNNGGNNMNRRGGNGNGDNMYNMMGGYNMMNAMTDYYKMYYMKTGMDYTMYMMAMMMGAMNAMTNDSNATGSASDSDNNKSNDVTGNTSNTDSGSNNGKGSYNDDHNSGYGYNRDRGDRDRNDRDRDYNHRSGGNHRRNGRGGRGGYNRRNNGYHYNR

SEQ ID NO: 26 - protein, 256 aa, Saccharomyces cerevisiae
MSATHVSVVDAVHADAVSASAANDVSNAYGSHSVDYAHHHYYGHMHGRMHHRGSNTRVRDVSNGGMKVKNGAVASAAKAVHGKSANVVYSKAKRYRTMKNGCSWDKDARNSTTSSVNTRDDGTGASVARNNRGSVTVRDDNRRSNRGGRGRGGRGGRGGRGGSRGGGGRGGGGRGGYGGYSRGGYGGYSRGGYGGSRGGYDSRGGYDSRGGYSRGGYGGRNDYGRGSYGGSRGGYDGRGDYGRDAYRTRDARRSTR

SEQ ID NO: 27 - protein, 286 aa, Saccharomyces cerevisiae
MSDIEEGTPTNNGQQKERRKIEIKFIENKTRRHVTFSKRKHGIMKKAFELSVLTGTQVLLLVVSETGLVYTFSTPKFEPIVTQQEGRNLIQACLNAPDDEEEDEEEDGDDDDDDDDDGNDMQRQQPQQQQPQQQQQVLNAHANSLGHLNQDQVPAGALKQEVKSQLLGGANPNQNSMIQQQQHHTQNSQPQQQQQQQPQQQMSQQQMSQHPRPQQGIPHPQQSQPQQQQQQQQQLQQQQQQQQQQPLTGIHQPHQQAFANAASPYLNAEQNAAYQQYFQEPQQGQY

SEQ ID NO: 28 - protein, 414 aa, Saccharomyces cerevisiae
MAKTTKVKGNKKEVKASKQAKEEKAKAVSSSSSESSSSSSSSSESESESESESESSSSSSSSDSESSSSSSSDSESEAETKKEESKDSSSSSSDSSSDEEEEEEKEETKKEESKESSSSDSSSSSSSDSESEKEESNDKKRKSEDAEEEEDEESSNKKQKNEETEEPATIFVGRLSWSIDDEWLKKEFEHIGGVIGARVIYERGTDRSRGYGYVDFENKSYAEKAIQEMQGKEIDGRPINCDMSTSKPAGNNDRAKKFGDTPSEPSDTLFLGNLSFNADRDAIFELFAKHGEVVSVRIPTHPETEQPKGFGYVQFSNMEDAKKALDALQGEYIDNRPVRLDFSSPRPNNDGGRGGSRGFGGRGGGRGGNRGFGGRGGARGGRGGFRPSGSGANTAPLGRSRNTASFAGSKKTFD

SEQ ID NO: 29 - protein, 405 aa, Saccharomyces cerevisiae
MDTDKLISEAESHFSQGNHAEAVAKLTSAAQSNPNDEQMSTIESLIQKIAGYVMDNRSGGSDASQDRAAGGGSSFMNTLMADSKGSSQTQLGKLALLATVMTHSSNKGSSNRGFDVGTVMSMLSGSGGGSQSMGASGLAALASQFFKSGNNSQGQGQGQGQGQGQGQGQGQGSFTALASLASSFMNSNNNNQQGQNQSSGGSSFGALASMASSFMHSNNNQNSNNSQQGYNQSYQNGNQNSQGYNNQQYQGGNGGYQQQQGQSGGAFSSLASMAQSYLGGGQTQSNQQQYNQQGQNNQQQYQQQGQNYQHQQQGQQQQQGHSSSFSALASMASSYLGNNSNSNSSYGGQQQANEYGRPQHNGQQQSNEYGRPQYGGNQNSNGQHESFNFSGNFSQQNNNGNQNRY

SEQ ID NO: 30 - protein, 964 aa, Saccharomyces cerevisiae
MPEQAQQGEQSVKRRRVTRACDECRKKKVKCDGQQPCIHCTVYSYECTYKKPTKRTQNSGNSGVLTLGNVTTGPSSSTVVAAAASNPNKLLSNIKTERAILPGASTIPASNNPSKPRKYKTKSTRLQSKIDRYKQIFDEVFPQLPDIDNLDIPVFLQIFHNFKRDSQSFLDDTVKEYTLIVNDSSSPIQPVLSSNSKNSTPDEFLPNMKSDSNSASSNREQDSVDTYSNIPVGREIKIILPPKAIALQFVKSTWEHCCVLLRFYHRPSFIRQLDELYETDPNNYTSKQMQFLPLCYAAIAVGALFSKSIVSNDSSREKFLQDEGYKYFIAARKLIDITNARDLNSIQAILMLIIFLQCSARLSTCYTYIGVAMRSALRAGFHRKLSPNSGFSPIEIEMRKRLFYTIYKLDVYINAMLGLPRSISPDDFDQTLPLDLSDENITEVAYLPENQHSVLSSTGISNEHTKLFLILNEIISELYPIKKTSNIISHETVTSLELKLRNWLDSLPKELIPNAENIDPEYERANRLLHLSFLHVQIILYRPFIHYLSRNMNAENVDPLCYRRARNSIAVARTVIKLAKEMVSNNLLTGSYWYACYTIFYSVAGLLFYIHEAQLPDKDSAREYYDILKDAETGRSVLIQLKDSSMAASRTYNLLNQIFEKLNSKTIQLTALHSSPSNESAFLVTNNSSALKPHLGDSLQPPVFFSSQDTKNSFSLAKSEESTNDYAMANYLNNTPISENPLNEAQQQDQVSQGTTNMSNERDPNNFLSIDIRLDNNGQSNILDATDDVFIRNDGDIPTNSAFDFSSSKSNASNNSNPDTINNNYNNVSGKNNNNNNITNNSNNNHNNNNNDNNNNNNNNNNNNNNNNNSGNSSNNNNNNNNNKNNNDFGIKIDNNSPSYEGFPQLQIPLSQDNLNIEDKEEMSPNIEIKNEQNMTDSNDILGVFDQLDAQLFGKYLPLNYPSE

SEQ ID NO: 31 - protein, 758 aa, Saccharomyces cerevisiae
MDNTTNINTNERSSNTDFSSAPNIKGLNSHTQLQFDADSRVFVSDVMAKNSKQLLYAHIYNYLIKNNYWNSAAKFLSEADLPLSRINGSASGGKTSLNASLKQGLMDIASKGDIVSEDGLLPSKMLMDANDTFLLEWWEIFQSLFNGDLESGYQQDHNPLRERIIPILPANSKSNMPSHFSNLPPNVIPPTQNSFPVSEESFRPNGDGSNFNLNDPTNRNVSERFLSRTSGVYDKQNSANFAPDTAINSDIAGQQYATINLHKHFNDLQSPAQPQQSSQQQIQQPQHQPQHQPQQQQQQQQQQQQQQQQQQQQQQQQQQQQQQHQQQQQTPYPIVNPQMVPHIPSENSHSTGLMPSVPPTNQQFNAQTQSSMFSDQQRFFQYQLHHQNQGQAPSFQQSQSGRFDDMNAMKMFFQQQALQQNSLQQNLGNQNYQSNTRNNTAEETTPTNDNNANGNSLLQEHIRARFNKMKTIPQQMKNQSTVANPVVSDITSQQQYMHMMMQRMAANQQLQNSAFPPDTNRIAPANNTMPLQPGNMGSPVIENPGMRQTNPSGQNPMINMQPLYQNVSSAMHAFAPQQQFHLPQHYKTNTSVPQNDSTSVFPLPNNNNNNNNNNNNNNNNNSNNSNNNNNNNNNNNNSNNTPTVSQPSSKCTSSSSTTPNITTTIQPKRKQRVGKTKTKESRKVAAAQKVMKSKKLEQNGDSAATNFINVTPKDSGGKGTVKVQNSNSQQQLNGSFSMDTETFDIFNIGDFSPDLMDS

SEQ ID NO: 32 - protein, 750 aa, Saccharomyces cerevisiae
MTSVNRSNNTRSMSASRSATSRVRNTTANSSDVNSSKRNSNSVYDDNSSKRRSRRSDGKNNDHTYRTTVKSKNSRYVSSSKRAKRNSVGTSSASKSSNGGSAHKWSNMKNVSNSAVDAGSDSKSVGGRKSNNSNDKDNSARDDNNSGNNNNNNNHSSNNNDNNNNNNDDNNNNNNSNSRDNNNNSDDSNRNDSCKASNKRSGAKYKVVKRCSTNSTTKSWTYKNTDVNNYVTTTASHDVGVYRRRWVYGTTDVKNSNMDVCCTHVVSSTMSDSKYSTWRGDSRMAAYSSDWKSAHWYTAMKYYNHGKYYHMSTVNTAVNGKSVCTTSYMVDNYRAVRNNGNRNSYKHSAMSSDNVVSYKGDANGCNNADMVNDKYRHGSASHVGGKNAKYKRKDKKRKKSSNNDSSVTSSTGNSRNDNDDDMSSTTSSDHDANDDTRRSMTNAWTKNMTSKCGVRKHGGAHWYSCKSSSDVSKWMVKRAWDTMVTMNVVYDNTSNSGDCDDYDKSSNGGCWGTWDTCKNTHSSSDNGKDYMADSTDGDKDNGKWKRACRTRSRSGVRNDYRSSNTNGSVKCNHNNVGASDSARSNNTDHAVSVNGDNHYVGYKKRADYTCDKNGSASYTTWYVNSNNTNDNNYNSKNGCKSDYDKTTYVDATSWRHSARKANRRACTTRRKSKDNVMAATRGTRYYNKVRTGNVATHNTWRTHVDVSVMKAKSASRSRRNYVVSDDDAMKKKAKKTSTRVSCTKGRHCTD

SEQ ID NO: 33 - protein, 710 aa, Saccharomyces cerevisiae
MDNKRYNGNSNVDGTYDRNDTRMNTNARSVRVSDKRGRSSSTSKGSYRTRAGRSDTTNSSAKHHSKKSTVVVVTSSTDSNSTTYARVSSDSTVATSSTTTRTRTRNNTVSSTASSSTTDVGNATSANWSANASNTSSSDYATSYTRKSTDNYTTANSKNGNNWSSAGNSNTDHNTVNRRSSSTTNRVYTDAYYANYVVRVKSTSSVDDVDASNWTANKVVNSATNTSSNVTHNAVNTSTSATCSYGKVSARTRGNMAVSTVSACAAGKSKVGASTVSARVMYNVNGNNTKNHGVNYSTSNNTYCNTNSHSSNNYSSDSKKDHTSSKYDHNHNAKNKGVSDTNYGHNSKVKRKDTDAKRRKDSNSSTMAVMDSSDYGNTVKNSSNRDMRKCNKYTSMGVHKNGTWVCKKMANTRNVTSGVSDYCTNDGNYVGKGWNSSVSHWTVNRYGSRAVRACADSTCTTSVSYATDTNGTTWDTCTNKNCDKVNKNVKCCHKGSTVKNRGGASKNKHADGSSDSDGNYGTYKVTSRDNSVRDATKRNSNNSRVGSSAGSKSSKNHRKHGHSGRARGVSVSSVRSSNSRHNSVMNNAGTANNAMSNSYNNVVYSGNNNNGNSNGDNSDSRANGTNSVNNVSNNNNNYNNSGYSSMNSRSVSHNNNNNTNNYNNNDNDNNNNNNNNNNNNNNNNNNNNNSNNSNNNNNNDTSYRYRSYGY

SEQ ID NO: 34 - protein, 477 aa, Saccharomyces cerevisiae
DTKGYDDDAATDGKKHRRYRYVSGSVSGKRWTDGVSWSSRSGKYKDKNAGSNANATSSGSTDSAVTDGTSGARNNSSSKKKNHDTMGHSSSDTSSSNRSNKYTGVKKTSVKKRNSNHVSYYSVKDKNCVTKASKDVRSVAMGNTTGNVKNNSTTTGNGNNNNKSNSSTNTVSTNNNSANNAAGSNTSANKNYYYKNDSSGYTAASTTMYTANYTSDNTNATGMNTHVNNNNNNSNNSSNSNNSNNNNNNNNNNNNNNNNNNNNNVNTNAGNGNNNRHNASAYNTTGDNGSYYYTTNNNYYTTNVTNASTNNGYSTSSTHYYGHTSSASAAAGATGTGTANVVSSMHANNNSASSATSTAYVYSMNVNVYYNSSASAYKRANTTSNTNASGATSTNSGTMSNAYANSYTSVYYGYAMASANSMYHHHTVYATNMSSGHTSTGSDHHHYNDHKNAMGHANNNNTNNDTMNNNTNTSTTT

SEQ ID NO: 35 - protein, 454 aa, Saccharomyces cerevisiae
MDVRAACSASGRTGKKGYSYKMSNSGGSSSGGSDVGSTNGSNRAKNTNYKKTNKKYKATDKANDTKYYSNDKKSKRSANSMNDKDKCRTTNKDMTRYDSKSKVTNCDHKASSHSMKYKKRSVDKDHVMKDDSSVKASKMNSHNYSTNTMNKMDVYTKANMANKKKSDTSTWKNKNKSHVSYNNDKSKTKWYNDSDDDDDNNVNNNDNNNNNKNDNNNDNNNDTSNNNNNNNNRTKNNRNNRDWKTKKCTDMNDKRDNNNKNDMARNDNKNYNNVNKRNHKSSCRRDGYSANNAVNSTHASNKNVNDMNNDTYKNKTDTNKKNDSNSNDVTRKKRKTSDGNYSRNNVSVSRSKATTKKTKKKKRRDGKDKKNKKNADNKKNNAVTVSVYDSNKVKSNKRSRKVNNKSDVVNSGKDSRVKSCKKYADNNTKSNDADGWDDMNWVDRGCATTRWRAK

SEQ ID NO: 36 - protein, 284 aa, Saccharomyces cerevisiae
MNVTSKDGNHSSKKNRNTNKRHKNASNDRDSVSSNTTSMTDDADYNGASRTKNNSDSDRSNDTKNNYNKRTGYNYNGSGNRYTRKRTANKAYSDDNVKDDNNTKKASRSSGRNVNTRNKSKSHKVKNNKSSSRKSSAARKGKYNSNSDSTTRKVTDVKKRSKWHRHDKKMVKKSRYRKRMRGTDVSSSDNSKSTTKSYVSKNSAMNNNDVTDNKKTNNNKARDSMHTKKDTKDDTDSKKRKVVTNDNAAMVNKGWRKNVMMYKKSGNMKKYRYWTCYCNYVYYR

SEQ ID NO: 37 - DNA, 29 bp, Artificial Sequence
  Description of Artificial Sequence primer
gggaattcccattaccgacatttgggcgc

SEQ ID NO: 38 - DNA, 29 bp, Artificial Sequence
  Description of Artificial Sequence primer
ggggattctgattgattgattgattgtac

SEQ ID NO: 39 - DNA, 720 bp, Artificial Sequence
  Description of Artificial Sequence superbright GFP encoding sequence
atggctagcaaaggagaagaactcttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcacttatggtgttcagtgcttttcaagatacccggatcatatgaaacggcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttgggcacaaattggaatacaactataactcacacaatgtatacatcatggcagacaaacaaaagaatggaatcaaagctaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaatga

SEQ ID NO: 40 - protein, 239 aa, Artificial Sequence
  Description of Artificial Sequence superbright GFP encoding sequence
MASKGEELFTGVVPILVELDGDVNGHKFSVSGEGEGDATYGKLTLKFICTTGKLPVPWPTLVTTFTYGVQCFSRYPDHMKRHDFFKSAMPEGYVQERTIFFKDDGNYKTRAEVKFEGDTLVNRIELKGIDFKEDGNILGHKLEYNYNSHNVYIMADKQKNGIKANFKIRHNIEDGSVQLADHYQQNTPIGDGPVLLPDNHYLSTQSALSKDPNEKRDHMVLLEFVTAAGITHGMDELYK

SEQ ID NO: 41 - DNA, 27 bp, Artificial Sequence
  Description of Artificial Sequence synthetic primer
gaccgcggatggctagcaaaggagaag

SEQ ID NO: 42 - DNA, 28 bp, Artificial Sequence
  Description of Artificial Sequence synthetic primer
cctgagctctcatttgtatagttcatcc

SEQ ID NO: 43 - DNA, 34 bp, Artificial Sequence
  Description of Artificial Sequence synthetic primer
ggaggatccatggatacggataagttaatctcag

SEQ ID NO: 44 - DNA, 36 bp, Artificial Sequence
  Description of Artificial Sequence synthetic primer
ggaccgcgggtagcggttctgttgagaaaagttgcc

SEQ ID NO: 45 - DNA, 7239 bp, Artificial Sequence
  Description of Artificial Sequence vector containing chimeric gene
gacgaaagggcctcgtgatacgcctatttttataggttaatgtcatgataataatggtttcttaggacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaaggacccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcgtatgttgtgtggaattgtgagcggataacaatttcacacaggaaacagctatgaccatgattacgccaagctcggaattaaccctcactaaagggaacaaaagctgggtaccgggccccccctcgaggtcgacggtatcgataagcttgatatcgaattcccattaccgacatttgggcgctatacgtgcatatgttcatgtatgtatctgtatttaaaacacttttgtattatttttcctcatatatgtgtataggtttatacggatgatttaattattacttcaccaccctttatttcaggctgatatcttagccttgttactagttagaaaaagacatttttgctgtcagtcactgtcaagagattcttttgctggcatttcttctagaagcaaaaagagcgatgcgtcttttccgctgaaccgttccagcaaaaaagactaccaacgcaatatggattgtcagaatcatataaaagagaagcaaataactccttgtcttgtatcaattgcattataatatcttcttgttagtgcaatatcatatagaagtcatcgaaatagatattaagaaaaacaaactgtacaatcaatcaatcaatcaggatccatggatacggataagttaatctcagaggctgagtctcatttttctcaaggaaaccatgcagaagctgttgcgaagttgacatccgcagctcagtcgaaccccaatgacgagcaaatgtcaactattgaatcattaattcaaaaaatcgcaggatacgtcatggacaaccgtagtggtggtagtgacgcctcgcaagatcgtgctgctggtggtggttcatcttttatgaacactttaatggcagactctaagggttcttcccaaacgcaactaggaaaactagctttgttagccacagtgatgacacactcatcaaataaaggttcttctaacagagggtttgacgtagggactgtcatgtcaatgctaagtggttctggcggcgggagccaaagtatgggtgcttccggcctggctgccttggcttctcaattctttaagtcaggtaacaattcccaaggtcagggacaaggtcaaggtcaaggtcaaggtcaaggacaaggtcaaggtcaaggttcttttactgctttggcgtctttggcttcatctttcatgaattccaacaacaataatcagcaaggtcaaaatcaaagctccggtggttcctcctttggagcactagcttctatggcaagttcttttatgcattccaataataatcagaactccaacaatagtcaacagggttataaccaatcctatcaaaacggtaaccaaaatagtcaaggttacaataatcaacagtaccaaggtggcaacggtggttaccaacaacaacagggacaatctggtggtgctttttcctcattggcctccatggctcaatcttacttaggtggtggacaaactcaatccaaccaacagcaatacaatcaacaaggccaaaacaaccagcagcaataccagcaacaaggccaaaactatcagcaccaacaacagggtcagcagcagcaacaaggccactccagttcattctcagctttggcttccatggcaagttcctacctgggcaataactccaattcaaattcgagttatgggggccagcaacaggctaatgagtatggtagaccacaacacaatggtcaacaacaatctaatgagtacggaagaccgcaatacggcggaaaccagaactccaatggacagcacgaatcctttaatttttctggcaacttttctcaacagaacaataacggcaaccagaaccgctacccgcggatggctagcaaaggagaagaactcttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcacttatggtgttcagtgcttttcaagatacccggatcatatgaaacggcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttgggcacaaattggaatacaactataactcacacaatgtatacatcatggcagacaaacaaaagaatggaatcaaagctaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaatgagagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatagggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctgtggtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 46 - protein, 741 aa, Pichia pinus
MSQDQQQQQQFNANNLAGNVQNINLNAPAYDPAVQSYIPNTAQAFVPSAQPYIPGQQEQQFGQYGQQQQNYNQGGYNNYNNRGGYSNNRGGYNNSNRGGYSNYNSYNTNSNQGGYSNYNNNYANNSYNNNNNYNNNYNQGYNNYNSQPQGQDQQQETGSGQMSLEDYQKQQKESLNKLNTKPKKVLKLNLNSSTVKAPIVTKKKEEEPVNQESKTEEPAKEEIKNQEPAEAENKVEEESKVEAPTAAKPVSESEFPASTPKTEAKASKEVAAAAAALKKEVSQAKKESNVTNADALVKEQEEQIDASIVNDMFGGKDHMSIIFMGHVDAGKSTMGGNLLFLTGAVDKRTVEKYEREAKDAGRQGWYLSWIMDTNKEERNDGKTIEVGKSYFETDKRRYTILDAPGHKLYISEMIGGASQADVGVLVISSRKGEYEAGFERGGQSREHAILAKTQGVNKLVVVINKMDDPTVNWSKERYEECTTKLAMYLKGVGYQKGDVLFMPVSGYTGAGLKERVSQKDAPWYNGPSLLEYLDSMPLAVRKINDPFMLPISSKMKDLGTVIEGKIESGHVKKGQNLLVMPNKTQVEVTTIYNETEAEADSAFCGEQVRLRLRGIEEEDLSAGYVLSSINHPVKTVTRFEAQIAIVELKSILSTGFSCVMHVHTAIEEVTFTQLLHNLQKGTNRRSKKAPAFAKQGMKIIAVLETTEPVCIESYDDYPQLGRFTLRDQGQTIAIGKVTKLL

SEQ ID NO: 47 - protein, 715 aa, Candida albicans
MANASLNGDQSKQQQQQQQQQQQQQNYYNPNAAQSFVPQGGYQQFQQFQPQQQQQQYGGYNQYNQYQGGYQQNYNNRGGYQQGYNNRGGYQQNYNNRGGYQGYNQNQQYGGYQQYNSQPQQQQQQQSQGMSLADFQKQKTEQQASLNKPAVKKTLKLAGSSGIKLANATKKVDTTSKPQSKESSPAPAPAASASASAPQEEKKEEKEAAAATPAAAPETKKETSAPAETKKEATPTPAAKNESTPIPAAAAKKESTPVSNSASVATADALVKEQEDEIDEEVVKDMFGGKDHVSIIFMGHVDAGKSTMGGNILYLTGSVDKRTVEKYEREAKDAGRQGWYLSWVMDTNKEERNDGKTIEVGKAYFETDKRRYTILDAPGHKMYVSEMIGGASQADVGILVISARKGEYETGFEKGGQTREHALLAKTQGVNKIIVVVNKMDDSTVGWSKERYQECTTKLGAFLKGIGYAKDDIIYMPVSGYTGAGLKDRVDPKDCPWYDGPSLLEYLDNMDTMNRKINGPFMMPVSGKMKDLGTIVEGKIESGHVKKGTNLIMMPNKTPIEVLTIFNETEQECDTAFSGEQVRLKIKGIEEEDLQPGYVLTSPKNPVKTVTRFEAQIAIVELKSILSNGFSCVMHLHTAIEEVKFIELKHKLEKGTNRKSKKPPAFAKKGMKIIAILEVGELVCAETYKDYPQLGRFTLRDQGTTIAIGKITKLL

SEQ ID NO: 48 - DNA, 653 bp, Saccharomyces cerevisiae
tcgagtttatcattatcaatactcgccatttcaaagaatacgtaaataattaatagtagtgattttcctaactttatttagtcaaaaaattagccttttaattctgctgtaacccgtacatgccaaaatagggggcgggttacacagaatatataacactgatggtgcttgggtgaacaggtttattcctggcatccactaaatataatggagcccgctttttaagctggcatccagaaaaaaaaagaatcccagcaccaaaatattgttttcttcaccaaccatcagttcataggtccattctcttagcgcaactacagagaacagggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggtttaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcga

SEQ ID NO: 49 - DNA, 7988 bp, Artificial Sequence
  Description of Artificial Sequence Ure2N-Sup35C integration plasmid
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattggagctccaccgcggtgaaaagagtcagtgagacgacgacttcaggatctttgggtttcaggatatgtggcatgaaaatacagaaaaatccctctgtgctgaatcagctttcgctcgaatattacgaagaagaagcagacagtgattatatctttataaacaaattgtatggtcgttcaagaaccgatcaaaatgtttcagatgcaattgaactttattttaacaatcctcatctgtcggatgcgagaaagcatcaactgaagaaaacatttttgaaaagattgcagttgttttataatactatgctagaagaagaagttagaatgatatcaagtagtcttttgtttatttacgaaggagacccggagcgatgggaattactaaatgatgttgacaaacttatgcgagatgattttatagacgatgatgacgacgatgatgataatgatgatgatgatgatgatgatgccgagggaagcagcgaaggaccaaaggacaaaaaaacaactggttctttgagttccatgtcactaatagattttgcacattctgaaataacgccggggaagggttatgatgaaaacgtgattgaaggagttgaaaccttgctagatatttttatgaaattctagatatattgagaggtgaagtttaccttgtttatggtatatggtacaaaaagaactaaactaattatacgtctatatatatatatatatatataacagctttattaaaccttgttttttaatatagaagaaaatgctttatgatcggtattattgtgtttgcatttacttatgtttgcaagaaatggatccttactcggcaattttaacaattttaccaattgctattgtggtaccttgatctctcaaagtgaatctacctaattgagggtaatcttggtaagtttccacacaaactggagcttcagtttctaaaacagcgatgaccttcatacccttcttagcaaaagcaggtggtttctttgacttacggttggtacccttttctaatttgtgcaataacttaacaatatgtacctcttcaattgctgtatgaacatgcataacacatgaaaaaccggctgctatgatagattttaattctacaatagcaatttgagctacaaacttggtaacactcttgatagggttctttggcgatgttagtacaaaacctggtgaaatgtcttcttcttcaacacctttgattcttagtttaacttgctcaccacacatagccatatcaacttcattttcagtttcgttgtaaatattttgaatttccacagcggttttgttaggcatcagtagggtggattgaccctttttgatatgaccggattcaattttaccttcaacgatggtacctagatccttcatcttagcggcaataggcaacatgaatggagcattgatgtgacggtcgacgtggttcattgtatccagatattctaacagagttgggccggtgtaccatgggcattcttttggatctacgtgatctttcaaatttgcaccactgtagccggatactggcataaatacaacgtctgtcttaatgttgtaaccaattgctctcaagaaattgctgacattactcacacattggtcgtaacgttccttagaccagttaacggttgggtcatccatcttatttacgacgacaaccatcttattaacaccttgggtcttggccaatagggcgtgttcacgagtttgaccacctctctcaaaaccggtttcgtactcaccctttctggcggaaatgaccaaaacaccaacatcagcttgagaagcaccaccgatcatctcggaaacgtacattttatgaccaggagcatccaatatggtataacgccttttttcagtttcaaagtaggccttaccaacttcgatagtcttaccatcatttctttcttctttgttggtatccatgacccatgacaagtaccaaccttgtctgcctgcatccttggcttctctttcatatttctcaatagttctcttatccacagagccagtcaagtatagtagattaccacccatagtagatttaccggcatcaacatgacccatgaaaattaaagaaacgtgatctttaccaccaaacatagcgtagtctgggacgtcgtatgggtagcggccgctgttattgttttgaacattattgttattactactgctattgttattattattattatttacacctgttgaaaattcaaaatttatattactttgatcggtggttgtattactgttcctgtttcctatgtttacttgacggagcgcattggagagattcgacacttggttgccgttgttattcatcatgaattctgttgctagtgggcagatatagatgttattccgagcaagtcgatgaagaaaccgctttttgttacagtacaatggagtctttcaagagaagatgtaccaatatacactacactcttcagaagcaatgggagctttggtcgagtgaaaaaaaaattttctccataaagaaagatcatattatacgatgatgtaagatataatacccggttgtaatgtacatttaagagcaaggtaagaagtgacaataacttctgtatgatcttagcatgtacctcttttggtgggctgagaactaagattcatctttttgcggaagaattttgctatgaacttcacaactttatgaagtggtttaagagaattacaaaagaaatgacacagactcgaacactgtgacgcgtcgtcttagtaaaaaataataatttgagtcaaatagcgcagctaatgcgaaacaaagaaatgaagcatataccattcgttgtatgatttttgtgtggttgacagatattctgccgaaattttaacgcttattataaatataaatgtatgtatgtgtgtataaacagatacgatattcaattttctaccgtagggttgggattttcttcaaactccaattcttcgtcgggtatttcctcaatggcgatcctcttttttggcttcggcttttcagtgtcattgacaattttaggcaccttaatttgtagtagaccgttgttgtaagtagctttaatttcttcgtccttaatgcgtggcagcacggggaatttaacggttctctcaaacgcaccatattttagttccgtgatcttcaagaatttttcatcaatgcccactctgtcttcgatcttacccttgatgagcatctcatgagaagatggatggtaatcaatgtggaaagccctagagttagcacctggtaacgcaagaacaactacgtaagtgtcctcggtatcatagacattcacttctggtgaaaatggtaagtccattctcgtttcaggcttggatacttgtaacgggtcaggtattggggaaggcgcttgtaggtgagcgaacgatgaagattttttggctaatggtggtctcgacgattcctccagctgattcaaaggtttttctttgttggtttcgccagcttcctctttgggtgcttcagacttatccttcttatccttttcttctcccttttcgccctcctgttcggtatttgcttcaatttctggttcagtgccttcatatggtggaacacctattaacgcggttaataagtcgtttaaactgtttgcctgttggttggtcctattattcctggcagtattacaatggtaatatgatggatatcttctcgagggggggcccggtacccagcttttgttccctttagtgagggttaattccgagcttggcgtaatcatggtcatagctgtttcctgtgtgaaattgttatccgctcacaattccacacaacatacgagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 50 - protein, 405 aa, Saccharomyces cerevisiae; identical to SEQ ID NO: 29

SEQ ID NO: 51 - protein, 128 aa, Saccharomyces cerevisiae
MSANDYYGGTAGEKSQYSRPSNPPPSSAHQNKTQERGYPPQQQQQYYQQQQQHPGYYNQQGYNQQGYNQQGYNQQGYNQQGYNQQGYNQQGHQQPVYVQQQPPQRGNEGCLAACLAALCICCTMDMLF

SEQ ID NO: 52 - protein, 534 aa, Saccharomyces cerevisiae
MSSDEEDFNDIYGDDKPTTTEEVKKEEEQNKAGSGTSQLDQLAALQALSSSLNKLNNPNSNNSSSNNSNQDTSSSKQDGTANDKEGSNEDTKNEKKQESATSANANANASSAGPSGLPWEQLQQTMSQFQQPSSQSPPQQQVTQTKEERSKADLSKESCKMFIGGLNWDTTEDNLREYFGKYGTVTDLKIMKDPATGRSRGFGFLSFEKPSSVDEVVKTQHILDGKVIDPKRAIPRDEQDKTGKIFVGGIGPDVRPKEFEEFFSQWGTIIDAQLMLDKDTGQSRGFGFVTYDSADAVDRVCQNKFIDFKDRKIEIKRAEPRHMQQKSSNNGGNNGGNNMNRRGGNFGNQGDFNQMYQNPMMGGYNPMMNPQAMTDYYQKMQEYYQQMQKQTGMDYTQMYQQQMQQMAMMMPGFAMPPNAMTLNQPQQDSNATQGSPAPSDSDNNKSNDVQTIGNTSNTDSGSPPLNLPNGPKGPSQYNDDHNSGYGYNRDRGDRDRNDRDRDYNHRSGGNHRRNGRGGRGGYNRRNNGYHPYNR

SEQ ID NO: 53 - DNA, 34 bp, Artificial Sequence; identical to SEQ ID NO: 43
  Description of Artificial Sequence synthetic primer

SEQ ID NO: 54 - DNA, 36 bp, Artificial Sequence
  Description of Artificial Sequence synthetic primer
ccaagctttcagtagcggttctgttgagaaaagttg

SEQ ID NO: 55 - DNA, 20 bp, Artificial Sequence
  Description of Artificial Sequence synthetic primer
ggtgtcttggccaattgccc

SEQ ID NO: 56 - DNA, 39 bp, Artificial Sequence
  Description of Artificial Sequence synthetic primer
gtcgacctgcagcgtacgcatttcagatctttgctatac

SEQ ID NO: 57 - DNA, 40 bp, Artificial Sequence
  Description of Artificial Sequence synthetic primer
cgagctcgaattcatcgattgattcagttcgccttctatc

SEQ ID NO: 58 - DNA, 22 bp, Artificial Sequence
  Description of Artificial Sequence synthetic primer
ctgttttgaaagggtccacatg

SEQ ID NO: 59 - DNA, 34 bp, Artificial Sequence; identical to SEQ ID NO: 43
  Description of Artificial Sequence synthetic primer

SEQ ID NO: 60 - DNA, 36 bp, Artificial Sequence; identical to SEQ ID NO: 44
  Description of Artificial Sequence synthetic primer

SEQ ID NO: 61 - DNA, 36 bp, Artificial Sequence
  Description of Artificial Sequence synthetic primer
gaggatccatgcctgatgatgaggaagaagacgagg

SEQ ID NO: 62 - DNA, 26 bp, Artificial Sequence
  Description of Artificial Sequence synthetic primer
cggaattcctcgagaagatatccatc

SEQ ID NO: 63 - DNA, 24 bp, Artificial Sequence
  Description of Artificial Sequence synthetic primer
gggatcctgttgctagtgggcaga

SEQ ID NO: 64 - DNA, 34 bp, Artificial Sequence
  Description of Artificial Sequence synthetic primer
gtaccgcggatgtctttgaacgactttcaaaagc

SEQ ID NO: 65 - DNA, 35 bp, Artificial Sequence
  Description of Artificial Sequence synthetic primer
gtggagctcttactcggcaattttaacaattttac